SEQUENCE LISTING (70 sequences)

SEQ ID NO: 1 - protein, 42 aa, Homo sapiens [sequence omitted]

SEQ ID NO: 2 - protein, 175 aa, Homo sapiens
AEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPK

SEQ ID NO: 3 - protein, 171 aa, Homo sapiens
AEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVR

SEQ ID NO: 4 - protein, 165 aa, Homo sapiens
AEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPK

SEQ ID NO: 5 - protein, 150 aa, Homo sapiens
AEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSR

SEQ ID NO: 6 - protein, 121 aa, Homo sapiens
AEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPK

SEQ ID NO: 7 - protein, 67 aa, Homo sapiens
AEPRQEFEVMEDHAGTYGLGDRKDQGGYTMHQDQEGDTDAGLKAEEAGIGDTPSLEDEAAGHVTQAR

SEQ ID NO: 8 - protein, 180 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSS

SEQ ID NO: 9 - protein, 23 aa, Homo sapiens
AEPRQEFEVMEDHAGTYGLGDRK

SEQ ID NO: 10 - protein, 23 aa, Macaca fascicularis
AEPRQEFDVMEDHAGTYGLGDRK

SEQ ID NO: 11 - protein, 23 aa, Homo sapiens
AEPRQEFEVMEDHAGTYGLGDRK

SEQ ID NO: 12 - protein, 23 aa, Macaca fascicularis
AEPRQEFDVMEDHAGTYGLGDRK

SEQ ID NO: 13 - protein, 6 aa, Homo sapiens
DQGGYT

SEQ ID NO: 14 - protein, 8 aa, Homo sapiens
GYTMHQDQ

SEQ ID NO: 15 - protein, 11 aa, Homo sapiens
VGSNKGAIIGL

SEQ ID NO: 16 - protein, 8 aa, Homo sapiens
KGAIIGLM

SEQ ID NO: 17 - protein, 17 aa, Homo sapiens
DENPVVHFFKNIVTPRT

SEQ ID NO: 18 - protein, 21 aa, Homo sapiens
KSHGRTQDENPVVHFFKNIVT

SEQ ID NO: 19 - protein, 15 aa, Homo sapiens
ARTAHYGSLPQKSHG

SEQ ID NO: 20 - protein, 15 aa, Homo sapiens
HHPARTAHYGSLPQK

SEQ ID NO: 21 - protein, 15 aa, Homo sapiens
YGSLPQKSHGRTQDE

SEQ ID NO: 22 - protein, 18 aa, Homo sapiens
TQDENPVVHFFKNIVTPR

SEQ ID NO: 23 - protein, 16 aa, Homo sapiens
KNIVTPRTPPPSQGKG

SEQ ID NO: 24 - protein, 10 aa, Homo sapiens
NPVVHFFKNI

SEQ ID NO: 25 - protein, 10 aa, Homo sapiens
PVVHFFKNIV

SEQ ID NO: 26 - protein, 10 aa, Homo sapiens
VVHFFKNIVT

SEQ ID NO: 27 - protein, 10 aa, Homo sapiens
VHFFKNIVTP

SEQ ID NO: 28 - protein, 52 aa, Artificial Sequence
  Description of Artificial Sequence Synthetic polypeptide
EAYKAAEKAYAAKEAAKEAAKAKAEKKAAYAKAKAAKYEKKAKKAAAEYKKK

SEQ ID NO: 29 - protein, 21 aa, Homo sapiens
MEVGWYRPPFSRVVHLYRNGK

SEQ ID NO: 30 - protein, 21 aa, Homo sapiens
MEVGWYRSPFSRVVHLYRNGK

SEQ ID NO: 31 - protein, 20 aa, Homo sapiens
GQFRVIGPRHPIRALVGDEV

SEQ ID NO: 32 - protein, 23 aa, Homo sapiens
GGFTCFFRDHSYQEEAAMELKVE

SEQ ID NO: 33 - protein, 16 aa, Homo sapiens
VFLCLQYRLRGKLRAE

SEQ ID NO: 34 - protein, 15 aa, Homo sapiens
LVALIICYNWLHRRL

SEQ ID NO: 35 - protein, 15 aa, Homo sapiens
ENPVVHFFKNIVTPR

SEQ ID NO: 36 - protein, 20 aa, Homo sapiens
VDAQGTLSKIFKLGGRDSRS

SEQ ID NO: 37 - protein, 16 aa, Homo sapiens
CHCLGKWLGHPDKFVG

SEQ ID NO: 38 - protein, 22 aa, Homo sapiens
GAVRQIFGDYKTTICGKGLSAT

SEQ ID NO: 39 - protein, 15 aa, Artificial Sequence
  Description of Artificial Sequence Synthetic peptide
AAAYAAAAAAKAAAA

SEQ ID NO: 40 - protein, 15 aa, Artificial Sequence
  Description of Artificial Sequence Synthetic peptide
AEKYAAAAAAKAAAA

SEQ ID NO: 41 - protein, 15 aa, Artificial Sequence
  Description of Artificial Sequence Synthetic peptide
AKEYAAAAAAKAAAA

SEQ ID NO: 42 - protein, 15 aa, Artificial Sequence
  Description of Artificial Sequence Synthetic peptide
AKKYAAAAAAKAAAA

SEQ ID NO: 43 - protein, 15 aa, Artificial Sequence
  Description of Artificial Sequence Synthetic peptide
AEAYAAAAAAKAAAA

SEQ ID NO: 44 - protein, 15 aa, Artificial Sequence
  Description of Artificial Sequence Synthetic peptide
KEAYAAAAAAKAAAA

SEQ ID NO: 45 - protein, 15 aa, Artificial Sequence
  Description of Artificial Sequence Synthetic peptide
AEEYAAAAAAKAAAA

SEQ ID NO: 46 - protein, 15 aa, Artificial Sequence
  Description of Artificial Sequence Synthetic peptide
AAEYAAAAAAKAAAA

SEQ ID NO: 47 - protein, 15 aa, Artificial Sequence
  Description of Artificial Sequence Synthetic peptide
EKAYAAAAAAKAAAA

SEQ ID NO: 48 - protein, 15 aa, Artificial Sequence
  Description of Artificial Sequence Synthetic peptide
AAKYEAAAAAKAAAA

SEQ ID NO: 49 - protein, 15 aa, Artificial Sequence
  Description of Artificial Sequence Synthetic peptide
AAKYAEAAAAKAAAA

SEQ ID NO: 50 - protein, 15 aa, Artificial Sequence
  Description of Artificial Sequence Synthetic peptide
EAAYAAAAAAKAAAA

SEQ ID NO: 51 - protein, 15 aa, Artificial Sequence
  Description of Artificial Sequence Synthetic peptide
EKKYAAAAAAKAAAA

SEQ ID NO: 52 - protein, 15 aa, Artificial Sequence
  Description of Artificial Sequence Synthetic peptide
EAKYAAAAAAKAAAA

SEQ ID NO: 53 - protein, 15 aa, Artificial Sequence
  Description of Artificial Sequence Synthetic peptide
AEKYAAAAAAAAAAA

SEQ ID NO: 54 - protein, 15 aa, Artificial Sequence
  Description of Artificial Sequence Synthetic peptide
AKEYAAAAAAAAAAA

SEQ ID NO: 55 - protein, 15 aa, Artificial Sequence
  Description of Artificial Sequence Synthetic peptide
AKKYEAAAAAAAAAA

SEQ ID NO: 56 - protein, 15 aa, Artificial Sequence
  Description of Artificial Sequence Synthetic peptide
AKKYAEAAAAAAAAA

SEQ ID NO: 57 - protein, 15 aa, Artificial Sequence
  Description of Artificial Sequence Synthetic peptide
AEAYKAAAAAAAAAA

SEQ ID NO: 58 - protein, 15 aa, Artificial Sequence
  Description of Artificial Sequence Synthetic peptide
KEAYAAAAAAAAAAA

SEQ ID NO: 59 - protein, 15 aa, Artificial Sequence
  Description of Artificial Sequence Synthetic peptide
AEEYKAAAAAAAAAA

SEQ ID NO: 60 - protein, 15 aa, Artificial Sequence
  Description of Artificial Sequence Synthetic peptide
AAEYKAAAAAAAAAA

SEQ ID NO: 61 - protein, 15 aa, Artificial Sequence
  Description of Artificial Sequence Synthetic peptide
EKAYAAAAAAAAAAA

SEQ ID NO: 62 - protein, 15 aa, Artificial Sequence
  Description of Artificial Sequence Synthetic peptide
AAKYEAAAAAAAAAA

SEQ ID NO: 63 - protein, 15 aa, Artificial Sequence
  Description of Artificial Sequence Synthetic peptide
AAKYAEAAAAAAAAA

SEQ ID NO: 64 - protein, 15 aa, Artificial Sequence
  Description of Artificial Sequence Synthetic peptide
EKKYAAAAAAAAAAA

SEQ ID NO: 65 - protein, 15 aa, Artificial Sequence
  Description of Artificial Sequence Synthetic peptide
EAKYAAAAAAAAAAA

SEQ ID NO: 66 - protein, 15 aa, Artificial Sequence
  Description of Artificial Sequence Synthetic peptide
AEYAKAAAAAAAAAA

SEQ ID NO: 67 - protein, 15 aa, Artificial Sequence
  Description of Artificial Sequence Synthetic peptide
AEKAYAAAAAAAAAA

SEQ ID NO: 68 - protein, 15 aa, Artificial Sequence
  Description of Artificial Sequence Synthetic peptide
EKYAAAAAAAAAAAA

SEQ ID NO: 69 - protein, 15 aa, Artificial Sequence
  Description of Artificial Sequence Synthetic peptide
AYKAEAAAAAAAAAA

SEQ ID NO: 70 - protein, 15 aa, Artificial Sequence
  Description of Artificial Sequence Synthetic peptide
AKYAEAAAAAAAAAA

(19) United States
(12) Patent Application Publication
(10) Pub. No.: US 2020/0330592 A1
(43) Pub. Date: Oct. 22, 2020

(54) COMPOSITIONS AND METHODS OF TREATING ALZHEIMER'S AND OTHER AMYLOID RELATED DISEASES

(71) Applicant: Keith BLACK, Los Angeles, CA (US)

(72) Inventor: Keith BLACK, Los Angeles, CA (US)

(21) Appl. No.: 16/754,106

(22) PCT Filed: Oct. 8, 2018
(86) PCT No.: PCT/US18/54854

§ 371 Date: Apr. 6, 2020

Related U.S. Application Data
(60) Provisional application No. 62/569,977, filed on Oct. 9, 2017.

(51) Int. Cl. A61K 39/395; A61K 38/17; A61P 25/28

(52) U.S. Cl. CPC A61K 39/3955; A61P 25/28; A61K 38/1709

(57) ABSTRACT

The present disclosure relates to methods of treating, delaying the onset, alleviating a symptom, and/or deterring the progression of a disease related to beta amyloid deposits, neurological damage, tauopathy, and/or neurodegeneration by administering a myelin sheath protein and an antibody, such as an anti-tau antibody and/or an antibody that binds amyloid beta. The present disclosure relates to methods of using a myelin sheath protein and/or an antibody, such as an anti-tau antibody and/or an antibody that binds amyloid beta, to treat or to slow the progression of a disease characterized in part by beta amyloid (A) expression or activity, or by aberrant deposition of beta amyloid in a subject, such as in Alzheimers disease, and the pathologies associated with such a disease, including for example, behavioral changes or cognitive dysfunction associated with Alzheimers disease.

### CROSS-REFERENCE

This application claims the priority benefit of U.S. provisional application 62/569,977 filed Oct. 9, 2017, which is hereby incorporated herein in its entirety for all purposes.

### SEQUENCE LISTING

The instant application contains a Sequence Listing which has been submitted electronically in ASCII format and is hereby incorporated by reference in its entirety. Said ASCII copy, created on Oct. 4, 2018, is named 50014-703_601_SL.txt and is 25,267 bytes in size.

### BACKGROUND

Alzheimer's disease (AD) can be an age-related progressive neurodegenerative disorder characterized by memory loss and severe cognitive decline (Hardy & Selkoe, Science. 2002 Jul. 19; 297(5580): 353-6). The clinical features are manifested morphologically by excessive accumulation of extracellular aggregations of amyloid beta-peptide (Abeta) in the form of amyloid plaques in the brain parenchyma, particularly in the hippocampus and cerebral cortex, leading to neuronal loss (Selkoe, Neuron. 1991 April; 6(4): 487-98). As of 2016, the number of diagnoses is reported to be 44 million-plus worldwide. AD is the 6^{th} leading cause of death in the United States, where an estimated 1 in 9 people over the age of 65 is living with AD. Thus, there is a need for therapeutically acceptable modalities for the treatment and/or prevention of AD, related neurodegenerative disorders, and amyloid related diseases associated with protein aggregation.

Citation of the above documents is not intended as an admission that any of the foregoing is pertinent prior art. All statements as to the date or representation as to the contents of these documents are based on the information available to the applicant and do not constitute any admission as to the correctness of the dates or contents of these documents.

### SUMMARY

Disclosed herein are methods of treating Alzheimer's disease, comprising administering to a subject in need thereof a pharmaceutical composition comprising an effective amount of an antibody or antigen-binding fragment thereof that binds to soluble beta amyloid (Abeta) in combination with a myelin sheath protein or a synthetic polypeptide comprising a structure similar to the protein, or a homolog thereof, or a fragment thereof, wherein the antibody is administered peripherally to the subject to exert its beneficial effects. In some embodiments, the myelin sheath protein or the synthetic polypeptide comprising a structure similar to the protein, or a homolog thereof, or a fragment thereof, comprises, consists essentially of, or consists of an amino acid sequence selected from the group consisting of: Asp-Glu-Asn-Pro-Val-Val-His-Phe-Phe-Lys-Asn-Ile-Val-Thr-Pro-Arg-Thr (SEQ ID NO:17); Lys-Ser-His-Gly-Arg-Thr-Gln-Asp-Glu-Asn-Pro-Val-Val-His-Phe-Phe-Lys-Asn-Ile-Val-Thr (SEQ ID NO: 18); Ala-Arg-Thr-Ala-His-Tyr-Gly-Ser-Leu-Pro-Gln-Lys-Ser-His-Gly (SEQ ID NO: 19); His-His-Pro-Ala-Arg-Thr-Ala-His-Tyr-Gly-Ser-Leu-Pro-Gln-Lys (SEQ ID NO: 20); Tyr-Gly-Ser-Leu-Pro-Gln-Lys-Ser-His-Gly-Arg-Thr-Gln-Asp-Glu (SEQ ID NO: 21); Thr-Gln-Asp-Glu-Asn-Pro-Val-Val-His-Phe-Phe-Lys-Asn-Ile-Val-Thr-Pro-Arg (SEQ ID NO: 22); Lys-Asn-Ile-Val-Thr-Pro-Arg-Thr-Pro-Pro-Pro-Ser-Gln-Gly-Lys-Gly (SEQ ID NO: 23); Asn-Pro-Val-Val-His-Phe-Phe-Lys-Asn-Ile (SEQ ID NO: 24); Pro-Val-Val-His-Phe-Phe-Lys-Asn-Ile-Val (SEQ ID NO: 25); Val-Val-His-Phe-Phe-Lys-Asn-Ile-Val-Thr (SEQ ID NO: 26); Val-His-Phe-Phe-Lys-Asn-Ile-Val-Thr-Pro (SEQ ID NO: 27); and Glu-Ala-Tyr-Lys-Ala-Ala-Glu-Lys-Ala-Tyr-Ala-Ala-Lys-Glu-Ala-Ala-Lys-Glu-Ala-Ala-Lys-Ala-Lys-Ala-Glu-Lys-Lys-Ala-Ala-Tyr-Ala-Lys-Ala-Lys-Ala-Ala-Lys-Tyr-Glu-Lys-Lys-Ala-Lys-Lys-Ala-Ala-Ala-Glu-Tyr-Lys-Lys-Lys (SEQ ID NO: 28). In some embodiments, the antibody comprises an immunoglobulin domain. In some embodiments, the antibody is a monoclonal antibody or a binding fragment thereof. In some embodiments, the antibody is a chimeric antibody or a fragment thereof. In some embodiments, the monoclonal antibody or binding fragment thereof is a human antibody, a humanized antibody, or a mouse antibody. In some embodiments, the monoclonal antibody or binding fragment thereof inhibits formation of amyloid deposits in the subject. In some embodiments, the monoclonal antibody or binding fragment thereof is an IgG class antibody. In some embodiments, the IgG class antibody is selected from the group consisting of IgG1, IgG2, IgG3, IgG4, and IgGM. In some embodiments, the antibody or antigen-binding fragment thereof that binds to soluble beta amyloid (Abeta)is selected from the group comprising LY2062430, RN-1219, R-1450, humanized m266, or any combination thereof. In some embodiments, the antibody or antigen-binding fragment thereof that binds to soluble beta amyloid (Abeta) binds Abeta 42. In some embodiments, the antibody or antigen-binding fragment thereof that binds to soluble beta amyloid (Abeta) binds Abeta 40. In some embodiments, the antibody or antigen-binding fragment thereof that binds to soluble beta amyloid (Abeta) binds an intracellular Abeta. In some embodiments, the antibody or antigen-binding fragment thereof is a bispecific antibody or an antigen-binding fragment having two antigen-binding regions. In some embodiments, one of the antigen-binding regions of the bispecific antibody or of the antigen-binding fragment having two antigen-binding regions binds to human tau or a fragment thereof. In some embodiments, one of the antigen-binding regions of the bispecific antibody or of the antigen-binding fragment having two antigen-binding regions binds to human amyloid protein or a fragment thereof. In some embodiments, one of the first antigen-binding region of the bispecific antibody or of the antigen-binding fragment having two antigen-binding regions binds to human beta amyloid, or amyloid-precursor-protein, or amyloid beta-derived diffusible ligands, or a fragment thereof, and the other antigen-binding region of the bispecific antibody or of the antigen-binding fragment having two antigen-binding regions binds to human tau or a fragment thereof. In some embodiments, the bispecific antibody or the antigen-binding fragment having two antigen-binding regions thereof comprises a first specificity towards: a) an epitope of beta-amyloid peptide that contains residues 28-35 of beta-amyloid; b) an epitope of beta-amyloid peptide that contains residues 28-34 of beta-amyloid; c) an epitope of beta-amyloid peptide that contains residues 28-33 of beta-amyloid; or d) an epitope within the region of residues 28-35 of beta-amyloid; and a second specificity towards a tau protein or a fragment thereof. In some embodiments, the administering of myelin sheath protein or a synthetic polypeptide comprising a structure similar to the protein, or a homolog thereof, or a fragment thereof, and the antibody or the antigen-binding fragment thereof results in a synergistic effect. In some embodiments, the synthetic polypeptide comprises an amino acid sequence at least about 80%, at least about 85%, at least about 90%, at least about 95%, at least about 97%, at least about 98%, or at least about 99% identical to a myelin sheath protein or a fragment thereof. In some embodiments, the method further comprises administering an antibody or antigen-binding fragment thereof that binds tau or a fragment thereof. In some embodiments, the antibody or antigen-binding fragment thereof that specifically binds tau is selected from the group comprising BMS-986168, C2N-8E12, AADVAC-1, AADVAC-2, ACI-35, RG7345, TRx-237-015 (LMTX), AV-1451, AV-680, Posiphen, or any combination thereof. In some embodiments, the subject has a genomic mutation in the APP gene. In some embodiments, the subject has a genomic mutation in the ApoE gene. In some embodiments, the subject has a genomic mutation in a presenilin gene.

In one embodiment, the present disclosure provides a method of treating, or delaying the onset, or the progression of a disease characterized at least in part by beta amyloid expression, activity, or deposition in a subject, or for ameliorating at least one symptom associated with the disease, the method comprising administering a therapeutically effective amount of a first agent and a second agent to the subject, wherein the first agent is a myelin sheath protein and/or a synthetic polypeptide comprising a structure similar to said protein, or a homolog thereof, or a fragment thereof, and the second agent is an antibody or an antigen-binding fragment thereof that specifically binds beta amyloid, and/or amyloid-precursor-protein, and/or amyloid beta-derived diffusible ligands, and/or tau, and/or a fragment thereof, or any combination thereof, wherein the first agent and the second agent when administered to the subject act synergistically to reduce, inhibit, or ameliorate the at least one symptom or pathogenesis of the disease.

In one embodiment, the present disclosure provides a method of improving cognitive impairment in a subject having beta amyloid deposits in brain tissue, the method comprising administering to the subject a therapeutically effective amount of at least one myelin sheath protein and/or a synthetic polypeptide comprising a structure similar to said protein, or a homolog thereof, or a fragment thereof, and an antibody or an antigen-binding fragment thereof that specifically binds beta amyloid, and/or amyloid-precursor-protein, and/or amyloid beta-derived diffusible ligand, and/or tau, and/or a fragment thereof, or any combination thereof, wherein the subject demonstrates an improvement in cognitive function without necessarily exhibiting a concurrent change in the beta amyloid plaque burden in the brain.

In one embodiment, the present disclosure provides a method of treating or preventing a neurological disorder, including delaying the onset, slowing the progression or ameliorating at least one symptom of the disorder, comprising administering a therapeutically effective amount of at least one myelin sheath protein and/or a synthetic polypeptide comprising a structure similar to said protein, or a homolog thereof, or a fragment thereof, before, after, or concurrently with at least one antibody or antigen-binding fragment thereof that specifically binds beta amyloid, and/or amyloid-precursor-protein, and/or amyloid beta-derived diffusible ligand, and/or tau, and/or a fragment thereof, and/or any combination thereof, to a subject in need thereof.

In one embodiment, the present disclosure provides a method of treating a subject suffering from an amyloid disease and/or tauopathy comprising administering to the subject a therapeutically effective amount of at least one myelin sheath protein and/or a synthetic polypeptide comprising a structure similar to said protein, or a homolog thereof, or a fragment thereof, and at least one antibody or antigen-binding fragment thereof that specifically binds tau, or a fragment thereof and/or at least one antibody or antigen-binding fragment thereof that specifically binds beta amyloid, and/or amyloid-precursor-protein, and/or amyloid beta-derived diffusible ligand, and/or a fragment thereof, and/or any combination thereof.

In one embodiment, the present disclosure provides a method of treating a subject having a brain Aβ disorder or predisposition to a brain Aβ disorder, comprising administering to the subject a therapeutically effective amount of at least one myelin sheath protein and/or a synthetic polypeptide comprising a structure similar to said protein, and/or a homolog thereof, and/or a fragment thereof, and at least one antibody or antigen-binding fragment thereof that specifically binds tau, and/or a fragment thereof, and/or at least one antibody or antigen-binding fragment thereof that specifically binds beta amyloid, and/or amyloid-precursor-protein, and/or amyloid beta-derived diffusible ligand, and/or a fragment thereof, and/or any combination thereof, thereby modulating production of Aβ in a tissue of the subject. In some embodiments, the Aβ disorder is Alzheimer's disease.

In one embodiment, the present disclosure provides a method of treating or preventing the progression of Alzheimer's disease and/or related tauopathies in a subject, the method comprising administering to the subject a therapeutically effective amount of at least one myelin sheath protein and/or a synthetic polypeptide comprising a structure similar to said protein, and/or a homolog thereof, and/or a fragment thereof, in combination with at least one agent selected from an inhibitor of tau aggregation, an anti-amyloid antibody, an anti-amyloid aggregation inhibitor, an antibody that binds tau, an antibody that binds beta amyloid, an antibody that binds amyloid-precursor-protein, an antibody that binds amyloid beta-derived diffusible ligands, and an antibody that binds a marker of Alzheimer's disease.

In one embodiment, the present disclosure provides a method of reducing the decline in functional or cognitive capacity in a subject diagnosed with early or mild to moderate Alzheimer's disease (AD) comprising administering to the subject suffering from early or mild to moderate AD a therapeutically effective amount of at least one myelin sheath protein and/or a synthetic polypeptide comprising a structure similar to said protein, and/or a homolog thereof, and/or a fragment thereof, in combination with at least one agent selected from an anti-amyloid antibody, an anti-tau antibody, an antibody that binds beta amyloid, an antibody that binds amyloid-precursor-protein, an antibody that binds amyloid beta-derived diffusible ligands, and an antibody that binds a marker of Alzheimer's disease, in an amount effective to slow the decline in functional or cognitive capacity in the subject.

In one embodiment, the present disclosure provides a method of reducing plaque formation in the brain of a subject, the method comprising administering to the subject a therapeutically effective amount of at least one myelin sheath protein and/or a synthetic polypeptide comprising a structure similar to said protein, and/or a homolog thereof, and/or a fragment thereof, in combination with at least one agent selected from an antibody or antigen-binding fragment thereof that specifically binds beta amyloid, and/or amyloid-precursor-protein, and/or amyloid beta-derived diffusible ligands, and/or tau, and/or a fragment thereof, and/or any combination thereof.

In one embodiment, the present disclosure provides a method of lessening the severity of Alzheimer's disease in the brain of a subject comprising administering a myelin sheath protein and/or a synthetic polypeptide comprising a structure similar to said protein, and/or a homolog thereof, and/or a fragment thereof, and an antibody or antigen-binding fragment thereof that binds to beta amyloid, and/or amyloid-precursor-protein, and/or amyloid beta-derived diffusible ligands and/or an antibody or antigen-binding fragment thereof that binds to tau, and/or a fragment thereof, to a subject having or at risk of developing the disease, thereby lessening the severity of the disease.

In one embodiment, the present disclosure provides a method of monitoring the response of a subject being treated for neurological damage by administering neuroprotective agents, comprising the steps of: (a) determining the amount of at least one biomarker in a first biological sample taken from the subject prior to an initial treatment with a neuroprotective agent, wherein the neuroprotective agent comprises a combination of a myelin sheath protein and/or a synthetic polypeptide comprising a structure similar to said protein, and/or a homolog thereof, and/or a fragment thereof, and an antibody or antigen-binding fragment thereof that specifically binds tau, and/or beta amyloid, and/or amyloid precursor protein, and/or amyloid beta derived diffusible ligands, and/or a fragment thereof, and/or a combination thereof; (b) determining the amount of the biomarker in at least a second biological sample taken from the subject subsequent to the initial treatment with the neuroprotective agent; and (c) comparing the amount of the biomarker in the second biological sample with the amount of the biomarker in the first biological sample; such that a detectable reduction in the amount of the biomarker in the second biological sample compared to the amount of biomarker in the first biological sample indicates that the subject is responding positively to the treatment with the neuroprotective agent.

In one embodiment, the present disclosure provides a method of treating Alzheimer's disease, comprising administering to a subject in need thereof a pharmaceutical composition comprising an effective amount of an antibody or antigen-binding fragment thereof that binds to soluble beta amyloid (Abeta) in combination with a myelin sheath protein and/or a synthetic polypeptide comprising a structure similar to said protein, and/or a homolog thereof, and/or a fragment thereof, wherein the antibody is administered peripherally to the subject to exert its beneficial effects. In some embodiments, the method further comprises administering an antibody or antigen-binding fragment thereof that binds tau and/or a fragment thereof.

In some embodiments, the antibody binds at least one marker selected from the group consisting of P-S396-tau, P-T181-tau, Aβ, Aβ1-42, Aβ1-40, amyloid precursor protein (APP), amyloid precursor protein beta (APPβ), and amyloid precursor protein alpha (APPalpha).

In some embodiments, the biomarker is selected from the group comprising S-100b, neuron-specific enolase (NSE), glial fibrillary acidic protein (GFAP), tau protein, haptoglobin, brain creatine kinase, isoprostane, myelin basic protein (MBP), myelin oligodendrocyte glycoprotein (MOG), beta amyloid, amyloid-precursor-protein, amyloid beta-derived diffusible ligands, thrombomodulin, or a fragment thereof, or any combination thereof.

In some embodiments, the decline in cognitive capacity is assessed by determining the patient's score before and after administration of the antibody using a 12-item Alzheimer's disease Assessment Scale-Cognition (ADAS-Cog12), 13-item Alzheimer's disease Assessment Scale-Cognition (ADAS-Cog13), or 14-item Alzheimer's disease Assessment Scale-Cognition (ADAS-Cog14) test, optionally wherein the reduction in cognitive decline as measured by ADAS-Cog is at least 30%, at least 35%, at least 40%, or at least 45% relative to placebo.

In some embodiments, the neurological disorder is Parkinson's Disease, Alzheimer's disease, amyotrophic lateral sclerosis, stroke, a neuromuscular disorder, schizophrenia, cerebral infarction, head trauma, glaucoma, facialis, or Huntington's Disease. In some embodiments, the neurological damage is a condition or disease, or is caused by a condition, disease or event, selected from the group consisting of cerebral ischemia, cerebral infarction, head trauma, contusion, spinal cord injury, subarachnoid hemorrhage, cerebral hemorrhage, aneurysmal hemorrhage, cardiac infarction, hypoxia, anoxia, surgery, Alzheimer's disease, Parkinson's disease, multiple sclerosis, HIV-related neurodegeneration, cerebellar degeneration, seizure, and ataxia.

In some embodiments, the myelin sheath protein and/or the synthetic polypeptide comprising a structure similar to said protein, and/or a homolog thereof, and/or a fragment thereof is selected from the group consisting of myelin basic protein (MBP) or a fragment thereof, myelin oligodendrocyte glycoprotein (MOG) or a fragment thereof, MBP-85-99, MOG-35-55, glatiramer acetate, or a copolymer 1 related peptide. In some embodiments, the synthetic polypeptide is at least about 80%, at least about 85%, at least about 90%, at least about 95%, at least about 97%, at least about 98%, or at least about 99% identical to a myelin sheath protein or a fragment thereof.

In some embodiments, the myelin sheath protein and/or the synthetic polypeptide comprising a structure similar to said protein, and/or a homolog thereof, and/or a fragment thereof, comprises, consists essentially of, or consists of an amino acid sequence selected from the group consisting of: Asp-Glu-Asn-Pro-Val-Val-His-Phe-Phe-Lys-Asn-Ile-Val-Thr-Pro-Arg-Thr (SEQ ID NO:17); Lys-Ser-His-Gly-Arg-Thr-Gln-Asp-Glu-Asn-Pro-Val-Val-His-Phe-Phe-Lys-Asn-Ile-Val-Thr (SEQ ID NO: 18); Ala-Arg-Thr-Ala-His-Tyr-Gly-Ser-Leu-Pro-Gln-Lys-Ser-His-Gly (SEQ ID NO: 19); His-His-Pro-Ala-Arg-Thr-Ala-His-Tyr-Gly-Ser-Leu-Pro-Gln-Lys (SEQ ID NO: 20); Tyr-Gly-Ser-Leu-Pro-Gln-Lys-Ser-His-Gly-Arg-Thr-Gln-Asp-Glu (SEQ ID NO: 21); Thr-Gln-Asp-Glu-Asn-Pro-Val-Val-His-Phe-Phe-Lys-Asn-Ile-Val-Thr-Pro-Arg (SEQ ID NO: 22); Lys-Asn-Ile-Val-Thr-Pro-Arg-Thr-Pro-Pro-Pro-Ser-Gln-Gly-Lys-Gly (SEQ ID NO: 23); Asn-Pro-Val-Val-His-Phe-Phe-Lys-Asn-Ile (SEQ ID NO: 24); Pro-Val-Val-His-Phe-Phe-Lys-Asn-Ile-Val (SEQ ID NO: 25); Val-Val-His-Phe-Phe-Lys-Asn-Ile-Val-Thr (SEQ ID NO: 26); Val-His-Phe-Phe-Lys-Asn-Ile-Val-Thr-Pro (SEQ ID NO: 27); and Glu-Ala-Tyr-Lys-Ala-Ala-Glu-Lys-Ala-Tyr-Ala-Ala-Lys-Glu-Ala-Ala-Lys-Glu-Ala-Ala-Lys-Ala-Lys-Ala-Glu-Lys-Lys-Ala-Ala-Tyr-Ala-Lys-Ala-Lys-Ala-Ala-Lys-Tyr-Glu-Lys-Lys-Ala-Lys-Lys-Ala-Ala-Ala-Glu-Tyr-Lys-Lys-Lys (SEQ ID NO: 28).

In some embodiments, the antibody or antigen-binding fragment thereof that specifically binds tau binds to monomeric tau, oligomeric tau, non-phosphorylated tau, extracellular tau, and/or phosphorylated tau. In some embodiments, the antibody or antigen-binding fragment thereof that specifically binds tau binds an epitope within amino acids 2 to 24 of mature human tau. In some embodiments, the antibody or antigen-binding fragment thereof that specifically binds tau is selected from the group comprising BMS-986168, C2N-8E12, AADVAC-1, AADVAC-2, ACI-35, RG7345, TRx-237-015 (LMTX), AV-1451, AV-680, Posiphen, or any combination thereof.

In some embodiments, the antibody or antigen-binding fragment thereof that specifically binds beta amyloid is capable of binding oligomeric and/or monomeric forms of beta amyloid. In some embodiments, the antibody or antigen-binding fragment thereof that specifically binds beta amyloid, and/or amyloid-precursor-protein, and/or amyloid beta-derived diffusible ligands, is selected from the group comprising LY2062430, RN-1219, R-1450, humanized m266, or any combination thereof. In some embodiments, the antibody or antigen-binding fragment thereof that specifically binds beta amyloid, and/or amyloid-precursor-protein, and/or amyloid beta-derived diffusible ligands, binds Abeta 42. In some embodiments, the antibody or antigen-binding fragment thereof that specifically binds beta amyloid, and/or amyloid-precursor-protein, and/or amyloid beta-derived diffusible ligands thereof, binds Abeta 40. In some embodiments, the antibody or antigen-binding fragment thereof that specifically binds beta amyloid, and/or amyloid-precursor-protein, and/or amyloid beta-derived diffusible ligands, binds an intracellular Abeta.

In some embodiments, the subject is a human. In some embodiments, the subject has mild cognitive impairment. In some embodiments, the subject has a genomic mutation in the APP gene. In some embodiments, the subject has a genomic mutation in the ApoE gene. In some embodiments, the subject has a genomic mutation in a presenilin gene. In some embodiments, the subject has Alzheimer's disease or a genetic predisposition for developing Alzheimer's disease. In some embodiments, the Alzheimer's disease is sporadic or non-hereditary Alzheimer's disease. In some embodiments, the Alzheimer's disease is familial or hereditary Alzheimer's disease. In some embodiments, the Alzheimer's disease is clinical, pre-clinical, or prodromal Alzheimer's disease. In some embodiments, the subject has familial, and/or sporadic, and/or idiopathic Alzheimer's disease, and/or cerebral amyloid angiopathy. In some embodiments, the subject has amyloid deposits. In some embodiments, the subject's brain has amyloid-beta amyloid deposits. In some embodiments, the subject is at risk of developing Alzheimer's disease. In some embodiments, the subject is selected from the group consisting of subjects with mild cognitive impairment, subjects with genotypes known to be associated with Alzheimer's disease, subjects with Trisomy 21, and subjects with surrogate markers indicating risk for Alzheimer's disease. In some embodiments, the subjects with genotypes known to be associated with Alzheimer's disease comprise subjects with the ApoE4 genotype.

In some embodiments, the antibody comprises an immunoglobulin domain. In some embodiments, the antibody is a monoclonal antibody or a binding fragment thereof. In some embodiments, the antibody is a chimeric antibody or a fragment thereof. In some embodiments, the monoclonal antibody or a binding fragment thereof is a human antibody, a humanized antibody, or a mouse antibody. In some embodiments, the monoclonal antibody or a binding fragment thereof inhibits formation of amyloid deposits in the subject. In some embodiments, the monoclonal antibody or a binding fragment thereof is an IgG class antibody. In some embodiments, the IgG class antibody is selected from the group consisting of IgG1, IgG2, IgG3, IgG4, and IgGM. In some embodiments, the antibody or antigen-binding fragment thereof is a bispecific antibody or an antigen-binding fragment having two antigen-binding regions. In some embodiments, one of the antigen-binding regions of the bispecific antibody or of the antigen-binding fragment having two antigen-binding regions binds to human tau or a fragment thereof. In some embodiments, one of the antigen-binding regions of the bispecific antibody or of the antigen-binding fragment having two antigen-binding regions binds to human amyloid protein or a fragment thereof. In some embodiments, the first antigen-binding region of the bispecific antibody or of the antigen-binding fragment having two antigen-binding regions binds to beta amyloid, and/or amyloid-precursor-protein, and/or amyloid beta-derived diffusible ligands, and/or a fragment thereof, and the second antigen-binding region of the bispecific antibody or of the antigen-binding fragment having two antigen-binding regions binds to human tau and/or a fragment thereof. In some embodiments, the bispecific antibody or the antigen-binding fragment having two antigen-binding regions thereof comprises a first specificity towards: a) an epitope of beta-amyloid peptide that contains residues 28-35 of beta-amyloid; b) an epitope of beta-amyloid peptide that contains residues 28-34 of beta-amyloid; c) an epitope of beta-amyloid peptide that contains residues 28-33 of beta-amyloid; and/or d) an epitope within the region of residues 28-35 of beta-amyloid; and a second specificity towards a tau protein and/or a fragment thereof. In some embodiments, the antibody or the antigen-binding fragment thereof specifically binds to beta amyloid, and/or amyloid-precursor-protein, and/or amyloid beta-derived diffusible ligands, and/or a fragment thereof, and to tau, and/or a fragment thereof. In some embodiments, the antibody or the antigen-binding fragment thereof comprises an antibody or an antigen-binding fragment thereof that specifically binds beta amyloid, and/or amyloid-precursor-protein, and/or amyloid beta-derived diffusible ligands, and an antibody or an antigen-binding fragment thereof that specifically binds tau, and/or a fragment thereof.

In some embodiments, the administration of myelin sheath protein and/or a synthetic polypeptide comprising a structure similar to said protein, and/or a homolog thereof, and/or a fragment thereof, and an antibody or antigen-binding fragment thereof that binds tau, and/or beta amyloid, and/or amyloid-precursor-protein, and/or amyloid beta-derived diffusible ligands, and/or fragments thereof results in a synergistic effect. In some embodiments, the administration of a myelin sheath protein and/or a synthetic polypeptide comprising a structure similar to said protein, and at least one of the antibody or antigen-binding fragment thereof of the present disclosure results in a synergistic effect. In some embodiments, the administration of a myelin sheath protein and/or a synthetic polypeptide comprising a structure similar to said protein, and an antibody or antigen-binding fragment thereof of the present disclosure results in a synergistic effect. In some embodiments, the myelin sheath protein and/or a synthetic polypeptide comprising a structure similar to said protein, and/or a homolog thereof, and/or a fragment thereof is administered before, after, or simultaneously with the antibody or antigen-binding fragment thereof of the present disclosure. In some embodiments, the antibody or antigen-binding fragment thereof can be one or more than one antibody or antigen binding fragment thereof. In some embodiments, the antibody or antigen-binding fragment thereof binds to beta amyloid, and/or amyloid-precursor-protein, and/or amyloid beta-derived diffusible ligands, and/or tau, and/or fragments thereof results.

In some embodiments, the administration is by injection, or by infusion. In some embodiments, the administration is subcutaneous, intramuscular, intravenous, or intradermal.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the descriptions is to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the present disclosure. It should be understood that various alternatives to the embodiments of the disclosure described herein may be employed.

Unless defined otherwise, all technical and scientific terms used herein have the same meaning as commonly understood by one of ordinary skill in the art to which the present disclosure belongs. In case of conflict, the present application including the definitions will control. Also, unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference, unless only specific sections of patents or patent publications are indicated to be incorporated by reference.

In order to further define the present disclosure, the following terms, abbreviations and definitions are provided.

As used herein, the term “about” modifying the quantity of an ingredient or agent (e.g., active agent) or compound or antibody of the present disclosure employed refers to variations in the numerical quantity that may occur, for example, through typical measuring and handling procedures (e.g., liquid handling procedures) used for making concentrates or compositions or solutions in the real world; through inadvertent error in these procedures; through differences in the manufacture, source, or purity of the ingredients employed to make the compositions or to carry out the methods; and the like. The term “about” also encompasses amounts that differ due to different equilibrium conditions for a composition resulting from a particular initial mixture. Whether or not modified by the term “about”, the claims include equivalents to the quantities. In some embodiments, the term “about” means within 10% of the reported numerical value, or within 2% of the reported numerical value, or within 5% of the reported numerical value, or within 20% of the reported numerical value.

The articles “a,” “an,” and “the” preceding an element or component of the present disclosure are intended to be nonrestrictive regarding the number of instances, i.e., occurrences of the element or component. Therefore “a” or “an” or “the” should be read to include one or at least one, and the singular word form of the element or component also includes the plural unless the number is obviously meant to be singular.

As used herein, “homologue” refers to a protein or peptide or polypeptide that is functionally equivalent, but may have a limited number of amino acid substitutions, deletions, insertions or additions in the amino acid sequence. In order to maintain the function of the protein or peptide or polypeptide, the substitutions may be conservative substitutions, replacing an amino acid with one having similar properties.

In some embodiments, a homologue refers to a protein or peptide or polypeptide which has an identity of at least 25%, at least 50%, at least 60%, at least 70%, at least 80%, at least 85%, at least 90%, at least 95%, or at least 99% with the amino acid of the native or isolated protein or peptide or polypeptide, and/or with the amino acid sequence relative to a SEQ ID NO corresponding to the protein or peptide or polypeptide. Algorithms for determining sequence identity are publicly available and include e.g. BLAST available through the National Center for Biotechnology Information (NCBI). One skilled in the art may determine if the sequences are similar to a degree that indicates homology and thus similar or identical function.

As used herein, “synergistic” refers to a greater-than-additive effect produced by a combination (i.e., an effect that is greater than the sum of individual effects) or an additive effect when the individual effects are not expected to be additive. The term also refers to the addition of one agent which results in less of another agent being required.

In some embodiments two or more agents may be administered simultaneously or sequentially, and may act synergistically to modify or ameliorate disease progression or symptoms or to treat a disease. In some embodiments, the combination of the first agent of the present disclosure with a second (therapeutic) agent and/or a third or more agents produces an enhanced therapeutic profile, for example, a profile that is greater than the sum of the benefits of the treatment with each agent independently. In some embodiments, the first agent and/or the second agent and/or the third or more agents, when simultaneously present in a subject, act synergistically to reduce, inhibit, or ameliorate the symptoms or pathogenesis of a disease, e.g., Alzheimer's disease. In some embodiments, the first agent and/or the second agent and/or the third or more agents, when simultaneously present in a subject, act synergistically to reduce, inhibit, or ameliorate the symptoms or pathogenesis of a neurodegenerative disease, and/or a tauopathy, and/or an amyloidosis, and/or an amyloid disease, and/or an AP disorder. In some embodiments, the first agent and/or the second agent and/or the third or more agents, when administered to a subject, act synergistically to reduce, inhibit, or ameliorate the symptoms or pathogenesis of a neurodegenerative disease, and/or a tauopathy, and/or an amyloidosis, and/or an amyloid disease, and/or an AP disorder. In some embodiments, the administration can be simultaneous, or sequentially. In some embodiments, the administration of one agent can be hours or days before the administration of the second and/or third or more agents. In some embodiments, the first agent is a myelin sheath protein and/or a synthetic polypeptide comprising a structure similar to said protein, and/or a homolog thereof, and/or a fragment thereof. In some embodiments, the second agent is an antibody or antigen-binding fragment thereof that specifically binds beta amyloid, and/or amyloid-precursor-protein, and/or amyloid beta-derived diffusible ligands, and/or tau, and/or a fragment thereof, and/or any combination thereof. In some embodiments, the second agent comprises at least one antibody or antigen-binding fragment thereof. In some embodiments, the second agent is an antibody or antigen-binding fragment thereof that specifically binds beta amyloid, and/or amyloid-precursor-protein, and/or amyloid beta-derived diffusible ligands, and/or a fragment thereof. In some embodiments, the second agent is an antibody or antigen-binding fragment thereof that specifically binds tau, and/or a fragment thereof. In some embodiments, the second agent comprises at least two antibodies or antigen-binding fragments thereof. In some embodiments, one of the at least two antibodies or antigen-binding fragments thereof binds to beta amyloid, and/or amyloid-precursor-protein, and/or amyloid beta-derived diffusible ligands, and/or fragments thereof. In some embodiments, one of the at least two antibodies or antigen-binding fragment thereof binds to tau and/or a fragment thereof. In some embodiments, the second agent is a bispecific antibody. In some embodiments, the bispecific antibody binds to beta amyloid, and/or amyloid-precursor-protein, and/or amyloid beta-derived diffusible ligands, and/or fragments thereof, and binds to tau, and/or a fragment thereof. In some embodiments both the second agent and the third agent is an antibody or an antigen-binding fragment thereof. In some embodiments, a third agent is a compound or an agent that is commonly used (e.g., compounds and agents known in the art and/or compounds and agents disclosed in the present disclosure) to treat a disorder and/or ameliorate a symptom or progression of a disorder disclosed in the present disclosure. In some embodiments, a second, or a third, or a fourth or more agents are administered. In some embodiments, the combination of a first and second agent provides unexpected and/or superior results compared to the administration of the first agent alone or the second agent alone. In some embodiments, the combination of a first, second, and third agents provides unexpected and/or superior results compared to the administration of the first agent alone or the second agent alone or the third agent alone and/or provides unexpected and/or superior results compared to the administration of any combination of two agents (e.g., first and second agent; second and third agent; first and third agent). In some embodiments, the administration is simultaneously. In some embodiments, the administration is sequentially. In some embodiments, the one or more agents are administered separately (e.g., the first agent is administered separately from the second or more agents). In some embodiments, the one or more agents are administered together or at the same time or within a few minutes from each other. In some embodiments, the agents are in separate compositions and/or pharmaceutical formulations. In some embodiments, the agents (e.g., one or more agents, such as the first and second agent and so on) are present in the same composition and/or pharmaceutical formulation.

As used herein, the term “neurological disorder” can include diseases and disorders of the central nervous system (e.g., the brain and spinal cord). Examples of such neurological disorder can include, but is not limited to, Alzheimer's disease, attention deficit hyperactivity disorder, epilepsy, head trauma, schizophrenia, brain cancer, stroke, parkinson's disease, amyotrophic lateral sclerosis, neuromuscular disorder, cerebral infarction, head trauma, glaucoma, facialis, and Huntington's disease.

The term “tauopathy” refers to tau-related disorders or conditions, e.g., Alzheimer's Disease, Progressive Supranuclear Palsy (PSP), Corticobasal Degeneration (CBD), Pick's Disease, Frontotemporal dementia and Parkinsonism associated with chromosome 17 (FTDP-17), Parkinson's disease, stroke, traumatic brain injury, mild cognitive impairment, and the like. A tauopathy is a disorder characterized by an abnormal level of tau in a tissue or fluid in an individual. In some cases, a tauopathy is characterized by the presence in a tissue or a fluid of elevated (higher than normal) levels of tau or tau peptides and/or pathological forms of tau. For example, in some cases, a tauopathy is characterized by the presence in brain tissue and/or cerebrospinal fluid of elevated levels of tau or tau peptides and/or pathological forms of tau. A “higher than normal” level of tau in a tissue or a fluid indicates that the level of tau in the tissue or fluid is higher than a normal, control level, e.g., higher than a normal, control level for an individual or population of individuals of the same age group. See, e.g., Blomberg et al. (2001) “Cerebrospinal fluid tau levels increase with age in healthy individuals” Dement. Geriatr. Cogn. Disord. 12: 127. In some cases, an individual having a tauopathy exhibits one or more additional symptoms of a tauopathy (e.g., cognitive decline). In other cases, a tauopathy is characterized by the presence in a tissue or a fluid of lower than normal levels of tau. A “lower than normal” level of tau in a tissue or a fluid indicates that the level of tau in the tissue or fluid is lower than a normal, control level, e.g., lower than a normal, control level for an individual or population of individuals of the same age group. In some embodiments, a tauopathy is Alzheimer's, Pick's disease, sporadic Frontotemporal dementia, and/or Frontotemporal dementia with Parkinsonism linked to chromosome 17. Other tauopathies include but are not limited to Progressive supranuclear palsy (PSP), Corticobasal degeneration (CBD) and Subacute sclerosing panencephalitis. A neurodegenerative tauopathy includes, but is not limited to, Alzheimer's disease, amyotrophic lateral sclerosis/parkinsonism-dementia complex, argyrophilic grain dementia, British type amyloid angiopathy, cerebral amyloid angiopathy, corticobasal degeneration, Creutzfeldt-Jakob disease, dementia pugilistica, diffuse neurofibrillary tangles with calcification, Down's syndrome, frontotemporal dementia, frontotemporal dementia with parkinsonism linked to chromosome 17, frontotemporal lobar degeneration, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, subacute sclerosing panencephalitis, Tangle only dementia, multi-infarct dementia, ischemic stroke, chronic traumatic encephalopathy (CTE), traumatic brain injury (TBI), and stroke.

An “anti-tau antibody” refers to an antibody that binds a tau peptide. In some embodiments, an anti-tau antibody binds a tau peptide and/or a fragment thereof. In some embodiments, an anti-tau antibody binds human tau, and/or human tau protein, and/or human tau splice variants, and/or a tau epitope, and/or a mature human tau. In some embodiments, an anti-tau antibody binds a tau polypeptide with a Kd less than about 10⁻⁷, less than about 10⁻⁸, less than about 10⁻⁹, less than about 10⁻¹⁰, less than about 10⁻¹¹, or less than about 10¹²M, or less.

“Specific binding” or “specifically binds” or “binds” or “anti-target antibody” or “an antibody that binds to” refers to an antibody binding to an antigen or an epitope within the antigen with greater affinity than for other antigens. In some embodiments, the extent of binding of an anti-target antibody to an unrelated, non-target protein is less than about 10% of the binding of the antibody to target as measured, e.g., by a radioimmunoassay (RIA) or biacore assay. In some embodiments, an antibody that binds to a target has a dissociation constant (K_{D}) of less than or equal to 1 micromolar, less than or equal to 100 nM, less than or equal to 10 nM, less than or equal to 1 nM, less than or equal to 0.1 nM, less than or equal to 0.01 nM, or less than or equal to 0.001 nM. In some embodiments, an anti-target antibody binds to an epitope of a target that is conserved among different species. Typically, the antibody binds to the antigen or the epitope within the antigen with an equilibrium dissociation constant (K_{D}) of about 1×10⁻⁸M or less, for example about 1×10⁻⁹M or less, about 1×10⁻¹⁰ M or less, about 1×10⁻¹¹ M or less, or about 1×10⁻¹² M or less, typically with the K_{D} that is at least one hundred fold less than its K_{D} for binding to a non-specific antigen (e.g., BSA, casein). The dissociation constant may be measured using standard procedures. Antibodies that specifically bind to the antigen or the epitope within the antigen may, however, have cross-reactivity to other related antigens, for example to the same antigen from other species (homologs), such as human, mouse, rat or monkey, for example Macaca fascicularis (cynomolgus, cyno), Pan troglodytes (chimpanzee, chimp) or Callithrix jacchus (common marmoset, marmoset). While a monospecific antibody specifically binds one antigen or one epitope, a bispecific antibody specifically binds two distinct antigens or two distinct epitopes.

“Anti-Abeta immunoglobulin,” “anti-Abeta antibody,” “antibody that binds Abeta”, and “antibody that binds beta amyloid” are used interchangeably herein, and refer to an antibody that specifically binds to Abeta (e.g., human Abeta). A nonlimiting example of an anti-Abeta antibody is crenezumab. Other non-limiting examples of anti-Abeta antibodies are solanezumab, bapineuzumab, aducanumab, and gantenerumab.

The terms “antibody” and “immunoglobulin” (“Ig”) are used interchangeably in the broadest sense and include, but are not limited to, monoclonal antibodies (for example, full length or intact monoclonal antibodies), polyclonal antibodies, multivalent antibodies, antibodies with polyepitopic specificity, single chain antibodies, multi-specific antibodies (for example, bispecific antibodies, trispecific antibodies, tetraspecific antibodies), and fragments of antibodies, provided they exhibit the desired biological activity. Such antibodies can be chimeric, humanized, human, synthetic, and/or affinity matured. Such antibodies and methods of generating them are described in more detail herein. “Antibodies” is meant in a broad sense and includes immunoglobulin molecules including monoclonal antibodies including murine, human, humanized and chimeric monoclonal antibodies, antigen-binding fragments, bispecific or multispecific antibodies, dimeric, tetrameric or multimeric antibodies, single chain antibodies, domain antibodies and any other modified configuration of the immunoglobulin molecule that comprises an antigen binding site of the required specificity. “Full length antibody molecules” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Complementarity determining regions (CDR)” are “antigen binding sites” in an antibody. CDRs may be defined using various terms: (i) Complementarity Determining Regions (CDRs), three in the VH (HCDR1, HCDR2, HCDR3) and three in the VL (LCDR1, LCDR2, LCDR3) are based on sequence variability (Wu and Kabat, (1970) J Exp Med 132:211-50; Kabat et al., Sequences of Proteins of Immunological Interest, 5th Ed. Public Health Service, National Institutes of Health, Bethesda, Md., 1991). (ii) “Hypervariable regions”, “HVR”, or “HV”, three in the VH (H1, H2, H3) and three in the VL (L1, L2, L3) refer to the regions of an antibody variable domains which are hypervariable in structure as defined by Chothia and Lesk (Chothia and Lesk, (1987) Mol Biol 196:901-17). The International ImMunoGeneTics (IMGT) database (http://www_imgt_org) provides a standardized numbering and definition of antigen-binding sites. The correspondence between CDRs, HVs and IMGT delineations is described in Lefranc et al., (2003) Dev Comparat Immunol 27:55-77. The term “CDR”, “HCDR1”, “HCDR2”, “HCDR3”, “LCDR1”, “LCDR2” and “LCDR3” as used herein includes CDRs defined by any of the methods described herein, Kabat, Chothia or IMGT, unless otherwise explicitly stated otherwise.

Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may assigned to one of two clearly distinct types, namely kappa (κ) and lambda (λ), based on the amino acid sequences of their constant domains.

“Antigen-binding fragment” refers to a portion of an immunoglobulin molecule that retains the antigen binding properties of the parental full length antibody. Exemplary antigen-binding fragments are heavy chain complementarity determining regions (HCDR) 1, 2 and/or 3, light chain complementarity determining regions (LCDR) 1, 2 and/or 3, a heavy chain variable region (VH), or a light chain variable region (VL), Fab, F(ab')₂, Fd and Fv fragments as well as domain antibodies (dAb) consisting of either one VH domain or one VL domain. VH and VL domains may be linked together via a synthetic linker to form various types of single chain antibody designs in which the VH/VL domains pair intramolecularly, or intermolecularly in those cases when the VH and VL domains are expressed by separate chains, to form a monovalent antigen binding site, such as single chain Fv (scFv) or diabody; described for example in Int. Pat. Publ. No. WO1998/44001, Int. Pat. Publ. No. WO1988/01649; Int. Pat. Publ. No. WO1994/13804; Int. Pat. Publ. No. WO1992/01047.

“Antibody fragments” comprise only a portion of an intact antibody, wherein the portion preferably retains at least one, and typically most or all, of the functions normally associated with that portion when present in an intact antibody. In some embodiments, an antibody fragment comprises an antigen binding site of the intact antibody and thus retains the ability to bind antigen. In some embodiments, an antibody fragment, for example one that comprises the Fc region, retains at least one of the biological functions normally associated with the Fc region when present in an intact antibody, such as FcRn binding, antibody half life modulation, ADCC function and complement binding. In some embodiments, an antibody fragment is a monovalent antibody that has an in vivo half life substantially similar to an intact antibody. For example, such an antibody fragment may comprise an antigen binding arm linked to an Fc sequence capable of conferring in vivo stability to the fragment. Examples of antibody fragments include but are not limited to Fv, Fab, Fab', Fab'-SH, F(ab')₂; diabodies; linear antibodies; single-chain antibody molecules (e.g. scFv); and multispecific antibodies formed from antibody fragments. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab')₂fragments comprising salvage receptor binding epitope residues and having increased in vivo half-life, see U.S. Pat. No. 5,869,046.

“Monoclonal antibody” refers to an antibody population with single amino acid composition in each heavy and each light chain, except for possible well known alterations such as removal of C-terminal lysine from the antibody heavy chain, and intentionally made asymmetrical substitutions into the heavy chains for example to promoter heterodimer formation when generating bispecific full length antibodies, or to facilitate purification of antibodies using protein A columns. Monoclonal antibodies typically bind one antigenic epitope, except that bispecific monoclonal antibodies bind two distinct antigenic epitopes. Monoclonal antibodies may have heterogeneous glycosylation within the antibody population. Monoclonal antibody may be monospecific or multispecific, or monovalent, bivalent or multivalent. A bispecific antibody is included in the term monoclonal antibody.

“Isolated” refers to a homogenous population of molecules (such as synthetic polynucleotides or proteins or peptides or antibodies) which have been substantially separated and/or purified away from other components of the system the molecules are produced in, such as a recombinant cell, as well as a protein that has been subjected to at least one purification or isolation step. “Isolated antibody” refers to an antibody that is substantially free of other cellular material and/or chemicals and encompasses antibodies that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% pure.

“Humanized antibody” refers to an antibody in which the antigen binding sites are derived from non-human species and the variable region frameworks are derived from human immunoglobulin sequences. Humanized antibody may include substitutions in the framework so that the framework may not be an exact copy of expressed human immunoglobulin or human immunoglobulin germline gene sequences.

“Human antibody” refers to an antibody having heavy and light chain variable regions in which both the framework and the antigen binding site are derived from sequences of human origin. If the antibody contains a constant region or a portion of the constant region, the constant region also is derived from sequences of human origin.

Human antibody comprises heavy or light chain variable regions that are “derived from” sequences of human origin if the variable regions of the antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci as described herein. “Human antibody” may contain amino acid differences when compared to the human germline immunoglobulin or rearranged immunoglobulin genes due to for example naturally occurring somatic mutations or intentional introduction of substitutions into the framework or antigen binding site, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98%, 99% or 100% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462.

Human antibodies derived from human immunoglobulin sequences may be generated using systems such as phage display incorporating synthetic CDRs and/or synthetic frameworks, or may be subjected to in vitro mutagenesis to improve antibody properties, resulting in antibodies that are not expressed by the human antibody germline repertoire in vivo.

Antibodies in which antigen binding sites are derived from a non-human species are not included in the definition of “human antibody”.

“Recombinant” refers to antibodies and other proteins that are prepared, expressed, created or isolated by recombinant means.

“Epitope” refers to a portion of an antigen to which an antibody specifically binds. Epitopes typically consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule. Antibody “epitope” depends on the methodology used to identify the epitope.

“Multispecific” refers to an antibody that specifically binds at least two distinct antigens or two distinct epitopes within the antigens, for example three, four or five distinct antigens or epitopes.

“Bispecific” refers to an antibody that specifically binds two distinct antigens or two distinct epitopes within the same antigen. Bispecific antibody may have cross-reactivity to other related antigens, for example to the same antigen from other species (homologs), such as human or monkey, for example Macaca fascicularis (cynomolgus, cyno), Pan troglodytes (chimpanzee, chimp) or Callithrix jacchus (common marmoset, marmoset), or may bind an epitope that is shared between two or more distinct antigens.

“Bispecific anti-tau/beta amyloid antibody”, “tau/beta amyloid antibody”, “anti-tau/beta amyloid antibody” or “antibody that specifically binds tau and beta amyloid” refers to a molecule comprising at least one domain specifically binding tau and at least one domain specifically binding beta amyloid. The bispecific anti-tau/beta amyloid antibody may be monovalent in terms of its binding to either tau or beta amyloid.

“Bispecific anti-tau/amyloid-precursor-protein antibody”, “tau/amyloid-precursor-protein antibody”, “anti-tau/amyloid-precursor-protein antibody” or “antibody that specifically binds tau and amyloid-precursor-protein” refers to a molecule comprising at least one domain specifically binding tau and at least one domain specifically binding amyloid-precursor-protein. The bispecific anti-tau/amyloid-precursor-protein antibody may be monovalent in terms of its binding to either tau or amyloid-precursor-protein.

“Bispecific anti-tau/amyloid beta-derived diffusible ligand antibody”, “tau/amyloid beta-derived diffusible ligand antibody”, “anti-tau/amyloid beta-derived diffusible ligand antibody” or “antibody that specifically binds tau and amyloid beta-derived diffusible ligand” refers to a molecule comprising at least one domain specifically binding tau and at least one domain specifically binding amyloid beta-derived diffusible ligand. The bispecific anti-tau/amyloid beta-derived diffusible ligand antibody may be monovalent in terms of its binding to either tau or amyloid beta-derived diffusible ligand.

“Variant” refers to a polypeptide or a polynucleotide that differs from a reference polypeptide or a reference polynucleotide by one or more modifications, for example one or more substitutions, insertions or deletions.

“Polypeptide” or “protein” refers to a molecule that comprises at least two amino acid residues linked by a peptide bond to form a polypeptide. Small polypeptides of less than 50 amino acids may be referred to as “peptides”.

The term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

“Sample” refers to a collection of similar fluids, cells, or tissues isolated from a subject, as well as fluids, cells, or tissues present within a subject. Exemplary samples are biological fluids such as blood, serum and serosal fluids, plasma, lymph, urine, saliva, cystic fluid, tear drops, feces, sputum, mucosal secretions of the secretory tissues and organs, vaginal secretions, ascites fluids, fluids of the pleural, pericardial, peritoneal, abdominal and other body cavities, fluids collected by bronchial lavage, liquid solutions contacted with a subject or biological source, for example, cell and organ culture medium including cell or organ conditioned medium, lavage fluids and the like, tissue biopsies, fine needle aspirations or surgically resected tumor tissue, or synovial biopsies. The source of the tissue sample may be solid tissue as from a fresh, frozen and/or preserved organ or tissue sample or biopsy or aspirate; blood or any blood constituents; bodily fluids; and cells from any time in gestation or development of the subject or plasma. The term “biological sample” as used herein includes, but is not limited to, blood, serum, plasma, sputum, tissue biopsies (e.g., lung samples), and nasal samples including nasal swabs or nasal polyps.

A “reference sample,” as used herein, refers to any sample, standard, or level that is used for comparison purposes. In some embodiments, a reference sample is obtained from a healthy and/or non-diseased part of the body (e.g., tissue or cells) of the same subject or patient. In some embodiments, a reference sample is obtained from an untreated tissue and/or cell of the body of the same subject or patient. In some embodiments, a reference sample is obtained from a healthy and/or non-diseased part of the body (e.g., tissues or cells) of an individual who is not the subject or patient. In some embodiments, a reference sample is obtained from an untreated tissue and/or cell part of the body of an individual who is not the subject or patient. In some embodiments, a reference sample is a single sample or combined multiple samples from the same subject or patient that are obtained at one or more different time points than when the test sample is obtained. For example, a reference sample is obtained at an earlier time point from the same subject or patient than when the test sample is obtained. In some embodiments, a reference sample includes all types of biological samples as defined above under the term “sample” that is obtained from one or more individuals who is not the subject or patient. In some embodiments, a reference sample is obtained from one or more individuals with a neurological disorder, and/or an amyloid disease, and/or a tauopathy, and/or an Aβ disorder, and/or a disease that causes cognitive impairment, and/or a disease characterized in part by beta amyloid expression activity, activity, or deposition, and/or neurological damage, and/or amyloidosis, e.g., Alzheimer's Disease, who is not the subject or patient. In some embodiments, a reference sample is a combined multiple samples from one or more healthy individuals who are not the subject or patient. In some embodiments, a reference sample is a combined multiple samples from one or more individuals with a disease or disorder (e.g., amyloidosis such as, for example, Alzheimer's Disease) who are not the subject or patient. In some embodiments, a reference sample is pooled RNA samples from normal tissues or pooled plasma or serum samples from one or more individuals who are not the subject or patient.

The phrase “substantially similar,” or “substantially the same,” as used herein, denotes a sufficiently high degree of similarity between two numeric values (generally one associated with an antibody or protein of the present disclosure and the other associated with a reference/comparator antibody or protein) such that one of skill in the art would consider the difference between the two values to be of little or no biological and/or statistical significance within the context of the biological characteristic measured by said values (e.g., Kd values). The difference between said two values is less than about 50%, less than about 40%, less than about 30%, less than about 20%, less than about 10% as a function of the value for the reference/comparator antibody or protein.

The terms “monomeric tau” or “tau monomer,” as used herein, refer to completely solubilized Tau proteins without aggregated complexes in aqueous medium.

The terms “aggregated tau”, “oligomeric tau” and “tau oligomer,” as used herein, refer to multiple aggregated monomers of tau peptides or proteins, or of tau-like peptides/proteins, or of modified or truncated tau peptides/proteins or of other derivates of tau peptides/proteins forming oligomeric or polymeric structures which are insoluble or soluble both in vitro in aqueous medium and in vivo in the mammalian or human body (e.g., the brain). In some embodiments, to multiple aggregated monomers of tau or of modified or truncated tau peptides/proteins or of derivatives thereof, which are insoluble or soluble in the mammalian or human body (e.g., the brain), respectively.

The term “phosphorylated tau” or “pTau,” as used herein, can refer to tau in which a serine, a threonine or a tyrosine residue is phosphorylated by a protein kinase by the addition of a covalently bound phosphate group. In some embodiments, pTau is phosphorylated on a serine or on a threonine residue. In some embodiments, pTau is phosphorylated on Serine at position 409 and/or Serine at position 404. In some embodiments, pTau is phosphorylated on Serine at position 409.

The terms “soluble tau” or “soluble tau protein,” as used herein, refer to proteins consisting of both completely solubilized tau protein/peptide monomers or of tau-like peptides/proteins, or of modified or truncated tau peptides/proteins or of other derivates of tau peptides/proteins monomers, and of tau protein oligomers. “Soluble tau” excludes particularly neurofibrillary tangles (NFT).

The term “insoluble tau,” as used herein, refers to multiple aggregated monomers of tau peptides or proteins, or of tau-like peptides/proteins, or of modified or truncated tau peptides/proteins or of other derivates of tau peptides/proteins forming oligomeric or polymeric structures which are insoluble both in vitro in aqueous medium and in vivo in the mammalian or human body (e.g., in the brain). In some embodiments, to multiple aggregated monomers of tau or of modified or truncated tau peptides/proteins or of derivatives thereof, which are insoluble in the mammalian or human body more particularly in the brain, respectively. “Insoluble Tau” particularly includes neurofibrillary tangles (NFT).

“In combination with” means that two or more therapeutics are administered to a subject together in a mixture, concurrently as single agents or sequentially as single agents in any order.

“Subject” or “patient” or “individual” as used interchangeably includes any human or nonhuman animal. “Nonhuman animal” includes all vertebrates, e.g., mammals and non-mammals, such as nonhuman primates, sheep, dogs, cats, horses, cows chickens, amphibians, reptiles, etc. Exemplary non-human mammals include laboratory, domestic, pet, sport, and stock animals, e.g., mice, cats, dogs, horses, and cows. In some embodiments, the subject is eligible for treatment, e.g., displays one or more indicia of disease. In some embodiments, the subject or patient is eligible for treatment for amyloidosis and/or a neurological disease, e.g., Alzheimer's disease (AD), and/or any disease disclosed herein. In some embodiments, such eligible subject or patient is one that is experiencing or has experienced one or more signs, symptoms, or other indicators of AD or has been diagnosed with AD, whether, for example, newly diagnosed, previously diagnosed or at risk for developing AD. Diagnosis of AD may be made based on clinical history, clinical examination, and established imaging modalities. In some embodiments, a “patient” or “subject” herein includes any single human subject eligible for treatment who is experiencing or has experienced one or more signs, symptoms, or other indicators of AD. Intended to be included as a subject are any subjects involved in clinical research trials, or subjects involved in epidemiological studies, or subjects once used as controls. The subject may have been previously treated with an anti-Abeta antibody, anti-tau antibody, anti-amyloid precursor protein antibody, anti-amyloid beta derived diffusible ligands antibody, or antibody fragments thereof, or antigen-binding fragments thereof, a myelin sheath protein or a synthetic polypeptide comprising a structure similar to said protein, or another drug, or not so treated. In some embodiments, the subject may be naive to an additional drug(s) being used when the treatment herein is started. In some embodiments, the subject may be naive to any agent known in the art used to treat a disease disclosed herein.

“Valent” refers to the presence of a specified number of binding sites specific for an antigen in a molecule. As such, the terms “monovalent”, “bivalent”, “tetravalent”, and “hexavalent” refer to the presence of one, two, four and six binding sites, respectively, specific for an antigen in a molecule.

“Antagonist” refers to a molecule that, when bound to a cellular protein, suppresses at least one reaction or activity that is induced by a natural ligand of the protein. A molecule is an antagonist when the at least one reaction or activity is suppressed by at least about 30%, 40%, 45%, 50%, 55%, 60%, 65%, 70%, 75%, 80%, 85%, 90%, 95%, or 100% more than the at least one reaction or activity suppressed in the absence of the antagonist (e.g., negative control), or when the suppression is statistically significant when compared to the suppression in the absence of the antagonist. Antagonist may be an antibody, a soluble ligand, a small molecule, a DNA or RNA such as siRNA. An exemplary antagonist is an antagonistic bispecific anti-tau/beta amyloid antibody.

The terms “amyloid beta,” “beta amyloid,” “Abeta,” “amyloidβ,” and “Aβ”, used interchangeably herein, refer to the fragment of amyloid precursor protein (“APP”) that is produced upon β-secretase 1 (“BACE1”) cleavage of APP, as well as modifications, fragments and any functional equivalents thereof, including, but not limited to, Aβ1-40, and Aβ1-42. Aβ is known to exist in monomeric form, as well as to associate to form oligomers and fibril structures, which may be found as constituent members of amyloid plaque. The structure and sequences of such Aβ peptides are well known to one of ordinary skill in the art and methods of producing said peptides or of extracting them from brain and other tissues are described, for example, in Glenner and Wong, Biochem Biophys Res. Comm. 129: 885-890 (1984). Moreover, Aβ peptides are also commercially available in various forms. An exemplary amino acid sequence of human A131-42 is DAEFRHDSGYEVHHQKLVFFAEDVGSNKGAIIGLMVGGVVIA (SEQ ID NO: 1). Aβ is peptide of about 39-43 amino acids that corresponds to a peptide formed in vivo upon cleavage of an amyloid beta precursor protein (APP or ABPP) by beta-secretase (at the N-terminal portion of Aβ) and gamma secretase (at the C-terminal portion of Aβ). See, e.g., Strooper and Annaert (2000; J. Cell Set, 113, 1857-1870) and Evin and Weidemann (2002; Peptides, 23, 1285-1297). The most common isoforms of Aβ are Aβ40 and Aβ42, 40 and 42 amino acids, respectively. Aβ42 is less common, but is thought to be more fibrillogenic than Aβ40. Effective antibodies may bind both Aβ40 and Aβ42, selectively bind Aβ42, selectively bind Aβ40, bind all or some isoforms of Aβ, or the like.

The term “amyloidosis,” or amyloid disease, as used herein, refers to a group of diseases and disorders caused by or associated with amyloid or amyloid-like proteins and includes, but is not limited to, diseases and disorders caused by the presence or activity of amyloid-like proteins in monomeric, fibril, or polymeric state, or any combination of the three, including by amyloid plaques. Such diseases include, but are not limited to, secondary amyloidosis and age-related amyloidosis, such as diseases including, but not limited to, neurological disorders such as Alzheimer's Disease (“AD”), diseases or conditions characterized by a loss of cognitive memory capacity such as, for example, mild cognitive impairment (MCI), Lewy body dementia, Down's syndrome, hereditary cerebral hemorrhage with amyloidosis (Dutch type), the Guam Parkinson-Demential complex and other diseases which are based on or associated with amyloid-like proteins such as progressive supranuclear palsy, multiple sclerosis, Creutzfeld Jacob disease, Parkinson's disease, HIV-related dementia, ALS (amyotropic lateral sclerosis), inclusion-body myositis (IBM), adult onset diabetes, endocrine tumor and senile cardiac amyloidosis, and various eye diseases including macular degeneration, drusen- related optic neuropathy, glaucoma, and cataract due to beta-amyloid deposition. Amyloid disease (disorders of protein folding), or amyloidosis, is characterized by the accumulation of a peptide, including the Abeta peptide, existing as abnormal insoluble cross-beta sheet fibrils or amyloid deposits in the affected organs. Amyloid diseases include, but are not limited to, Alzheimer's disease, type 2 diabetes, Huntington's disease, Parkinson's disease, and chronic inflammation.

The term “therapeutic agent” or active agent refers to any agent that is used to treat a disease, including but not limited to an agent that treats a symptom of the disease.

As used herein, “treatment” (and grammatical variations thereof such as “treat” or “treating”) refers to clinical intervention in an attempt to alter the natural course of the individual being treated, and can be performed during the course of clinical pathology. In some embodiments, “treatment” may alleviate a symptom of a disease. In some embodiments, “treatment” may delay the onset of a disease. In some embodiments, “treatment” may delay the progression of a disease. Desirable effects of treatment include, but are not limited to, alleviation or amelioration of one or more symptoms, diminishment of or delay in the appearance of or worsening of any direct or indirect pathological consequences of the disease, decrease of the rate of disease progression, and amelioration or palliation of the disease state. In some embodiments, antibodies and/or a myelin sheath protein or a synthetic polypeptide comprising a structure similar to said protein are used to delay development of a disease or to slow the progression of a disease. In some embodiments, the antibody is an anti-tau antibody and/or an antibody that binds beta amyloid. In some embodiments, the antibody is bispecific to tau and beta amyloid. “Treating” (or “treat” or “treatment”) refers to processes involving a slowing, interrupting, inhibiting, arresting, controlling, stopping, reducing, ameliorating, or reversing the progression, duration, or severity of an existing symptom, disorder, condition, or disease, but does not necessarily involve a total elimination of all disease-related symptoms, conditions, or disorders. Furthermore, “treating”, “treatment” or “treat” refers to an approach for obtaining beneficial or desired results including clinical results, which include, but are not limited to, one or more of the following: inhibiting, delaying or preventing the onset of, or the progression of, a disease associated with beta amyloid activity, or characterized by aberrant deposition of beta amyloid in a subject, such as in Alzheimer's disease; or inhibiting, preventing, or ameliorating at least one symptom associated with a disease associated with beta amyloid activity, or characterized by aberrant deposition of beta amyloid in a subject, such as in Alzheimer's disease, wherein the symptoms include, but are not limited to, cognitive impairment, memory loss, depression, anxiety, dementia, irritability, confusion, mood swings, aggressive and/or apathetic behavior. “Treatment” or “treating”, as used herein, also refers to increasing the quality of life of those suffering from the disease, decreasing the dose of other medications required to treat the disease and/or prolonging survival of patients.

“Treatment regimen” refers to a combination of dosage, frequency of administration, or duration of treatment, with or without addition of a second medication.

“Delaying the onset of” a disease, e.g., Alzheimer's disease, or a symptom thereof means to defer, hinder, slow, retard, stabilize, and/or postpone development of the disease, or a symptom associated with, or resulting from the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of a disease, e.g., Alzheimer's disease, is a method that reduces probability of disease development in a given time frame and/or reduces extent of the disease in a given time frame, when compared to not using the method. Such comparisons are typically based on clinical studies, using a statistically significant number of subjects.

“Prodromal Alzheimer's disease”, also referred to as “predementia stage of AD” refers to the early symptomatic predementia phase of AD in which 1) clinical symptoms including episodic memory loss of the hippocampal type are present, but not sufficiently severe to affect instrumental activities of daily living and do not warrant a diagnosis of dementia; and in which 2) biomarker evidence from CSF or imaging is supportive of the presence of AD pathological changes.

“Preclinical Alzheimer's disease”, which includes both “asymptomatic at-risk state for AD” and “presymptomatic AD” refer to the long asymptomatic stage between the earliest pathogenic events/brain lesions of AD and the first appearance of specific cognitive changes. The “asymptomatic at-risk” state for AD is identified in vivo by evidence of amyloidosis in the brain (with retention of specific PET amyloid tracers) or in the CSF (with changes in amyloid beta, tau, and phosphotau concentrations). “Presymptomatic AD” applies to individuals who will develop AD and this can only be ascertained in families that are affected by rare autosomal dominant monogenic mutations (monogenic AD).

“Effective treatment regimen” refers to a treatment regimen that will offer beneficial response to a patient receiving the treatment.

An “effective amount” or “effective dose” of an agent refers to an amount or dose effective, for periods of time necessary, to achieve a desired result. For example, a “therapeutically effective amount” is an amount effective, for periods of time necessary, to treat an indicated disease, condition, clinical pathology, or symptom, e.g., to modify the course of progression of AD and/or to alleviate and/or prevent one or more symptoms of AD.

“Apolipoprotein E4 carrier” or “ApoE4 carrier,” used interchangeably herein with “apolipoprotein E4 positive” or “ApoE4 positive,” refers to an individual having at least one apolipoprotein E4 (or “ApoE4”) allele. An individual with zero ApoE4 alleles is referred to herein as being “ApoE4 negative” or an “ApoE4 non-carrier.” See also Prekumar, et al., 1996, Am. J Pathol. 148:2083-95.

The term “progression” as used herein refers to the worsening of a disease over time. The “progression rate” or “rate of progression” of a disease refers to how fast or slow a disease develops over time in a patient diagnosed with the disease. The progression rate of a disease can be represented by measurable changes over time of particular characteristics of the disease. A patient carrying particular genetic trait is said to have, or more likely to have, “increased progression rate” if the disease state progresses faster than those patients without such genetic trait. On the other hand, a patient responding to a therapy is said to have, or more likely to have, “decreased progression rate” if the disease progression slows down after the therapy, when compared to the disease state prior to the treatment or to other patients without the treatment.

The term “early Alzheimer's Disease” or “early AD” as used herein (e.g., a “patient diagnosed with early AD” or a “patient suffering from early AD”) includes patients with mild cognitive impairement, such as a memory deficit, due to AD and patients having AD biomarkers, for example amyloid positive patients.

The term “mild Alzheimer's Disease” or “mild AD” as used herein (e.g., a “patient diagnosed with mild AD”) refers to a stage of AD characterized by an Mini Mental State Examination (MMSE) score of 20 to 26.

The term “mild to moderate Alzheimer's Disease” or “mild to moderate AD” as used herein encompasses both mild and moderate AD, and is characterized by an MMSE score of 18 to 26.

The term “moderate Alzheimer's Disease” or “moderate AD” as used herein (e.g., a “patient diagnosed with moderate AD”) refers to a stage of AD characterized by an MMSE score of 18 to 19.

The term “MMSE” refers to the Mini Mental State Examination, which provides a score between 1 and 30. See Folstein, et al., 1975, J. Psychiatr. Res. 12:189-98. Scores of 26 and lower are generally considered to be indicative of a deficit. The lower the numerical score on the MMSE, the greater the tested patient's deficit or impairment relative to another individual with a lower score. An increase in MMSE score may be indicative of improvement in the patient's condition, whereas a decrease in MMSE score may denote worsening in the patient's condition.

The terms “pharmaceutical formulation” and “pharmaceutical composition” are used interchangeably herein and refer to a preparation which is in such form as to permit the biological activity of an active ingredient contained therein to be effective, and which contains no additional components which are unacceptably toxic to a subject to which the formulation would be administered.

In one embodiment, the present disclosure relates to a method of treating, or delaying the onset, or the progression of a disease characterized at least in part by beta amyloid expression, activity, or deposition in a subject, or for ameliorating at least one symptom associated with the disease, the method comprising administering a therapeutically effective amount of a first agent and a second agent to the subject. In some embodiments, the first agent is a myelin sheath protein and/or a synthetic polypeptide comprising a structure similar to said protein, and/or a homolog thereof, and/or a fragment thereof. In some embodiments, the second agent is an antibody, and/or an antibody fragment, and/or an antigen-binding fragment thereof that specifically binds beta amyloid, and/or amyloid-precursor-protein, and/or amyloid beta-derived diffusible ligands, and/or tau, and/or a fragment thereof, and/or any combination thereof. In some embodiments, the first agent and the second agent when administered to the subject act synergistically to reduce, and/or inhibit, and/or ameliorate at least one symptom and/or pathogenesis of the disease. In some embodiments, the second agent is an anti-tau antibody or an antibody that binds tau or a fragment thereof. In some embodiments, the second agent is an antibody that binds to beta amyloid and/or amyloid-precursor-protein, and/or amyloid beta-derived diffusible ligands, and/or a fragment thereof. In some embodiments, the second agent comprises at least one antibody or antigen-binding fragment thereof. In some embodiments, the second agent comprises two antibodies or antigen-binding fragment thereof. In some embodiments, the two antibodies are administered separately. In some embodiments, the two antibodies are administered at the same time. In some embodiments, the two antibodies are present in the same composition (e.g., same liquid composition). In some embodiments, the second agent comprises an anti-tau antibody and an antibody that binds beta amyloid and/or an antibody that binds amyloid-precursor-protein, and/or an antibody that binds amyloid beta-derived diffusible ligands. In some embodiments, the second agent comprises an anti-tau antibody and an antibody that binds beta amyloid. In some embodiments, the second agent is a bispecific antibody or a bispecific antibody fragment or antigen-binding fragment thereof. In some embodiments, the bispecific antibody or a bispecific antibody fragment or antigen-binding fragment thereof binds to tau and to beta amyloid and/or amyloid-precursor-protein, and/or amyloid beta-derived diffusible ligands.

In some embodiments the methods of the present disclosure provide for treating, inhibiting, or ameliorating a disease, or delaying the onset, or the progression of a disease characterized in part by beta amyloid expression, activity, or deposition in a subject in need thereof by administering a therapeutically effective amount of a myelin sheath protein and/or a synthetic polypeptide comprising a structure similar to said protein, and/or a homolog thereof, and/or a fragment thereof, as a first therapeutic agent and a therapeutically effective amount of one or more other therapeutic agents, wherein the disease or at least one symptom associated with the disease is lessened in severity or duration, or wherein the onset or progression of the disease or at least one symptom associated with the disease is delayed. In some embodiments, the one or more other therapeutic agents is at least one antibody or antigen-binding fragment thereof or at least one antibody fragment that specifically binds beta amyloid, and/or amyloid-precursor-protein, and/or amyloid beta-derived diffusible ligands, and/or tau, and/or a fragment thereof. In some embodiments, the one or more other therapeutic agents comprises an antibody or antigen-binding fragment thereof or an antibody fragment that specifically binds beta amyloid, and/or amyloid-pre cursor-protein, and/or amyloid beta-derived diffusible ligands, and an antibody or antigen-binding fragment thereof or an antibody fragment that specifically binds tau, and/or a fragment thereof. In some embodiments, the one or more other therapeutic agents is a bispecific antibody (e.g., bispecific to tau and beta amyloid). In some embodiments the at least one symptom associated with the disease is selected from the group consisting of memory loss, depression, anxiety, dementia, irritability, confusion, inattention, mood swings, and aggressive and/or apathetic behavior.

In one embodiment, the present disclosure relates to a method of treating or preventing a neurological disorder, including delaying the onset, slowing the progression or ameliorating symptoms of the disorder, comprising administering a therapeutically effective amount of at least one myelin sheath protein and/or a synthetic polypeptide comprising a structure similar to said protein, and/or a homolog thereof, and/or a fragment thereof, before, after, or concurrently with at least one antibody or antigen-binding fragment thereof or an antibody fragment that specifically binds beta amyloid, and/or amyloid-precursor-protein, and/or amyloid beta-derived diffusible ligand, and/or tau, and/or a fragment thereof, and/or any combination thereof, to a subject in need thereof. In some embodiments, the antibody or antigen-binding fragment thereof is bispecific. In some embodiments the bispecific antibody or antigen-binding fragment thereof binds beta amyloid, and/or amyloid-precursor-protein, and/or amyloid beta-derived diffusible ligand, and binds tau, and/or a fragment thereof. In some embodiments, the at least one antibody or antigen-binding fragment thereof or an antibody fragment comprises and anti-tau antibody and an antibody that binds beta amyloid, and/or amyloid-precursor-protein, and/or amyloid beta-derived diffusible ligand, and/or a fragment thereof.

In one embodiment, the method of the present disclosure relates to treating and/or preventing the progression of Alzheimer's disease and/or related tauopathies in a subject. In some embodiments, the method comprises administering to the subject a therapeutically effective amount of at least one myelin sheath protein and/or a synthetic polypeptide comprising a structure similar to said protein, and/or a homolog thereof, and/or a fragment thereof. In some embodiments, the method further comprises administering at least one additional agent. In some embodiments, the at least one additional agent is selected from a group consisting of or comprising an inhibitor of tau aggregation, an anti-amyloid antibody, an anti-amyloid aggregation inhibitor, an antibody that binds tau, an antibody that binds beta amyloid, an antibody that binds amyloid-precursor-protein, an antibody that binds amyloid beta-derived diffusible ligands, an antibody that binds a marker of Alzheimer's disease, and/or any combination thereof.

In one embodiment, the present disclosure provides a method for treating or lessening the severity of a disorder associated with amyloid-beta (1-42) peptide. In some embodiments, the present disclosure provides a method of modulating gamma-secretase in a patient. In some embodiments, the present disclosure provides a method of treating and/or lessening the severity of a neurodegenerative disorder in a patient. Such disorders include, but are not limited to, neurodegenerative disorders, such as Alzheimer's disease, Parkinson's disease, and Down's syndrome. In some embodiments, the present disclosure provides a method of treating and/or lessening the severity of a neurodegenerative disorder in the brain of a patient. In some embodiments, the present disclosure provides a method of treating and/or lessening the severity of Alzheimer's disease in the brain of a patient. In some embodiments, lessening the severity of the disease comprises administering a myelin sheath protein and/or a synthetic polypeptide comprising a structure similar to said protein, and/or a homolog thereof, and/or a fragment thereof, and an antibody or antigen-binding fragment thereof or an antibody fragment. In some embodiments, the antibody or antigen-binding fragment thereof or an antibody fragment binds to beta amyloid, and/or amyloid-precursor-protein, and/or amyloid beta-derived diffusible ligands, and/or a fragment thereof. In some embodiments, the antibody or antigen-binding fragment thereof or an antibody fragment binds to tau, and/or human tau, and/or a fragment thereof. In some embodiments, the antibody or antigen-binding fragment thereof or an antibody fragment comprises an antibody that binds tau (e.g., anti-tau antibody) and an antibody that binds beta amyloid, and/or amyloid-precursor-protein, and/or amyloid beta-derived diffusible ligands, and/or a fragment thereof. In some embodiments, the antibody or antigen-binding fragment thereof or an antibody fragment is bispecific. In some embodiments, the antibody or antigen-binding fragment thereof or an antibody fragment is bispecfic to at least one of beta amyloid, and/or amyloid-precursor-protein, and/or amyloid beta-derived diffusible ligands, and/or a fragment thereof, and to at least one of tau, and/or human tau, and/or a fragment thereof.

In one embodiment, the present disclosure relates to a method of monitoring the response of a subject being treated for neurological damage and/or Alzheimer's disease by administering neuroprotective agents, comprising the steps of: (a) determining the amount of at least one biomarker in a first biological sample taken from the subject prior to an initial treatment with a neuroprotective agent, wherein the neuroprotective agent comprises a combination of a myelin sheath protein and/or a synthetic polypeptide comprising a structure similar to said protein, and/or a homolog thereof, and/or a fragment thereof, and an antibody or antigen-binding fragment thereof or antibody fragment that specifically binds tau and/or beta amyloid, and/or amyloid-precursor-protein, and/or amyloid beta-derived diffusible ligands, and/or fragments thereof; (b) determining the amount of the biomarker in at least a second biological sample taken from the subject subsequent to the initial treatment with the neuroprotective agent; and (c) comparing the amount of the biomarker in the second biological sample with the amount of the biomarker in the first biological sample; such that a detectable reduction in the amount of the biomarker in the second biological sample compared to the amount of biomarker in the first biological sample indicates that the subject is responding positively to the treatment with the neuroprotective agent.

In some embodiments, a neurological damage is a condition or disease, or is caused by a condition, disease or event, selected from the group comprising cerebral ischemia, cerebral infarction, head trauma, contusion, spinal cord injury, subarachnoid hemorrhage, cerebral hemorrhage, aneurysmal hemorrhage, cardiac infarction, hypoxia, anoxia, surgery, Alzheimer's disease, Parkinson's disease, multiple sclerosis, HIV related neurodegeneration, cerebella degeneration, seizure and ataxia. In some embodiments, neurological damage includes any neurological damage caused by any condition, disease or event resulting in a partial or complete impairment in the supply of blood, oxygen or glucose to the CNS, or by a traumatic injury to the CNS. Non-limiting examples of such a condition, disease or event include cerebral ischemia, cerebral infarction, cerebral vasospasm, traumatic head injury, traumatic spinal cord injury, hemorrhage (such as subarachnoid hemorrhage, cerebral hemorrhage or aneurysmal hemorrhage), asphyxia (e. g., perinatal asphyxia), cardiac arrest, cardiac infarction, hypoxia or anoxia (e. g. , from drowning, suffocation, anesthesia administered during surgical procedures, pulmonary surgery, cardiac bypass, or use of a heart-lung machine), hypoglycemia, reperfusion injury, a progressive pathological condition (e. g., Alzheimer's Disease, Parkinson's Disease, Huntington's Disease, Multiple Sclerosis, HIV-related neurodegeneration or cerebella degeneration), seizure, glioblastoma, polyneuropathy, hydrocephalus, encephalitis, meningitis, epilepsy and schizophrenia, among others known in the art.

In some embodiments, the methods of the present disclosure are useful in circumstances where neurological damage to a mammalian subject, a human patient, has occurred, is in the process of occurring, or is anticipated to occur (e.g., prior to a surgical procedure), wherein the neurological damage can be monitored by a change in the amount of a particular measurable biomarker in a biological sample that can be obtained from the subject. Where there is an anticipation of potential neurological damage, e. g., that may result from an impending surgical procedure, the monitoring can be conducted to determine when the biomarker level has been reduced to a pre-determined acceptable level, or to a normal presurgical level. In some embodiments, the present disclosure is based on the identification of specific biomarkers of neurological damage, and the observation that efficacy of neuroprotective treatment can be monitored by tracking changes in the levels of one or more such biomarkers in response to such treatment. A biomarker useful according to the methods of the present disclosure can be a molecule that is present in undamaged tissues or cells and which, upon neurological injury or insult, is released from or secreted by the tissues or cells of the CNS into a biological tissue or fluid from which a biological sample can be obtained from a patient, such as, e. g., cerebrospinal fluid (CSF) or across the blood-brain barrier into non-CNS tissues such as the circulatory or lymph system, or into the saliva, or as excreted in the urine or in feces. In some embodiments, a biomarker can be a molecule produced de novo in response to neurological injury or insult, and which accumulates in a biological tissue or fluid from which a biological sample can be obtained from a patient, such as, e. g. , the CSF or non-CNS tissues such as the circulatory or lymph systems, or as excreted in the urine.

In some embodiments, a biomarker is any biological molecule that can be detected and quantified in a biological sample using standard biochemical assay methods, where the presence and/or quantity of the biomarker in the biological sample: (i) can be correlated with either the degree or ongoing progression of neurological damage; (ii) can be used to predict a patient's prognosis; (iii) can be used to select an appropriate neuroprotective treatment; and/or (iv) can be used to monitor the efficacy and progress of neuroprotective treatment.

In some embodiments, the biomarker is a member of the S-100 family of proteins. In some embodiments, the biomarker is S-100b (ββ) or S-100a (αβ) (see, Hermann et al., 2000, Stroke 31: 2670-2677; Hellmann et al., 2000, J. Neurotrauma 17: 113-122; U.S. Pat. No. 4,654,313 to Hartman, issued Mar. 31, 1987 ; and WO 00/52476). In some embodiments, the biomarker is neuron-specific enolase (NSE) (see, e. g., Herrmann et al., 2000, J. Neurotrauma 17: 113-122; and WO 00/52476). In some embodiments, the biomarker is glial fibrillary acidic protein (GFAP) (see, e. g., Hermann et al., 2000, Stroke 31: 2670-2677). In some embodiments, the biomarker is tau protein (see, e. g. , WO 99/45393 by the University of Cincinnati, published Sep. 10, 1999). In some embodiments, the biomarker is haptoglobin (see, e. g. , U.S. Pat. No. 5,429,947 to Merril et al., issued Jul. 4, 1995 ; U.S. Pat. No. 4,103,687 to Ishii, issued Aug. 1, 1978). In some embodiments, the biomarker is glutamate. Glutamate is a highly abundant excitatory neurotransmitter in the brain. Following severe head injury, local intercellular glutamate concentrations are believed to increase rapidly leading to excitatory cell death. The concentration of glutamate that appears in the CSF following severe head injury may reflect the extent of neuronal damage incurred. In some embodiments, the biomarker is a beta amyloid, and/or amyloid-precursor-protein, and/or amyloid beta-derived diffusible ligand. In some embodiments, the biomarker is creatine kinase (see, e. g. , U.S. Pat. No. 5,817,467 to Aoyama et al., issued Oct. 6, 1998). In some embodiments, the biomarker is F2-isoprostane (see, e. g. , U.S. Pat. No. 5,891,622 to Morrow et al., issued Apr. 6, 1999). In some embodiments, the biomarker is myelin basic protein (MBP) or thrombomodulin (see, e. g. , WO 00/52476). In some embodiments, the biomarker is a myelin sheath protein and/or a synthetic polypeptide comprising a structure similar to said protein, and/or a homolog thereof, and/or a fragment thereof.

In some embodiments, carrying out any of the methods of the present disclosure, comprises assaying the levels of either a single biomarker, or a panel of two or more different biomarkers, e. g. , both S-100b and NSE, or both tau and beta amyloid, or tau and beta amyloid and a myelin sheath protein. Assay of more than one biomarker may serve to increase the accuracy of determining the degree of neurological damage or of monitoring the response of the patient to neuroprotective treatment. Measurement of a plurality of biomarkers can be carried out by assaying the different biomarkers in either the same biological sample or in different biological samples taken from the same patient.

In some embodiments, the present disclosure provides a method of monitoring the response of a patient being treated for neurological damage by administration of a neuroprotective agent, comprising: (a) determining the amount of at least one biomarker in a first biological sample taken from the patient prior to an initial treatment with the neuroprotective agent; (b) determining the amount of the biomarker in at least a second biological sample from the patient subsequent to the initial treatment with the neuroprotective agent; and (c) comparing the amount of the biomarker present in the second biological sample with the amount of the biomarker present in the first biological sample; such that a detectable reduction, or prevention or slowing of an increase, in the amount of the biomarker present in the second biological sample, and/or in any subsequent biological samples, compared to the amount of biomarker present in the first biological sample indicates that the patient is responding positively to the treatment with the neuroprotective agent. Alternatively, where the amount of biomarker in a second or subsequent biological sample would tend to decrease naturally without treatment with a neuroprotective agent, e. g., as the response to the initial neurological damage subsides, a positive response to treatment with the neuroprotective agent can be indicated by a detectable increase in the rate of reduction of the amount of the biomarker present in the second or subsequent biological sample compared to the rate of reduction in the amount of biomarker that would be expected to occur without treatment with a neuroprotective agent (i. e., in a control biological sample) over a comparable time course.

In some embodiments, a lack of reduction (e. g. , either no detectable change or an increase in the total amount) in the amount of the biomarker in the second or subsequent biological sample compared to the amount of the biomarker in the first biological sample may indicate that the patient is not responding positively to the treatment with the neuroprotective agent.

In some embodiments, a lack of increase in the rate of reduction in the amount of the biomarker in the second or subsequent biological sample compared to the rate of reduction in the amount of biomarker that would be expected to occur in a control biological sample over a comparable time course may indicate that the patient is not responding positively to the treatment with the neuroprotective agent.

The amount of the biomarker (s) in a biological sample can be determined using those standard techniques currently known in the art or to be developed in the future. For example, each biomarker can be assayed using biomarker-specific antibodies and immunological methods known in the art. Any appropriate immunoassay method can be used, including radioimmunoassays, sandwich enzyme-linked immunoassays, competitive binding assays, homogeneous assays, and heterogeneous assays. Alternatively, the amount of biomarker (s) can be determined using other techniques such as magnetic resonance spectroscopy, HPLC or mass spectrometry. In any case, the assay method selected should be sensitive enough to be able to measure the particular biomarker in a concentration range from normal values found in healthy patients to elevated levels indicating neurological damage. The assay can be carried out in various formats including, e. g., in a microtiter plate format, using automated immunoassay analyzers known in the art.

In some embodiments, the methods of the present disclosure require that at least two biological samples are taken from the patient at different time points. In some embodiments, the first sample is obtained prior to an initial treatment with the neuroprotective agent, e. g., upon initial presentation at a physician's office or in a hospital emergency room setting as a result. In some embodiments, the second sample is obtained, and any subsequent samples are obtained, after neuroprotective treatment has begun. In some embodiments, the biomarker is monitored to determine: (i) if the amount of the biomarker is decreasing, (ii) if the rate of decrease in the amount of the biomarker is increasing, and/or (iii) if the amount of biomarker is stabilizing, any one of which may indicate that the patient is responding positively to the neuroprotective treatment depending upon the specific circumstances. If the biomarker level remains elevated over normal levels, or if the rate of decrease of the biomarker level is not sufficiently high, the neuroprotective treatment can be modified to a more aggressive protocol, such as by increasing the dosage or the number of treatments, and/or by changing the neuroprotective agent being administered to a more effective agent, and/or by combining the neuroprotective agent being used in the treatment with one or more other neuroprotective agents or therapies, or some combination thereof.

In some embodiments, the present disclosure further provides a method for treating a patient suffering from neurological damage by administering a neuroprotective agent, monitoring the level of at least one biomarker in a biological sample taken from the patient at one or more time points during treatment with the neuroprotective agent so as to determine whether an effective amount of the neuroprotective agent is being administered to the patient. An “effective amount of the neuroprotective agent” is being administered to the patient if the level of the biomarker in the biological sample detectably decreases, or if a previously observed rate of increase in the level of biomarker detectably slows, levels off, or is reversed, or if a previously observed rate of decrease in the level of the biomarker increases, in response to administration of the neuroprotective agent.

In some embodiments, the type of biological sample from which the amount of biomarker is determined will depend on a variety of factors such as the particular biomarker, where and when it is synthesized, where the biomarker may be stored in the tissues, and into what biological tissue or fluid it may be released or otherwise accumulate. In some embodiments, the biological sample will be selected from the group consisting of blood, a blood component such as serum or plasma, cerebrospinal fluid (CSF), saliva and urine. Where more than one biomarker is analyzed, the analysis can be conducted on the same or different biological samples obtained from the patient.

In some embodiments, a method of reducing production of amyloid beta, and/or of reducing plaque formation is disclosed. In some embodiments, the present disclosure provides methods of preventing or treating a disease associated with amyloid deposits of Abeta in the brain of a patient. In some embodiments, the method comprises administering to a subject a therapeutically effective amount of at least one myelin sheath protein and/or a synthetic polypeptide comprising a structure similar to said protein, and/or a homolog thereof, and/or a fragment thereof, in combination with at least one agent selected from an antibody or antigen-binding fragment thereof or an antibody fragment that specifically binds beta amyloid, and/or amyloid-precursor-protein, and/or amyloid beta-derived diffusible ligands, and/or tau, and/or a fragment thereof, and/or any combination thereof. In one embodiment, the present disclosure provides a method for reducing or inhibiting beta amyloid peptide production and amyloid plaque formation associated with Alzheimer's Disease comprising administering to a subject a therapeutically effective amount of at least one myelin sheath protein and/or a synthetic polypeptide comprising a structure similar to said protein, and/or a homolog thereof, and/or a fragment thereof, in combination with at least one agent selected from an antibody or antigen-binding fragment thereof or antibody fragment that specifically binds beta amyloid, and/or amyloid-precursor-protein, and/or amyloid beta-derived diffusible ligands, and/or tau, and/or a fragment thereof, and/or any combination thereof. In one embodiment, the present disclosure provides a method for reducing plaque formation in the brain of a subject, the method comprising administering to a subject a therapeutically effective amount of at least one myelin sheath protein and/or a synthetic polypeptide comprising a structure similar to said protein, and/or a homolog thereof, and/or a fragment thereof, in combination with at least one agent selected from an antibody or antigen-binding fragment thereof or antibody fragment that specifically binds beta amyloid, and/or amyloid-precursor-protein, and/or amyloid beta-derived diffusible ligands, and/or tau, and/or a fragment thereof, and/or any combination thereof. In some embodiments, the antibody or antigen-binding fragment thereof or antibody fragment comprises both an antibody or antigen-binding fragment thereof or antibody fragment that specifically binds beta amyloid, and/or amyloid-pre cursor-protein, and/or amyloid beta-derived diffusible ligands, and an antibody or antigen-binding fragment thereof or antibody fragment that specifically binds tau, and/or a fragment thereof. In some embodiments, the antibody or antigen-binding fragment thereof or antibody fragment is a bispecific antibody. In some embodiments, the bispecific antibody binds to at least one of beta amyloid, and/or amyloid-precursor-protein, and/or amyloid beta-derived diffusible ligands, and binds to tau, and/or a fragment thereof.

In some embodiments, any neurodegenerative condition associated with beta amyloid plaque formation or Abeta peptide production is a candidate for therapy that encompasses the compositions and methods disclosed herein. In addition, conditions that are known to overlap with Alzheimer's disease such as symptoms associated with Alzheimer's disease are present, for example, Lewy body dementia, can be targeted with the treatment methods provided by the present disclosure. In some embodiments, treatment of Alzheimer's disease according to the present disclosure can include amelioration of at least one symptom that may be caused by Alzheimer's disease. Examples include amelioration or suppression of cognitive impairment, amelioration or suppression of senile plaque formation, amelioration or suppression of synaptic dysfunction, and reduction or suppression of Abeta accumulation in brain tissues, blood, or such. In some embodiments, cognitive impairment can include, for example, memory impairment including long term/short term memory impairment, object recognition memory impairment, spatial memory impairment, and associative and emotional memory impairment. Lewy bodies refer to abnormal structures within nerve cells of the brain and it is estimated that up to 40 percent of people with Alzheimer's have Lewy bodies in the neocortex. Cerebral amyloid angiopathy (CAA) refers to the deposition of beta-amyloid in the media and adventitia of small and mid-sized arteries (and less frequently, veins) of the cerebral cortex and the leptomeninges. In some embodiments, it is a component of any disorder in which amyloid is deposited in the brain, and it is not associated with systemic amyloidosis.

In some embodiments, the method of the present disclosure comprises agents against cognitive impairment, Alzheimer's disease agents, agents for suppressing the progression of Alzheimer's disease, agents for suppressing senile plaque formation, agents for suppressing Abeta accumulation, anti-neurotoxic agents (agents for neutralizing neurotoxicity), agents for inhibiting Abeta amyloid fibril formation, and anti-synaptic toxicity agents (agents for neutralizing synaptic toxicity).

In one embodiment, the present disclosure relates to a method of treating a subject suffering from an amyloid disease and/or tauopathy comprising administering to the subject a therapeutically effective amount of at least one myelin sheath protein and/or a synthetic polypeptide comprising a structure similar to said protein, and/or a homolog thereof, and/or a fragment thereof, and at least one antibody or antigen-binding fragment thereof or antibody fragment that specifically binds tau, and/or a fragment thereof and/or at least one antibody or antigen-binding fragment thereof that specifically binds beta amyloid, and/or amyloid-precursor-protein, and/or amyloid beta-derived diffusible ligand, and/or a fragment thereof, and/or any combination thereof.

The present disclosure provides methods of treating an individual suffering from an amyloid disease by removing Abeta present in the individuals bloodstream or other bodily fluid. In some embodiments, agents associated with Abeta or fragments thereof, are administered to a patient suffering from or at risk for an amyloid disease. In some embodiments, an agent that binds to Abeta, can include an agent that binds Abeta 1-40 and/or Abeta 1-42. Non-limiting examples of an Abeta binding agent are apolipoprotein E, apolipoprotein J, serum amyloid P component, RNA aptamers directed against Abeta, alpha 1-antichymotrypsin, proteoglycans, gangliosides (such as monosiologanglioside GM1), vitronectin, vimentin, an antibody, and combinations thereof.

In one embodiment, the present disclosure relates to a method of improving cognitive impairment in a subject having beta amyloid deposits in brain tissue, the method comprising administering to the subject a therapeutically effective amount of at least one myelin sheath protein and/or a synthetic polypeptide comprising a structure similar to said protein, and/or a homolog thereof, and/or a fragment thereof, and at least one antibody or antigen-binding fragment thereof or antibody fragment that specifically binds beta amyloid, and/or amyloid-precursor-protein, and/or amyloid beta-derived diffusible ligand, and/or tau, and/or a fragment thereof, and/or any combination thereof, wherein the subject demonstrates an improvement in cognitive function without necessarily exhibiting a concurrent change in the beta amyloid plaque burden in the brain.

In some embodiments, the present disclosure provides for a method of improving cognitive impairment in a mammal having beta amyloid deposits in brain tissue, the method comprising administering a myelin sheath protein and/or a synthetic polypeptide comprising a structure similar to said protein, and/or a homolog thereof, and/or a fragment thereof as a first therapeutic agent, either alone, or in combination with one or more other therapeutic agents useful for treating the disease or at least one symptom of the disease. In some embodiments, the one or more other therapeutic agents useful for treating the disease or at least one symptom of the disease is an antibody. In some embodiments, the antibody or antigen binding fragment thereof or antibody fragment binds to beta amyloid, and/or amyloid-precursor-protein, and/or amyloid beta-derived diffusible ligand, and/or tau, and/or a fragment thereof, and/or any combination thereof. In some embodiments, the antibody or antigen binding fragment thereof or antibody fragment is a bispecific antibody. In some embodiments, the bispecific antibody binds beta amyloid, and/or amyloid-precursor-protein, and/or amyloid beta-derived diffusible ligand, and binds tau, and/or a fragment thereof. In some embodiments, the method provides for improvement of cognitive impairment in a subject having beta amyloid deposits in the brain, without necessarily altering the amount (increase or decrease) of beta amyloid in the brain. The improvement of cognitive impairment in a subject may be an improvement in learning performance, or an improvement in memory performance, or a decrease in memory loss, or a decrease in learning impairment. In some embodiments, the cognitive impairment is associated with Alzheimer's disease. In some embodiments, the treatment results in slowing the progression of any one or more cognitive or non-cognitive behavioral changes in the subject, including but not limited to memory loss, inability to learn, depression, anxiety, dementia, irritability, confusion, inattention, mood swings, diminished general locomotor and/or exploratory activity and aggressive and/or apathetic behavior. In some embodiments, subjects being treated may suffer from chronic neuroinflammation, which may contribute to the neurodegeneration and/or associated cognitive or non-cognitive dysfunction observed in patients with Alzheimer's disease, or any of the other neurodegenerative conditions. In some embodiments, subjects may show improved cognitive or non-cognitive behavioral symptoms following treatment, but will exhibit no change in the amount of beta-amyloid deposited in the brain. In some embodiments, the subjects being treated with the methods of the present disclosure will demonstrate a diminished immune response triggered by the amyloid plaque burden. The reduction in immune response may be shown by a reduction in the number, activated phenotype and/or the size of peri-plaque microglia.

In one embodiment, the present disclosure relates to a method of treating a subject having a brain Aβ disorder or predisposition to a brain Aβ disorder, comprising administering to the subject a therapeutically effective amount of at least one myelin sheath protein and/or a synthetic polypeptide comprising a structure similar to said protein, and/or a homolog thereof, and/or a fragment thereof, and at least one antibody or antigen-binding fragment thereof or antibody fragment that specifically binds tau, and/or a fragment thereof, and/or at least one antibody or antigen-binding fragment thereof or antibody fragment that specifically binds beta amyloid, and/or amyloid-precursor-protein, and/or amyloid beta-derived diffusible ligand, and/or a fragment thereof, and/or any combination thereof, thereby modulating production of AP in a tissue of the subject.

In some embodiments, the present disclosure provides a method, comprising the steps of assessing a subject for the presence of a brain Abeta disorder or predisposition to a brain Abeta disorder, and/or peripherally administering a therapeutically effective amount of at least one myelin sheath protein and/or a synthetic polypeptide comprising a structure similar to said protein, and/or a homolog thereof, and/or a fragment thereof, and at least one antibody or antigen-binding fragment thereof or antibody fragment that specifically binds tau, and/or a fragment thereof, and/or at least one antibody or antigen-binding fragment thereof or antibody fragment that specifically binds beta amyloid, and/or amyloid-precursor-protein, and/or amyloid beta-derived diffusible ligand, and/or a fragment thereof, and/or any combination thereof.

In some embodiments, the present disclosure provides a method of assessing risk of or presence of a brain Abeta disorder in a subject, comprising determining a level of Abeta in a peripheral tissue of said subject. In some other embodiments, the present disclosure provides a method of monitoring a brain Abeta disorder in a subject, comprising determining a level of Abeta in a peripheral tissue of said subject. In some embodiments, the peripheral tissue is blood, while in some embodiments, the peripheral tissue is serum. In some embodiments, monitoring comprises measuring Abeta in said peripheral tissue at a plurality of time points. In some embodiments of the methods of the present disclosure, the brain Abeta disorder is Alzheimer's disease. In some embodiments, the present disclosure provides methods of monitoring a brain Abeta disorder in a subject comprising analysis of expression or activity of a gene product in peripheral tissue of said subject. In some embodiments, the gene product is from a gene selected from the group consisting of Psen2, APP, Cib1, Ngrn, and Zfhx1b. In some embodiments, the present disclosure provides a method, comprising the steps of assessing a subject for the presence of a brain Abeta disorder or predisposition to a brain Abeta disorder, and peripherally administering an agent that inhibits the transport of peripheral Abeta across the blood brain barrier. In some embodiments, the agent is an anti-Abeta antibody. In some embodiments, the agent is an antibody that binds beta amyloid, and/or amyloid-precursor-protein, and/or amyloid beta-derived diffusible ligand, and/or a fragment thereof, and/or any combination thereof. In some embodiments, an antibody that binds beta amyloid (anti-Abeta antibody) and/or amyloid-precursor-protein, and/or amyloid beta-derived diffusible ligand, and/or a fragment thereof, and/or any combination thereof is administered with a myelin sheath protein and/or a synthetic polypeptide comprising a structure similar to said protein, and/or a homolog thereof, and/or a fragment thereof. In some embodiments, an antibody that binds beta amyloid (anti-Abeta antibody), and/or amyloid-precursor-protein, and/or amyloid beta-derived diffusible ligand, and/or a fragment thereof, and/or any combination thereof is administered with a myelin sheath protein and/or a synthetic polypeptide comprising a structure similar to said protein, and/or a homolog thereof, and/or a fragment thereof, and an antibody that binds tau and/or a fragment thereof. In some embodiments, an antibody that binds beta amyloid (anti-Abeta antibody), and/or amyloid-precursor-protein, and/or amyloid beta-derived diffusible ligand, and/or a fragment thereof, and/or any combination thereof also binds tau and/or a fragment thereof. In some embodiments, an antibody that binds beta amyloid (anti-Abeta antibody), and/or amyloid-precursor-protein, and/or amyloid beta-derived diffusible ligand, and/or a fragment thereof, and/or any combination thereof is administered with an antibody that binds tau and/or a fragment thereof. In some embodiments, the present disclosure provides a method of identifying a genetic target for treatment of a brain Abeta disorder, comprising comparing a liver gene expression profile of offspring from a first parent who has or who is predisposed to said Abeta disorder and a second parent having reduced susceptibility to said Abeta disorder, to identify a heritable genetic marker having a level of expression in liver, wherein increased or decreased expression of said heritable genetic marker in liver of said offspring relative to the level of expression in the liver of said first parent correlates with inheritance of said genetic marker from said second parent. In some embodiments, an AP related disorder include, but is not limited to, AD, brain trauma-related amyloid disease, Down syndrome, and inclusion body myositis.

In some embodiments, the present disclosure further comprises administering a compound selected from the group consisting STI-571, imatinib para-diaminomethylbenzene, N-desmethyl imatinib, LY450139, GSI-953, Flurizan, and E2012 compound, or a blood-brain barrier impermeable variant thereof, for use in the modulation of production of Abeta in peripheral tissue of a subject having or predisposed to developing a Abeta disorder. In some embodiments, the Abeta disorder is a brain Abeta disorder. In some embodiments, the partition coefficient is less than 2.0, or less than 1.5, and/or less than about 1.0. In some embodiments, the compound does not substantially cross the blood-brain barrier.

In some embodiments, the present disclosure relates to a method of delaying the onset of a neurological disorder (e.g., Alzheimer's Disease) and/or for treating or delaying the onset of amyloidosis-related diseases/disorders.

In some embodiments, the neurological disorder or the neurodegenerative disease is a disease associated with amyloidosis. In some embodiments, a disease associated with amyloidosis is AD, a condition that includes sporadic AD, ApoE4-related AD, other mutant APP forms of AD (e.g., mutations at APP717, which are the most common APP mutations), mutant PS1 forms of familial AD (FAD) (see, WO 96/34099), mutant PS2 forms of FAD (see, WO 97/27296), and alpha-2-macroglobulin-polymorphism-related AD. In some embodiments, the subject has Alzheimer's disease or a genetic predisposition for developing Alzheimer's disease. In some embodiments, the method of the present disclosure is directed to subjects that are at risk of developing Alzheimer's disease. In some embodiments, the subject has mild cognitive impairment, and/or at least one genotype known to be associated with Alzheimer's disease, and/or Trisomy 21, and/or subjects with surrogate markers indicating risk for Alzheimer's disease. In some embodiments, a subject that has at least one genotype known to be associated with Alzheimer's disease include subjects with ApoE4 genotype. In some embodiments, the disease can be the rare Swedish disease characterized by a double KM to NL mutation in amyloid precursor protein (APP) near the amino terminus of the beta AP portion of APP (Levy et al., Science 248:1124-26 (1990)). In some embodiments, the disease is hereditary cerebral hemorrhage with amyloidosis (HCHA or HCHWA)-Dutch type (Rozemuller et al., Am. J. Pathol. 142:1449-57 (1993); Roos et al., Ann. N.Y. Acad. Sci. 640:155-60 (1991); Timmers et al., Neurosci. Lett. 118:223-6 (1990); Haan et al., Arch. Neurol. 47:965-7 (1990)). In some embodiments, the disease can be sporadic cerebral amyloid angiopathy, hereditary cerebral amyloid angiopathy, Down syndrome, Parkinson-dementia of Guam, and age-related asymptomatic amyloid angiopathy (see, e.g., Haan and Roos, Clin. Neurol. Neurosurg. 92:305-310 (1990); Glenner and Murphy, N. Neurol. Sci. 94:1-28 (1989); Frangione, Ann. Med. 21:69-72 (1989); Haan et al., Clin. Neuro. Neurosurg 94:317-8 (1992); Fraser et al., Biochem. 31:10716-23 (1992); Coria et al., Lab. Invest. 58:454-8 (1988)). The actual amino acid composition and size of the beta AP (beta-amyloid peptide) involved in each of these diseases can vary (see e.g., Wisniewski et al., Biochem. Biophys. Res. Commun. 179:1247-54 (1991) and Biochem. Biophys. Res. Commun. 180:1528 (1991); Prelli et al., Biochem. Biophys. Res. Commun. 170:301-307 (1990); Levy, et al., Science 248:1124-26 (1990)).

In some embodiments, the Alzheimer's disease is an early-stage Alzheimer's disease. In some embodiments, an early-stage Alzheimer's disease includes prodromal Alzheimer's disease, and/or preclinical Alzheimer's disease, and/or mild cognitive impairment. Although mild cognitive impairment may be unrelated to Alzheimer's disease it is often a transitional stage to Alzheimer's disease or due to Alzheimer's disease. Preclinical and prodromal Alzheimer's disease may be asymptomatic stages, and are typically diagnosed by the presence of Alzheimer's disease related biomarkers. In some embodiments, the present disclosure provides a method of treating a disease selected from Alzheimer's disease (familial or sporadic), preclinical Alzheimer's disease, clinical Alzheimer's disease, prodromal Alzheimer's disease, mild cognitive impairment, Down's syndrome and cerebral amyloid angiopathy. In some embodiments, familial Alzheimer's disease is hereditary. In some embodiments, sporadic Alzheimer's disease is non-hereditary. In some embodiments, the Alzheimer's disease is a clinical disease. In some embodiments, an Aβ disorder is Alzheimer's disease.

In some embodiments, the methods of the present disclosure may be applied as a therapy for a subject having Alzheimer's disease and/or dementia and/or a neurological disorder, and/or the methods of the present disclosure may be applied as a prophylaxis against Alzheimer's disease and/or dementia and/or a neurological disorder for subject with such a predisposition, as in a subject, e.g., with a genomic mutation in the APP gene, with a genomic mutation in the ApoE gene, and/or with a genomic mutation in a presenilin gene. In some embodiments, the subject may have (or may be predisposed to developing or may be suspected of having) one or more of vascular dementia, or senile dementia, or Mild Cognitive Impairment. In some embodiments, in addition to Alzheimer's disease, the subject may have another amyloid-beta related disease such as cerebral amyloid angiopathy, or the subject may have amyloid deposits, especially amyloid-beta amyloid deposits in the subject's brain. In some embodiments, the subject may have familial, and/or sporadic, and/or idiopathic Alzheimer's disease, and/or cerebral amyloid angiopathy. In some embodiments, the subject has amyloid deposits. In some embodiments, the subject has amyloid-beta amyloid deposits. In some embodiments, the amyloid-beta amyloid deposits are present in the subject's brain.

In some embodiments, the methods of the present disclosure is directed to patients at risk for AD. The term “at risk for AD” is defined as patients being over the age of 60 and/or patients having a predisposition for AD. AD predisposing factors include (but are not limited to): (1) a genotype predisposing a patient to AD; (2) environmental factors predisposing a patient to AD; (3) past history of infection by viral and bacterial agents predisposing a patient to AD; and/or (4) vascular factors predisposing a patient to AD.

A number of genotypes are thought to predispose a patient to AD. These include, but are not limited to, the rare genotypes such as presenilin-1, presenilin-2, and amyloid precursor protein (APP) missense mutations which lead to some forms of familial early-onset AD, and the more common genotypes such as ApoE4 and alpha-2-macroglobulin genotypes, both of which are thought to increase the risk of acquiring sporadic late-onset AD. In addition, there is evidence that a family history of AD increases the risk of acquiring AD. Environmental factors have been proposed as predisposing a patient to AD, including exposure to aluminum. Prior infection by a number of viral and bacterial agents may predispose a patient to AD, including the herpes simplex virus and chlamydia pneumoniae. Other predisposing factors for AD can include risk factors for cardiovascular and/or cerebrovascular disease, including cigarette smoking, hypertension and diabetes. “At risk for AD” also encompasses any other predisposing factors not listed above or as yet identified and includes an increased risk for AD caused by head injury, medications, diet, or lifestyle.

In some embodiments, the present disclosure provides biomarkers, and/or methods of targeting biomarkers that are indicative of traumatic brain injury, neuronal damage, neural disorders, neurological disorder, brain damage, neural damage due to drug or alcohol addiction, Alzheimer's disease, amyloid related diseases, diseases associated with the brain or nervous system, such as the central nervous system. In some embodiments, the biomarkers are proteins, fragments or derivatives thereof, and are associated with neuronal cells, brain cells or any cell that is present in the brain and central nervous system. In some embodiments, the present disclosure provides methods of treating a subject suffering from a specific disease (e.g., Alzheimer's disease) with at least one biomarker and/or an antibody that binds to at least one biomarker.

In some embodiments the biomarkers are selected from at least one protein, peptide, variant or fragment thereof. For example, Axonal Proteins: alpha II spectrin (and SPDB)-1, NF-68 (NF-L)-2, Tau-3, alpha II, III spectrin, NF-200 (NF-H), NF-160 (NF-M), Amyloid precursor protein, alpha internexin; Dendritic Proteins: beta III-tubulin-1, p24 microtubule-associated protein-2, alpha-Tubulin (P02551), beta-Tubulin (P04691), MAP-2A/B-3, MAP-2C-3, Stathmin-4, Dynamin-1 (P21575), Phocein, Dynactin (Q13561), Vimentin (P31000), Dynamin, Profilin, Cofilin 1,2; Somal Proteins: UCH-L1 (Q00981)-1, Glycogen phosphorylase-BB-2, PEBP (P31044), NSE (P07323), CK-BB (P07335), Thy 1.1, Prion protein, Huntingtin, 14-3-3 proteins (e.g. 14-3-3-epsolon (P42655)), SM22-alpha, Calgranulin AB, alpha-Synuclein (P37377), beta-Synuclein (Q63754), HNP 22; Neural nuclear proteins: NeuN-1, S/G(2) nuclear autoantigen (SG2NA), Huntingtin; Presynaptic Proteins: Synaptophysin-1, Synaptotagmin (P21707), Synaptojanin-1 (Q62910). Synaptojanin-2, Synapsin1 (Synapsin-Ia), Synapsin2 (Q63537), Synapsin3, GAP43, Bassoon (NP_003449), Piccolo (aczonin) (NP_149015), Syntaxin, CRMP1, 2, Amphiphysin-1 (NP_001626), Amphiphysin-2 (NP_647477); Post-Synaptic Proteins: PSD95-1, NMDA-receptor (and all subtypes)-2, PSD93, AMPA-kainate receptor (all subtypes), mGluR (all subtypes), Calmodulin dependent protein kinase II (CAMPK)-alpha, beta, gamma, CaMPK-IV, SNAP-25, a-/b-SNAP; Myelin-Oligodendrocyte: Myelin basic protein (MBP) and fragments, Myelin proteolipid protein (PLP), Myelin Oligodendrocyte specific protein (MOSP), Myelin Oligodendrocyte glycoprotein (MOG), MOG35-55, MBP85-99, myelin associated protein (MAG), Oligodendrocyte NS-1 protein; Glial Protein Biomarkers: GFAP (P47819), Protein disulfide isomerase (PDI)-P04785, Neurocalcin delta, S100beta; Microglia protein Biomarkers: Iba1, OX-42, OX-8, OX-6, ED-1, PTPase (CD45), CD40, CD68, CD11b, Fractalkine (CX3CL1) and Fractalkine receptor (CX3CR1), 5-d-4 antigen; Schwann cell markers: Schwann cell myelin protein; Glia Scar: Tenascin; Hippocampus: Stathmin, Hippocalcin, SCG10; Cerebellum: Purkinje cell protein-2 (Pcp2), Calbindin D9K, Calbindin D28K (NP_114190), Cerebellar CaBP, spot 35; Cerebrocortex: Cortexin-1 (P60606), H-2Z1 gene product; Thalamus: CD15 (3-fucosyl-N-acetyl-lactosamine) epitope; Hypothalamus: Orexin receptors (OX-1R and OX-2R)-appetite, Orexins (hypothalamus-specific peptides); Corpus callosum: MBP, MOG, PLP, MAG; Spinal Cord: Schwann cell myelin protein; Striatum: Striatin, Rhes (Ras homolog enriched in striatum); Peripheral ganglia: Gadd45a; Peripherial nerve fiber (sensory+motor): Peripherin, Peripheral myelin protein 22 (AAH91499); Other Neuron-specific proteins: PH8 (S Serotonergic Dopaminergic, PEP-19, Neurocalcin (NC), a neuron-specific EF-hand Ca2+-binding protein, Encephalopsin, Striatin, SG2NA, Zinedin, Recoverin, Visinin; Neurotransmitter Receptors: NMDA receptor subunits (e.g. NR1A2B), Glutamate receptor subunits (AMPA, Kainate receptors (e.g. GluR1, GluR4), beta-adrenoceptor subtypes (e.g. beta(2)), Alpha-adrenoceptors subtypes (e.g. alpha(2c)), GABA receptors (e.g. GABA(B)), Metabotropic glutamate receptor (e.g. mGluR3), 5-HT serotonin receptors (e.g. 5-HT(3)), Dopamine receptors (e.g. D4), Muscarinic Ach receptors (e.g. M1), Nicotinic Acetylcholine Receptor (e.g. alpha-7); Neurotransmitter Transporters Norepinephrine Transporter (NET), Dopamine transporter (DAT), Serotonin transporter (SERT), Vesicular transporter proteins (VMAT1 and VMAT2), GABA transporter vesicular inhibitory amino acid transporter (VIAAT/VGAT), Glutamate Transporter (e.g. GLT1), Vesicular acetylcholine transporter, Vesicular Glutamate Transporter 1, [VGLUT1; BNPI] and VGLUT2, Choline transporter, (e.g. CHT1); Cholinergic Biomarkers: Acetylcholine Esterase, Choline acetyltransferase [ChAT]; Dopaminergic Biomarkers: Tyrosine Hydroxylase (TH), Phospho-TH, DARPP32; Noradrenergic Biomarkers: Dopamine beta-hydroxylase (DbH); Adrenergic Biomarkers: Phenylethanolamine N-methyltransferase (PNMT); Serotonergic Biomarkers Tryptophan Hydroxylase (TrH); Glutamatergic Biomarkers: Glutaminase, Glutamine synthetase; GABAergic Biomarkers: GABA transaminase [GABAT]), GABA-B-R2.

In some embodiments, the biomarker is selected from the group comprising S-100b, neuron-specific enolase (NSE), glial fibrillary acidic protein (GFAP), tau protein, haptoglobin, brain creatine kinase, isoprostane, myelin basic protein (MBP), myelin oligodendrocyte glycoprotein (MOG), beta amyloid, amyloid-precursor-protein, amyloid beta-derived diffusible ligands, thrombomodulin, or a fragment thereof, or any combination thereof.

Any animal that expresses the neural proteins, and/or a biomarker, and/or a peptide of the present disclosure, can be used as a subject. In some embodiments, the subject is a mammal, such as for example, a human, dog, cat, horse, cow, pig, sheep, goat, primate, rat, mouse and other vertebrates such as fish, birds and reptiles. In some embodiments, the subject is a human. In some embodiments, the subject is a subject suspected of having or at risk for developing traumatic or non-traumatic nervous system injuries, such as victims of brain injury caused by traumatic insults (e.g. gunshots wounds, automobile accidents, sports accidents, shaken baby syndrome), ischemic events (e.g. stroke, cerebral hemorrhage, cardiac arrest), spinal cord injury, neurodegenerative disorders (such as Alzheimer's, Huntington's, and Parkinson's diseases; Prion-related disease; other forms of dementia, and spinal cord degeneration), epilepsy, substance abuse (e.g., from amphetamines, methamphetamine/Speed, Ecstasy/MDMA, or ethanol and cocaine), and peripheral nervous system pathologies such as diabetic neuropathy, chemotherapy-induced neuropathy and neuropathic pain, peripheral nerve damage or atrophy (ALS), multiple sclerosis (MS).

In some embodiments, the expression of a biomarker indicative of a disease (e.g., indicative of Alzheimer's disease, and/or a neurological disease) is evaluated in the blood of the subject. In some embodiments, the expression of the biomarker is evaluated in PBMCs of the subject. In some embodiments, expression of the biomarker is evaluated at pretreatment. In some embodiments, expression of the biomarker is evaluated after beginning treatment with a myelin sheath peptide and/or a synthetic polypeptide comprising a structure similar to said protein, or a fragment thereof, and/or glatiramer acetate, and/or a copolymer 1 related peptide, and/or an antibody as disclosed in the present disclosure. In some embodiments, expression of the biomarker is evaluated 3 months after beginning treatment with a myelin sheath peptide and/or a synthetic polypeptide comprising a structure similar to said protein, or a fragment thereof, and/or glatiramer acetate, and/or a copolymer 1 related peptide, and/or an antibody as disclosed in the present disclosure. In some embodiments, expression of the biomarker is evaluated 12 months after beginning treatment with a myelin sheath peptide and/or a synthetic polypeptide comprising a structure similar to said protein, or a fragment thereof, and/or glatiramer acetate, and/or a copolymer 1 related peptide, and/or an antibody as disclosed in the present disclosure. In some embodiments, expression of the biomarker is evaluated 24 months after beginning treatment with a myelin sheath peptide and/or a synthetic polypeptide comprising a structure similar to said protein, or a fragment thereof, and/or glatiramer acetate, and/or a copolymer 1 related peptide, and/or an antibody as disclosed in the present disclosure. In some embodiments, if the human subject is identified as a myelin sheath peptide and/or a synthetic polypeptide comprising a structure similar to said protein, or a fragment thereof, and/or glatiramer acetate, and/or a copolymer 1 related peptide responder, the human subject is thereafter administered the pharmaceutical composition comprising a myelin sheath peptide and/or a synthetic polypeptide comprising a structure similar to said protein, or a fragment thereof, and/or glatiramer acetate, and/or a copolymer 1 related peptide and a pharmaceutically acceptable carrier in combination with at least one antibody of the present disclosure.

In some embodiments, the methods of the present disclosure related to treating or preventing the progression of Alzheimer's disease and/or a related tauopathy in a subject, or ameliorating at least one of the symptoms associated with Alzheimer's disease or a related tauopathy in a subject, comprises administering an effective amount of at least one antibody. In some embodiments, the effective dose of an antibody (e.g., of each antibody) is at least 1 mg/kg body weight of the subject, per dose. In some embodiments, the effective amount of each antibody is at least 10 mg/kg body weight of the subject, per dose. In some embodiments, at least one of the antibodies is administered in multiple dosages over a period of at least six months. In some embodiments, the method of the present disclosure relates to administering a myelin sheath protein and/or a synthetic polypeptide comprising a structure similar to said protein, or fragments thereof. In some embodiments, the method of the present disclosure further comprises administering an antibody. In some embodiments, the antibody is administered peripherally to a human subject to exert its beneficial effects. In some embodiments, the antibody is a bispecific antibody. In some embodiments, the bispecific antibody binds to tau and/or beta amyloid, and/or amyloid precursor protein, and/or amyloid beta derived diffusible ligands. In some embodiments, the antibody is a monoclonal antibody. In some embodiments, the antibody is not a bispecific antibody. In some embodiments, the antibody is a bispecific antibody. In some embodiments, the antibody, when administered peripherally to a human subject, binds to human amyloid protein, and/or beta amyloid, and/or soluble Abeta, and/or insoluble Abeta, and/or amyloid precursor protein, and/or amyloid beta derived diffusible ligands. In some embodiments, the antibody, when administered peripherally to a human subject, binds to tau, and/or soluble tau, and/or sarkosyl-insoluble tau, and/or to a fragment thereof. In some embodiments, the antibody, when administered peripherally to a human subject, binds to tau. In some embodiments, the antibody that is administered peripherally to a human subject, can bind to human amyloid protein, and/or beta amyloid, and/or solube Abeta, and/or insoluble Abeta, and/or amyloid precursor protein, and/or amyloid beta derived diffusible ligands, and can bind to tau, and/or soluble tau, and/or sarkosyl-insoluble tau (e.g., a bispecific antibody). In some embodiments, tau is in one or more pathological forms chosen from misordered tau, misdisordered tau, sarkosyl-insoluble tau, neurofibrillary tangles, neuropil threads, and neuritic plaques in a brain biopsy of a human Alzheimer's disease patient, and/or in a brain sample from an animal model of Alzheimer's disease. In some embodiments, the antibody, when administered peripherally to a human subject, exerts one or more effector-function mediated beneficial effects on the subject. In some embodiments, the antibody is delivered to the periphery by injection/implantation of an antibody-expressing cell into the subject's brain. In some embodiments, the antibody-expressing cell is a hybridoma cell.

In some embodiments, any of the methods of the present comprises at least two courses of therapy. In some embodiments, the course of therapy comprises in sequence a treatment session followed by an interval session of a non-treatment period. In some embodiments, a treatment session comprises administering at least one active agent. In some embodiments, an active agent can be a myelin sheath protein, a synthetic polypeptide comprising a structure similar to said protein, a homolog thereof, a fragment thereof, an antibody or antigen-binding fragment thereof that specifically binds beta amyloid, and/or amyloid-precursor-protein, and/or amyloid beta-derived diffusible ligands, and/or tau, a fragment thereof, or any combination thereof. In some embodiments, an active agent includes a myelin sheath protein, and/or a synthetic polypeptide comprising a structure similar to said protein, and/or a homolog thereof, and/or a fragment thereof, and an antibody or antigen-binding fragment thereof that specifically binds beta amyloid, and/or amyloid-precursor-protein, and/or amyloid beta-derived diffusible ligands, and an antibody or antigen-binding fragment thereof that specifically binds tau, a fragment thereof. In some embodiments, a treatment session comprises administering to a subject a therapeutically effective amount of at least one myelin sheath protein and/or a synthetic polypeptide comprising a structure similar to said protein, and/or a homolog thereof, and/or a fragment thereof, in combination with at least one agent selected from an antibody or antigen-binding fragment thereof that specifically binds beta amyloid, and/or amyloid-precursor-protein, and/or amyloid beta-derived diffusible ligands, and/or tau, and/or a fragment thereof, and/or any combination thereof. The length of the treatment and interval sessions may be determined by physicians in clinical trials directed to a certain patient population and then applied consistently to this patient population, without the need for monitoring the level of immunosuppression on a personal basis. In some embodiments, the treatment session comprises administering at least one active agent to a subject and the treatment session is maintained at least until the systemic presence of the at least one active agent reaches therapeutic levels, the administering is paused during the interval session, and the interval session is maintained as long as the level is above about 95%, 90%, 80%, 70%, 60% or 50% of said therapeutic level. In some embodiments, the treatment session may be between 3 days and four weeks long, for example between one and four weeks long. In some embodiments, the interval session (i.e., period of non-treatment) may be between one week and six months, for example between two weeks and six months long, or between 3 weeks and six months long. It should be understood that when an agent is administered during a treatment session at a relatively low frequency, for example once per week during a treatment session of one month, or once per month during a treatment session of six months, this treatment session is followed by a non-treatment interval session, the length of which is longer than the period between the repeated administrations during the treatment session (i.e. longer than one week or one month, respectively, in this example). The pause of one week or one month between the administrations during the treatment session in this example is not considered an interval session. The lengths of the treatment session and the interval session may be adjusted to the frequency of the administration such that, for example, a frequency of administering an active agent once every 3 days may result in a treatment session of 6 or 9 days and an interval session that is commenced accordingly. In some embodiments, a treatment session comprises administering at least one active agent daily, or once every two, three, four, five or six days, once weekly, once every two weeks, once every three weeks or once every four weeks. A treatment session of one active agent may be different than a treatment session of another active agent. For example, a treatment session of a myelin sheath protein, and/or a synthetic polypeptide comprising a structure similar to said protein, and/or a homolog thereof, and/or a fragment thereof may be the same, shorter, or longer than a treatment session of an antibody of the present disclosure (e.g., an antibody or antigen-binding fragment thereof that specifically binds beta amyloid, and/or amyloid-precursor-protein, and/or amyloid beta-derived diffusible ligands, and/or a fragment thereof, or an antibody or antigen-binding fragment thereof that specifically binds tau, and/or a fragment thereof) and vice versa. In some embodiments, the non-treatment session is longer than one week, and/or longer than one month. In some embodiments, the treatment session (e.g., treatment session of an active agent) is one day, two days, three days, four days, five days, six days, seven days, eight days, nine days, ten days, eleven days, twelve days, thirteen days, or fourteen days. In some embodiments, the treatment session is one week, two weeks, three weeks, four weeks, five weeks, six weeks, seven weeks, or eight weeks. In some embodiments, the treatment session is one month, two months, three months, four months, five months, six months, seven months, eight months, nine months, ten months, eleven months, or twelve months. In some embodiments, the treatment period is from one week to six months. In some embodiments, the treatment period is from two weeks to six months, or three weeks to six months, or four weeks to six months.

In some embodiments, any of the methods of the present disclosure may be intercalated with periods of non-treatment. In some embodiments, any of the methods of the present disclosure may be followed by an interval session of non-treatment. The term “non-treatment session” means a period of no treatment and refers to a session during which no active agent is administered to the individual being treated. In some embodiments, the non-treatment session is longer than one week, and/or longer than one month. In some embodiments, the non-treatment session is one day, two days, three days, four days, five days, six days, seven days, eight days, nine days, ten days, eleven days, twelve days, thirteen days, or fourteen days. In some embodiments, the non-treatment session is one week, two weeks, three weeks, four weeks, five weeks, six weeks, seven weeks, or eight weeks. In some embodiments, the non-treatment session is one month, two months, three months, four months, five months, six months, seven months, eight months, nine months, ten months, eleven months, or twelve months. In some embodiments, the non-treatment period is from one week to six months. In some embodiments, the non-treatment period is from two weeks to six months, or three weeks to six months, or four weeks to six months. In some embodiments, the non-treatment period is from about 3 days to four weeks. In some embodiments, the non-treatment session is longer than the period between repeated administrations during the treatment session. In some embodiments, the non-treatment session is shorter than the period between repeated administrations during the treatment session. In some embodiments, the non-treatment session improves efficiency of treatment.

### Antibodies

In some embodiments, an antibody or a binding fragment thereof comprises an immunoglobulin domain. In some embodiments, the antibody is a monoclonal antibody or a binding fragment thereof. In some embodiments, the antibody is a chimeric antibody or a fragment thereof. In some embodiments, the antibody or a binding fragment thereof is or comprises a human antibody, a humanized antibody, or a mouse antibody, or any combination thereof. In some embodiments, the monoclonal antibody or a binding fragment thereof is or comprises a human antibody, a humanized antibody, or a mouse antibody, or any combination thereof. In some embodiments, the antibody and/or monoclonal antibody and/or a binding fragment thereof inhibits formation of amyloid deposits in the subject. In some embodiments, an antibody or a binding fragment thereof is or comprises an IgG class antibody. In some embodiments, an antibody may include an immunoglobulin constant domain from any immunoglobulin, such as IgG-1, IgG-2, IgG-3, or IgG-4 subtypes, IgGM, IgA (including IgA-1 and IgA-2), IgE, IgD or IgM.

In some embodiments, the antibody of the present disclosure is used to treat an individual having an MMSE score of between 20 and 30, between 20 and 26, between 24 and 30, between 21 and 26, between 22 and 26, between 22 and 28, between 23 and 26, between 24 and 26, or between 25 and 26. In some embodiments, the patient has an MMSE score between 22 and 26. As used herein, an MMSE score between two numbers includes the numbers at each end of the range. For example, an MMSE score between 22 and 26 includes MMSE scores of 22 and 26.

In some embodiments, an antibody or antigen binding fragment thereof prevents the oligomerization of Abeta, particularly of Abeta40 and/or Abeta42 (e.g., through binding to its target). In some embodiments, the antibody has high affinity for oligomeric and/or monomeric forms of Abeta. In some embodiments, the antibody is capable of binding oligomeric and/or monomeric forms of beta amyloid. In some embodiments, the antibody recognizes/binds to amyloid precursor protein (APP). In some embodiments, the antibody does not substantially recognize amyloid precursor protein (APP). In some embodiments, the binding affinity to Abeta is a factor of at least two, or at least 5, or at least 10, or at least 50, or at least 100 times higher when compared to the binding affinity for APP.

In some embodiments, an antibody may be directed towards any region of Abeta or selected regions of amyloid precursor protein. In some embodiments, antibodies are directed to an epitope at the N-terminal region of Abeta, e.g., the epitope contains amino acids within 5 amino acids of the N-terminal amino acid. In some embodiments, the epitope lies within amino acids 3-8 of an Abeta peptide and the antibody targets corresponds to amino acids 1-17. In some embodiments, antibodies are directed at the mid-terminal region of Abeta, e.g., the epitope corresponds to amino acids 17-24 of human Abeta. In some embodiments, antibodies are directed to an epitope at the C-terminal region of Abeta, e.g., the epitope corresponds to amino acids 24-40/42/43 of human Abeta or contains amino acids within 5 amino acids of the C-terminal amino acid. In some embodiments, antibodies are directed to intracellular contained Abeta. In some embodiments, antibodies are directed towards the extraceullar fraction of Abeta. In some embodiments, an antibody may bind Abeta 40 and/or Abeta 42, selectively bind Abeta 42, bind all or some isoforms of Abeta, or the like. In some embodiments, an antibody binds intracellular Abeta. In some embodiments, an antibody binds an intracellular Abeta. In some embodiments, an antibody binds Abeta 40. In some embodiments, an antibody binds Abeta 42.

In some embodiments the antibody targets beta amyloid. In some embodiments the beta amyloid may comprise aggregated beta amyloid such as but not limited to soluble oligomers, insoluble fibrils deposited into amyloid plaque, or a combination thereof. In some embodiments, the antibody is Aducanumab (BIM037), Gantenerumab, Bapineuzumab, Crenezumab, Ponezumab, Solanezumab, SAR228810, MEDI1814, BAN2401, or any combination thereof. In some embodiments, the antibody can be an anti-ADDL (Amyloid-beta derived diffusible ligands) antibody as described in WO/2006/055178, bapineuzumab n-terminal (Elan &Wyeth), humanized m266 (LY2062430; Eli Lilly; e.g. as disclosed in U.S. Pat. No. 7,195,761) which binds epitopes 16-24 in the mid section of Aβ, RN-1219 (PF04360365; Pfizer Rinat) which binds c-terminal epitope of Aβ, fully humanized antibody R-1450 (Knappik et al, 2000; Hoffman-LaRoche/Morphosys), or the like. The antibody may include non-specific antibodies such as IVIg Gammaguard (Baxter), F(ab')2 fragments (Wilcock et al., 2003), non-antibody Aβ42 binding compounds (Matsuoka et al., 2005), or the like. In some embodiments, Aβ-derived diffusible ligands or ADDLs refer to soluble oligomers of amyloid β1-42 which are desirably composed of aggregates of less than eight or nine amyloid β1-42 peptides and are found associated with Alzheimer' s Disease.

In some embodiments, the antibody may target alpha-synuclein. In some embodiments, the antibody targeting alpha-synuclein is RG-7935, Posiphen, Affitope PD03A, Affitope PD01A, or any combination thereof.

In some embodiments, an antibody of the present disclosure binds a tau peptide. In some embodiments, an antibody or binding fragment thereof binds to human tau (pS422). In some embodiments, a tau peptide is extracellular tau (eTau).

In some embodiments, a tau peptide can comprise 1, 2, 3, 4, 5, 6, 7, 8, 9, 10, or more than 10, amino acid substitutions (e.g., conservative amino acid substitutions), relative to the amino acid sequence set forth in any one of SEQ ID NOs: 2-7. In some embodiments, the tau peptide comprises an amino acid sequence which comprises at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 2-7.

TABLE 1

| SEQ | |
|---|---|
| ID | |
| NO: | Sequence |
| 2 | AEPRQEFEVMEDHAGTYGLGDRKDQGGYTMHQDQEGDTDAGLK |
| | AEEAGIGDTPSLEDEAAGHVTQARMVSKSKDGTGSDDKKAKGA |
| | DGKTKIATPRGAAPPGQKGQANATRIPAKTPPAPKTPPSSGEP |
| | PKSGDRSGYSSPGSPGTPGSRSRTPSLPTPPTREPKKVAVVRT |
| | PPK |
| 3 | AEPRQEFEVMEDHAGTYGLGDRKDQGGYTMHQDQEGDTDAGLK |
| | AEEAGIGDTPSLEDEAAGHVTQARMVSKSKDGTGSDDKKAKGA |
| | DGKTKIATPRGAAPPGQKGQANATRIPAKTPPAPKTPPSSGEP |
| | PKSGDRSGYSSPGSPGTPGSRSRTPSLPTPPTREPKKVAVVR |
| 4 | AEPRQEFEVMEDHAGTYGLGDRKDQGGYTMHQDQEGDTDAGLK |
| | AEEAGIGDTPSLEDEAAGHVTQARMVSKSKDGTGSDDKKAKGA |
| | DGKTKIATPRGAAPPGQKGQANATRIPAKTPPAPKTPPSSGEP |
| | PKSGDRSGYSSPGSPGTPGSRSRTPSLPTPPTREPK |
| 5 | AEPRQEFEVMEDHAGTYGLGDRKDQGGYTMHQDQEGDTDAGLK |
| | AEEAGIGDTPSLEDEAAGHVTQARMVSKSKDGTGSDDKKAKGA |
| | DGKTKIATPRGAAPPGQKGQANATRIPAKTPPAPKTPPSSGEP |
| | PKSGDRSGYSSPGSPGTPGSR |
| 6 | AEPRQEFEVMEDHAGTYGLGDRKDQGGYTMHQDQEGDTDAGLK |
| | AEEAGIGDTPSLEDEAAGHVTQARMVSKSKDGTGSDDKKAKGA |
| | DGKTKIATPRGAAPPGQKGQANATRIPAKTPPAPK |
| 7 | AEPRQEFEVMEDHAGTYGLGDRKDQGGYTMHQDQEGDTDAGLK |
| | AEEAGIGDTPSLEDEAAGHVTQAR |
| 8 | MAEPRQEFEVMEDHAGTYGLGDRKDQGGYTMHQDQEGDTDAGL |
| | KAEEAGIGDTPSLEDEAAGHVTQARMVSKSKDGTGSDDKKAKG |
| | ADGKTKIATPRGAAPPGQKGQANATRIPAKTPPAPKTPPSSGE |
| | PPKSGDRSGYSSPGSPGTPGSRSRTPSLPTPPTREPKKVAVVR |
| | TPPKSPSS |

Administration of an anti-tau antibody to an individual can result in one or more of: reduction in the amount of free extracellular tau in brain tissue; reduction in the cell-to-cell spread (e.g., neuron-to-neuron spread) of Tau (e.g., Tau fragments); reduction in the amount of tau aggregates (e.g., intracellular (e.g., intraneuronal) tau aggregates); reduction in the amount of neurofibrillary tangles in brain tissue; reduction in the level of microglial activation and/or astrocyte activation; reduction in the amount of phosphorylated tau; reduction in the amount of hyperphosphorylated tau; reduction in total Tau (e.g., total intracellular Tau; and/or total extracellular Tau); reduction in free Tau (e.g., Tau that is not bound to a subject anti-tau antibody); reduction in neuronal hyperactivity; and reduction in the amount of N-terminal Tau fragments. “Total Tau” can include the sum total of full-length Tau of any isoform; and any N-terminal Tau fragments that are present and that display the epitope recognized by a subject anti-tau antibody. Reduction in phosphorylated Tau can be determined using any known method, e.g., an immunological method using an anti-phospho-tau antibody.

In some embodiments, administration of an anti-tau antibody, and/or anti-beta amyloid antibody, and/or anti-amyloid precursor protein antibody, and/or anti-amyloid beta derived diffusible ligands antibody, results in a change in one or more of: a) the amount of free extracellular tau in brain tissue; b) the amount of free extracellular tau in interstitial fluid (ISF); c) the amount of free extracellular tau in cerebrospinal fluid (CSF); d) the neuron-to-neuron spread of tau; e) the amount of intraneuron tau aggregates; f) the degree of microglial and/or astrocyte activation; g) the amount of phosphorylated or hyperphosphorylated tau; h) the amount of total Tau or free tau in ISF or CSF; i) the amount of intracellular N-terminal tau fragments; j) neuronal hyperactivity; k) the amount of Abeta 40 and/or Abeta 42 in CSF; 1) the Abeta plaque burden; m) secretion of Abeta 40 and/or Abeta 42 from a neuron; n) amyloid precursor protein (APP) promoter activity; o) APP mRNA and/or protein level; p) the activity of beta-secretase and/or gamma secretase; q) the activation state of an Abeta induced signaling pathway; r) the amount of intracellular total tau or free tau; s) the amount of anti-tau antibody-bound tau in ISF or CSF; and t) the amount of intracellular anti-tau antibody-bound tau.

Administration of an anti-tau antibody to an individual can result in one or more of: reduction in the amount of free extracellular tau peptide in brain tissue; reduction in the cell-to-cell spread (e.g., neuron-to-neuron spread) of tau peptides; reduction in the amount of tau aggregates (e.g., intracellular (e.g., intraneuronal) tau aggregates); and reduction in the amount of neurofibrillary tangles in brain tissue. Administration of an anti-tau antibody and/or anti-beta amyloid antibody, and/or anti-amyloid precursor protein antibody, and/or anti-amyloid beta derived diffusible ligands antibody, to an individual can in some cases improve cognitive function in the individual, or at least reduce the rate of decline of cognitive function in the individual.

In some embodiments, administration of an anti-tau antibody and/or anti-beta amyloid antibody, and/or anti-amyloid precursor protein antibody, and/or anti-amyloid beta derived diffusible ligands antibody to an individual reduces the amount of free extracellular tau peptide (e.g., the amount of free extracellular tau peptide in a brain tissue) by at least about 10%, at least about 20%, at least about 25%, at least about 50%, or more than 50%, compared to the amount of free extracellular tau peptide in the individual before administration with the anti-tau antibody.

In some embodiments, administration of an anti-tau antibody and/or anti-beta amyloid antibody, and/or anti-amyloid precursor protein antibody, and/or anti-amyloid beta derived diffusible ligands antibody, to an individual reduces the cell-to-cell (e.g., neuron-to-neuron) spread of a tau peptide (e.g., a pathological tau peptide) by at least about 10%, at least about 20%, at least about 25%, at least about 50%, or more than 50%, compared to the cell-to-cell spread before administration with an anti-tau antibody.

In some embodiments, administration of an anti-tau antibody and/or anti-beta amyloid antibody, and/or anti-amyloid precursor protein antibody, and/or anti-amyloid beta derived diffusible ligands antibody, to an individual reduces the amount of tau aggregates (e.g., intracellular (e.g., intraneuronal) tau aggregates) by at least about 10%, at least about 20%, at least about 25%, at least about 50%, or more than 50%, compared to the amount of tau aggregates before administration with the anti-tau antibody.

In some embodiments, administration of an anti-tau antibody and/or anti-beta amyloid antibody, and/or anti-amyloid precursor protein antibody, and/or anti-amyloid beta derived diffusible ligands antibody, to an individual reduces neurotoxicity in an individual; and/or reduces neuroinflammation in an individual; and/or reduces activation of astrocytes and microglia; and/or reduces induction of pathological electrophysiological effects; and/or reduces the amount of tau in exosomes.

In some embodiments, the present disclosure provides antibodies, e.g., isolated antibodies, specific for an eTau polypeptide. In some embodiments, an antibody of the present disclosure is also referred to as an “anti-tau antibody” or an “anti-eTau antibody.” In some embodiments, an anti-tau antibody of the present disclosure binds extracellular tau. “Extracellular tau” (“eTau”), as used herein, encompasses any Tau polypeptide that can be detected in cerebrospinal fluid (CSF) or interstitial fluid (ISF).

In some embodiments, an anti-tau antibody binds soluble Tau polypeptide (e.g., a soluble eTau polypeptide). In some embodiments, an anti-tau antibody binds soluble Tau polypeptide (e.g., a soluble eTau polypeptide); and/or does not substantially bind aggregated Tau polypeptide. In some embodiments, an anti-tau antibody binds a linear epitope on a soluble Tau polypeptide (e.g., a soluble eTau polypeptide of the present disclosure). In some cases, an anti-tau antibody binds a neoepitope; for example, in some cases, the epitope recognized and bound by an anti-tau antibody is not present in full-length Tau, but is generated upon cleavage of a Tau polypeptide to produce an extracellular Tau polypeptide. The neoepitope can be in a C-terminal region of the Tau polypeptide, or in an N-terminal region of the Tau polypeptide.

In some embodiments, the present disclosure provides an isolated antibody that specifically binds an epitope within an eTau polypeptide. In some embodiments, the present disclosure provides an isolated monoclonal antibody that specifically binds an epitope within an eTau polypeptide. In some embodiments, the present disclosure provides an isolated humanized monoclonal antibody that specifically binds an epitope within an eTau polypeptide.

In some embodiments, the present disclosure provides an isolated antibody that specifically binds an epitope within an eTau-1 polypeptide. An “eTau-1 polypeptide” includes, e.g., an eTau 2-166 polypeptide as depicted in SEQ ID NO: 4; an eTau 2-172 polypeptide as depicted in SEQ ID NO: 3; and an eTau 2-176 polypeptide as depicted in SEQ ID NO: 2. In some embodiments, the present disclosure provides an isolated monoclonal antibody that specifically binds an epitope within an eTau-1 polypeptide of the present disclosure. The present disclosure provides an isolated humanized monoclonal antibody that specifically binds an epitope within an eTau-1 polypeptide of the present disclosure. In some cases, an isolated antibody of the present disclosure binds an eTau-1 polypeptide; and does not bind an eTau-2 polypeptide (see SEQ ID NO: 5), an eTau-3 polypeptide (See SEQ ID NO: 6), or an eTau-4 polypeptide (See SEQ ID NO: 7). In some cases, an isolated antibody of the present disclosure binds an eTau-1 polypeptide, an eTau-2 polypeptide, an eTau-3 polypeptide, and an eTau-4 polypeptide. In some embodiments, the present disclosure provides an isolated antibody that specifically binds an epitope within an eTau-2 polypeptide. In some embodiments, the present disclosure provides an isolated antibody that specifically binds an epitope within an eTau-3 polypeptide. In some embodiments, the present disclosure provides an isolated antibody that specifically binds an epitope within an eTau-4 polypeptide.

In some embodiments, an antibody specific for a tau peptide specifically binds an acetylated form of the tau peptide and/or does not substantially bind an unacetylated form of the peptide. In other embodiments, an antibody specific for a tau peptide specifically binds an unacetylated form of the peptide.

In some embodiments, an anti-tau antibody can recognize a linear epitope in a tau peptide, or can recognize a conformational epitope. In some embodiments, the epitope recognized by an anti-tau antibody is a linear epitope.

In some embodiments, an anti-tau antibody is a monoclonal antibody. In some embodiments, an anti-tau antibody is an antibody fragment, e.g., an Fab, an Fab', an F(ab')2, a Fv fragment, a diabody, a linear antibody, a single-chain antibody, or other fragment capable of binding a tau peptide.

In some embodiments, an antibody of the present disclosure is an antibody that binds an epitope within amino acids 2-18 of human tau. In some embodiments, an antibody of the present disclosure is an antibody that binds an epitope within amino acids 15-24 of human tau. In some embodiments, an antibody of the present disclosure is an antibody that binds an epitope within amino acids 5-20 of human tau. In some embodiments, an antibody of the present disclosure is an antibody that binds an epitope within amino acids 19-46 of human tau. In some embodiments, an antibody of the present disclosure is an antibody that binds an epitope within amino acids 2 to 24 of human Tau. In some embodiments, human tau corresponds to SEQ ID NO: 8. In some embodiments, an antibody of the present disclosure binds Tau, binds monomeric Tau, oligomeric Tau, non-phosphorylated Tau, and/or phosphorylated Tau. In some embodiments, an antibody of the present disclosure binds to an epitope within amino acids 2 to 24 of human Tau (e.g., mature human tau). In some embodiments, an antibody of the present disclosure binds to an epitope within Tau amino acids 2 to 24 and/or binds monomeric Tau, and/or oligomeric Tau, and/or non-phosphorylated Tau, and/or phosphorylated Tau. In some embodiments, an antibody binds an epitope of human Tau having, or consisting of, the sequence AEPRQEFEVMEDHAGTYGLGDRK (SEQ ID NO: 9). In some embodiments, an antibody binds an epitope of cynomolgus monkey Tau having, or consisting of, the sequence AEPRQEFDVMEDHAGTYGLGDRK (SEQ ID NO: 10). In some embodiments, an antibody binds an epitope of human Tau having, or consisting of, the sequence AEPRQEFEVMEDHAGTYGLGDRK (SEQ ID NO: 11) and an epitope of cynomolgus monkey Tau having, or consisting of, the sequence AEPRQEFDVMEDHAGTYGLGDRK (SEQ ID NO: 12). In some embodiments, an antibody of the present disclosure binds to an epitope within amino acids 19 to 33, 19 to 42, 37 to 51, 100 to 114, 118 to 132, or 172 to 177 of human Tau (e.g., mature human tau). In some embodiments, an antibody of the present disclosure binds to an epitope within amino acids 19 to 33, 19 to 42, 37 to 51, 100 to 114, 118 to 132, or 172 to 177 of human Tau, and/or binds monomeric Tau, and/or oligomeric Tau, and/or non-phosphorylated Tau, and/or phosphorylated Tau.

In some embodiments, the antibody of the present disclosure comprises or consists of a Tau12 antibody (Ghoshal et al. (2002) Exp. Neurobiol. 177:475), a Tau13 antibody (Garcia-Sierra et al. (2003) J. Alz. Dis. 5:65), a TNT1 antibody (Kanaan et al. (2011) J. Neurosci. 31:9859), a 5A6 antibody (Johnson et al. (1997). J. Neurochemistry 68(1):430-433), a HJ9.4 antibody (Yanamandra et al. (2013) Neuron 80:402), an HT7 antibody, and/or a 6C10 antibody (US 2012/0183599). In some embodiments, an antibody or antigen binding fragment thereof that binds Tau is, or comprises, or consists of, AADVAC-1, AADVAC-2, ACI-35, BMS-986168, RG7345, TRx-237-015 (LMTX), AV-1451, AV-680, C2N-8E12, Posiphen, and/or any combination thereof.

In some embodiments, an anti-tau antibody for use as a medicament is provided. In some embodiments, an anti-tau antibody for use in treating a Tau protein associated disease or disorder is provided. In some embodiments, an anti-tau antibody for use in treating diseases or disorders caused by or associated with the formation of neurofibrillary tangles or neuropil threads is provided. In some embodiments, an anti-tau antibody for use in treating a tauopathy such as a neurodegenerative tauopathy is provided. Exemplary Tau protein associated diseases or disorders that can be treated with anti-tau antibodies include, without limitation, Alzheimer's Disease, amyotrophic lateral sclerosis, Parkinson's disease, Creutzfeldt-Jacob disease, Dementia pugilistica, Down's Syndrome, Gerstmann-Sträussler-Scheinker disease, inclusion-body myositis, prion protein cerebral amyloid angiopathy, traumatic brain injury, amyotrophic lateral sclerosis/parkinsonism-dementia complex of Guam, Non-Guamanian motor neuron disease with neurofibrillary tangles, argyrophilic grain dementia, corticobasal degeneration, diffuse neurofibrillary tangles with calcification, frontotetemporal dementia, frontotemporal dementia with parkinsonism linked to chromosome 17, Hallevorden-Spatz disease, multiple system atrophy, Niemann-Pick disease type C, Pallido-ponto-nigral degeneration, Pick's disease, progressive subcortical gliosis, progressive supranuclear palsy, Subacute sclerosing panencephalitis, Tangle only dementia, Postencephalitic Parkinsonism, and Myotonic dystrophy. In some embodiments, an anti-tau antibody for use in treating Alzheimer's Disease (AD) is provided herein. In some embodiments, an anti-tau antibody for use in treating moderate AD, mild to moderate AD, mild AD, early AD, or prodromal AD is provided herein. Further, Tau protein associated diseases or disorders that can be treated with an anti-tau antibody include diseases or disorders that are manifested in an impairment or loss of a cognitive function such as reasoning, situational judgment, memory capacity, learning, and/or special navigation. In some embodiments, an anti-tau antibody for use in a method of treatment is provided. In some embodiments, the present disclosure provides an anti-tau antibody for use in a method of treating an individual, having any one of the Tau associated diseases or disorders described herein, comprising administering to the individual an effective amount of the anti-tau antibody. In some embodiments, the method further comprises administering to the individual an effective amount of at least one additional therapeutic agent.

In some embodiments, an antibody or a binding fragment thereof may specifically bind an epitope containing the amino acid sequence DQGGYT (SEQ ID NO: 13). The epitope can be a linear or conformational epitopes and can be from about 6 to 22 amino acids in length. In some embodiments, the antibody binds to both monomeric tau and aggregated tau in human brain tissue from tauopathies. In some embodiments, the antibody is C₂N-8E12 (a humanized recombinant IgG4 anti-human tau antibody). The IgG4 backbone of C₂N-8E12 contains a S241P hinge stabilizing mutation that minimizes the formation of half-antibodies. C₂N-8E12 binds to amino acids 25-30 in human tau (DQGGYT (SEQ ID NO: 13)), a sequence that is present in all human tau splice variants as well as in amino-terminal fragments of tau. In some embodiments, an antibody (e.g., isolated humanized) or antigen-binding fragment specifically binds an epitope comprising the amino acid sequence DQGGYT (SEQ ID NO: 13). In some embodiments, the antibody or fragment may be a bispecific antibody or antigen-binding fragment where one arm of the antibody or fragment specifically binds an epitope comprising the amino acid sequence DQGGYT (SEQ ID NO: 13). In some embodiments, an antibody (e.g., isolated humanized) or antigen-binding fragment specifically binds an epitope comprising the amino acid sequence GYTMHQDQ (SEQ ID NO: 14). In some embodiments, a bispecific antibody or antigen-binding fragment is also provided where one arm of the antibody specifically binds an epitope comprising the amino acid sequence GYTMHQDQ (SEQ ID NO: 14). In some embodiments, an antibody or antigen-binding fragment thereof is a bispecific antibody or an antigen-binding fragment having two antigen-binding regions. In some embodiments, a bispecific antibody or antigen-binding fragment having two antigen-binding regions is provided. In some embodiments, one of the antigen-binding regions of the bispecific antibody or fragment thereof binds to tau, and/or human tau protein, and/or to human tau, and/or a fragment thereof. In some embodiments, one of the antigen-binding regions of the bispecific antibody or fragment thereof binds to human amyloid protein, and/or amyloid beta, and/or amyloid precursor protein, and/or amyloid beta derived diffusible ligands, and/or a fragment thereof. In some embodiments, the antibody is a bispecific antibody wherein the first antigen-binding region of the bispecific antibody or of the antigen-binding fragment having two antigen-binding regions binds to human beta amyloid, and/or amyloid-precursor-protein, and/or amyloid beta-derived diffusible ligands, and/or a fragment thereof, and the second antigen-binding region of the bispecific antibody or of the antigen-binding fragment having two antigen-binding regions binds to human tau, and/or tau, and/or a tau epitope, and/or a tau protein, and/or a fragment thereof. In some embodiments, the bispecific antibody or the antigen-binding fragment having two antigen-binding regions thereof comprises a first specificity towards: a) an epitope of beta-amyloid peptide that contains residues 28-35 of beta-amyloid, and/or b) an epitope of beta-amyloid peptide that contains residues 28-34 of beta-amyloid, and/or c) an epitope of beta-amyloid peptide that contains residues 28-33 of beta-amyloid, and/or d) an epitope within the region of residues 28-35 of beta-amyloid. In some embodiments, the bispecific antibody or the antigen-binding fragment having two antigen-binding regions thereof comprises a second specificity towards tau, and/or a tau protein, or a fragment thereof. In some embodiments, residues 24-34 of beta amyloid is Val Gly Ser Asn Lys Gly Ala Ile Ile Gly Leu (SEQ ID NO: 15), respectively. In some embodiments, residues 28-35 of beta amyloid is Lys Gly Ala Ile Ile Gly Leu Met (SEQ ID NO: 16), respectively.

In some embodiments, the present disclosure is directed to methods that relate to treating a disease with an antibody or antigen-binding fragment, wherein the antibody or fragment is administered in a dose to a subject having a disease. Suitable doses of the antibody or antigen-binding fragment may be expressed in terms of mg of drug per kg of subject's body weight. Suitable doses of the antibody or antigen-binding fragment include at least about 0.1 mg/kg, or about 0.2 mg/kg, or about 0.25 mg/kg, or about 0.3 mg/kg, or about 0.5 mg/kg, or about 0.75 mg/kg, or about 1 mg/kg, or about 1.25 mg/kg, or about 1.5 mg/kg, or about 2 mg/kg, or about 5 mg/kg, or about 7.5 mg/kg, or about 10 mg/kg, or about 12.5 mg/kg, or about 15 mg/kg, or about 20 mg/kg, or about 25 mg/kg, or about 30 mg/kg, or about 50 mg/kg, or about 100 mg/kg. Suitable doses of the antibody or antigen-binding fragment may be at most about 250 mg/kg, or at most about 200 mg/kg, or at most about 175 mg/kg, or at most about 150 mg/kg, or at most about 125 mg/kg, or at most about 100 mg/kg, or at most about 75 mg/kg, or at most about 50 mg/kg, or at most about 25 mg/kg, or at most about 20 mg/kg, or at most about 15 mg/kg. Any of the foregoing minima and maxima may be put together to define a range (for example, from about 0.1 mg/kg to about 250 mg/kg), so long as the minimum value of the range is lower than the maximum value of the range. Suitable doses of the antibody or antigen-binding fragment may be expressed in terms of mg of drug administered to a subject. Suitable doses of the humanized antibody or antigen-binding fragment include at least about 2.5 mg, or at least about 5 mg, or at least about 10 mg, or at least about 15 mg, or at least about 20 mg, or at least about 25 mg, or at least about 30 mg, or at least about 40 mg, or at least about 50 mg, or at least about 60 mg, or at least about 70 mg, or at least about 80 mg, or at least about 90 mg, or at least about 100 mg, or at least about 125 mg, or at least about 150 mg, or at least about 175 mg, or at least about 200 mg, or at least about 250 mg, or at least about 100 mg, or at least about 125 mg, or at least about 300 mg. Suitable doses of the antibody or antigen-binding fragment may be at most about 2500 mg, or at most about 2000 mg, or at most about 1500 mg, or at most about 1000 mg, or at most about 750 mg, or at most about 500 mg, or at most about 400 mg, or at most about 300 mg, or at most about 275 mg, or at most about 250 mg, or at most about 200 mg, or at most about 150 mg. Any of the foregoing minima and maxima may be put together to define a range (for example, from about 5 mg to about 2500 mg, so long as the minimum value of the range is lower than the maximum value of the range.

In some embodiments, an antibody of the present disclosure binds at least one marker associated with Alzheimer's disease, and/or at least one marker associated with an amyloidogenic pathology, and/or at least one marker associated with Parkinson's disease, and/or at least one marker associated with a neurological disease. In some embodiments, the marker is an antigen. In some embodiments, a marker and/or an antigen is derived from brain and/or neural tissue. Markers and/or antigens of interest (e.g., that are associated with Alzheimer's disease and/or amyloidogenic pathology, and/or neurological disease) include, but are not limited to P-S396-tau, P-T181-tau, Aβ, Aβ 1-42, Aβ 1-40, amyloid precursor protein (APP), amyloid precursor protein beta (APPβ), amyloid precursor protein alpha (APPα), soluble amyloid precursor protein beta (sAPPβ), soluble amyloid precursor protein alpha (sAPPα), and the like.

Other markers and/or antigens associated with amyloidogenic pathologies include, but are not limited to, islet amyloid protein (amylin) (IAPP), alpha-synuclein (SNCA), prion (PrP), huntingtin (HTT), calcitonin (Cal), atrial natriuretic factor (ANF), apolipoprotein A1 (ApoA1), serum amyloid a (AA), medin (Med), prolactin (Pro), transthyretin (TTR), lysozyme (Lys), beta 2 microglobulin (β2M), gelsolin (Gel), keratoepithelin (Ker), cystatin (Cys), immunoglobulin light chain AL (AL), S-IBM, cerebrovascular amyloid (CVA), and the like.

In some embodiments the marker and/or antigen of interest associated with Parkinson's disease is a-synuclein, although there are other antigens of interest in Parkinson's disease. Markers and/or antigens for multiple sclerosis include myelin basic protein (MBP). Markers and/or antigens for sleep disorders include orexin. Markers and/or antigens for glioma include epidermal growth factor receptor type III variant (EGFRvIII) and isocitrate dehydrogenase 1 (IDH1) Arg132 mutant. In some embodiments the markers and/or antigens of interest include but are not limited to Caveolin-1, EpCAM, FasL, TRAIL, Galectine3, CD151, Tetraspanin 8, EGFR, RPN2, CD44, CD47, CA15-3, CA27.29, CA19-9, CA-125, carcinoembryonic antigen (CEA), CD20, chromogranin A (CgA), cytokeratin fragments 21-1, HER4, HER2/neu, TGF-13, and the like.

### Myelin Sheath Protein

In some embodiments, the myelin sheath protein and/or the synthetic polypeptide comprising a structure similar to said protein, or a fragment thereof can be, for example, myelin oligodendrocyte glycoprotein (MOG), myelin basic protein (MBP), or proteolipid protein (PLP), myelin associated glycoprotein (MAG), peripheral myelin protein (PMP-22), P₀ protein, connexin 32 protein, Schwann cell myelin protein, oligodendrocyte-myelin glycoprotein (OMgp), glatiramer acetate, copolymer-1 related peptides, synthetic polypeptides thereof, or fragments thereof. In some embodiments, the synthetic polypeptide (e.g., a synthetic polypeptide comprising a structure similar to a myelin sheath protein) is at least about 80%, at least about 85%, at least about 90%, at least about 95%, at least about 97%, at least about 98%, or at least about 99% identical to a myelin sheath protein or a fragment thereof. In some embodiments, the myelin sheath protein and/or the synthetic polypeptide comprising a structure similar to said protein, or a fragment thereof may be in a form suitable for consumption by a subject, for example, a pill, tablet, caplet, soft and hard gelatin capsule, lozenge, sachet, cachet, vegicap, liquid drop, elixir, suspension, emulsion, solution, syrup, aerosol (as a solid or in a liquid medium) suppository, sterile injectable solution, and/or sterile packaged powder.

In some embodiments, a myelin sheath protein and/or a synthetic polypeptide comprising a structure similar to said protein, and/or a homolog thereof, and/or a fragment thereof is administered before, after, or concurrently with at least one antibody or antigen-binding fragment thereof of the present disclosure. In some embodiments, the myelin sheath protein and/or the synthetic polypeptide comprising a structure similar to said protein, or a fragment thereof can be administered before, after, or simultaneously with an antibody or antigen binding fragment thereof of the present disclosure. In some embodiments, the antibody or antigen binding fragment thereof is an antibody or antigen binding fragment thereof that binds tau and/or beta amyloid and/or amyloid precursor protein, and/or amyloid beta derived diffusible ligands, or fragments thereof.

In some embodiments, a myelin sheath protein and/or a synthetic polypeptide comprising a structure similar to said protein, and/or a fragment thereof is administered by peripheral administration, by intravenous administration, intraperitoneal administration, subcutaneous administration, intramuscular administration, oral administration, topical administration, transmucosal administration, or pulmonary administration. In some embodiments, a myelin sheath protein and/or a synthetic polypeptide comprising a structure similar to said protein, and/or a fragment thereof is administered by intravenous (bolus or infusion), intraperitoneal, subcutaneous, or intramuscular (e.g., injections). In some embodiments, a myelin sheath protein and/or a synthetic polypeptide comprising a structure similar to said protein, and/or a fragment thereof is administered by injection or by infusion. In some embodiments, amyelin sheath protein and/or a synthetic polypeptide comprising a structure similar to said protein, and/or a fragment thereof is administered orally. In some embodiments, the administration is by subcutaneous injection. In some embodiments, the administration is intramuscular. In some embodiments, the administration is intravenous. In some embodiments, the administration is by injection. In some embodiments, the administration is by infusion. In some embodiments, a myelin sheath protein and/or a synthetic polypeptide comprising a structure similar to said protein, and/or a fragment thereof (e.g., MOG, MBP, PLP, MAG, glatiramer acetate and/or a copolymer 1 related peptide) is administered orally. In some embodiments, a myelin sheath protein and/or a synthetic polypeptide comprising a structure similar to said protein, and/or a fragment thereof is administered by injection (e.g., subcutaneous injection). In some embodiments, a myelin sheath protein and/or a synthetic polypeptide comprising a structure similar to said protein, and/or a fragment thereof is administered by inhalation. In some embodiments, a myelin sheath protein and/or a synthetic polypeptide comprising a structure similar to said protein, and/or a fragment thereof can be provided in a tablet form, capsule, powder, aqueous, viscuous, solid, or liquid form.

In some embodiments, the composition of the present disclosure may be administered by any suitable mode of administration including orally, by inhalation, intramuscularly, subcutaneously or intradermally. Methods of administration and dosages will depend on the agent used and include, but are not limited to, parenteral, e.g., intravenous, intraperitoneal, intramuscular, subcutaneous, mucosal, e.g., oral, intranasal, buccal, vaginal, rectal, or intraocular, intrathecal, topical and intradermal routes, with or without adjuvant. Administration can be systemic or local.

In some embodiments, a myelin sheath protein and/or a synthetic polypeptide comprising a structure similar to said protein, and/or a fragment thereof may be administered one or more times per day (e.g., 1 or 2 times per day), once per week, once a month or continuously. In some embodiments, a subject may be treated less frequently, such as every other day or once a week, or more frequently. In some embodiments, a myelin sheath protein and/or a synthetic polypeptide comprising a structure similar to said protein, and/or a fragment thereof may be administered to a subject for about, or at least about, or at most about: 24 hours, 2 days, 3 days, 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, or 2 weeks to 24 months, periodically or continuously.

In some embodiments, a therapeutically effective amount of a myelin sheath protein and/or a synthetic polypeptide comprising a structure similar to said protein, and/or a fragment thereof to be administered to a subject is about, less than or equal to about, or more than or equal to about: 1 μg, 5 μg, 10 μg, 20 μg, 50 μg, 100 μg, 250 μg, 500 μg, 750 μg, 1 mg, 5 mg, 10 mg, 20 mg, 50 mg, 75 mg, 100 mg, 150 mg, 250 mg, 500 mg, 750 mg, 800 mg, 850 mg, 1000 mg, 1 kg, 5 kg, 10 kg, 20 kg, 50 kg, 75 kg, 100 kg, 150 kg, 250 kg, 500 kg, 750 kg, 800 kg, 850 kg, or 1000 kg.

In some embodiments, a therapeutically effective amount of a myelin sheath protein and/or a synthetic polypeptide comprising a structure similar to said protein, and/or a fragment thereof can range from about 1 mg/kg to about 50 mg/kg (e.g., from about 2.5 mg/kg to about 20 mg/kg or from about 2.5 mg/kg to about 15 mg/kg). Effective doses will also vary, as recognized by those skilled in the art, dependent on route of administration, excipient usage, and the possibility of co-usage with other therapeutic treatments including use of other therapeutic agents. For example, an effective dose of a myelin sheath protein and/or a synthetic polypeptide comprising a structure similar to said protein, and/or a fragment thereof to be administered to a subject, for example orally, can be from about 0.1 g to about 1 g, for example, from about 100 mg to about 800 mg (e.g., from about 120 mg to about 740 mg, from about 240 mg to about 720 mg; or from about 480 mg to about 720 mg; or about 720 mg). An effective dose of a myelin sheath protein and/or a synthetic polypeptide comprising a structure similar to said protein, and/or a fragment thereof can also be, for example, about 100 mg, about 200, mg, about 300 mg, about 400 mg, about 500 mg, about 600 mg, about 700 mg, about 800 mg, about 900 mg, or about 1 g. In some embodiments, the dosages may be administered one or more times per day. For example, 720 mg per day may be administered all at once or in separate administrations of 2, 3, 4, 5 or 6 (e.g., equal) doses.

In some embodiments, the effective dose may be per day, such as from about 0.1 g to about 1 g per day. In some embodiments, the effective dose may be per day administered, such as from about 0.1 g to about 1 g per day administered. In some embodiments, the effective dose may be per week, such as from about 0.1 g to about 1 g per week (or per week administered). In some embodiments, the effective dose may be per month, such as from about 0.1 g to about 1 g per month (or per month administered).

In some embodiments, a therapeutically effective amount of a myelin sheath protein and/or a synthetic polypeptide comprising a structure similar to said protein, and/or a fragment thereof can range from about 5 mg per day (and/or per day administered) to about 50 mg per day (and/or per day administered). In some embodiments, the therapeutically effective amount of a myelin sheath protein and/or a synthetic polypeptide comprising a structure similar to said protein, and/or a fragment thereof can range from about 5 mg per day (and/or per day administered) to about 30 mg per day (and/or per day administered), or from about 10 mg per day (and/or per day administered) to about 30 mg per day (and/or per day administered). In some embodiments, the therapeutically effective amount of a myelin sheath protein and/or a synthetic polypeptide comprising a structure similar to said protein, and/or a fragment thereof can range from about 15 mg per day (and/or per day administered) to about 25 mg per day (and/or per day administered). In other embodiments, the therapeutically effective amount of a myelin sheath protein and/or a synthetic polypeptide comprising a structure similar to said protein, and/or a fragment thereof is about 20 mg per day (and/or per day administered). In other embodiments, the therapeutically effective amount of a myelin sheath protein and/or a synthetic polypeptide comprising a structure similar to said protein, and/or a fragment thereof is less than the therapeutically effective amount when used in mono-therapy (e.g., 20 mg per day and/or per day administered). In other embodiments, the therapeutically effective amount of a myelin sheath protein and/or a synthetic polypeptide comprising a structure similar to said protein, and/or a fragment thereof is less than 20 mg per day (and/or per day administered). For example, the therapeutically effective amount of a myelin sheath protein and/or a synthetic polypeptide comprising a structure similar to said protein, and/or a fragment thereof can range from about 5 mg per day (and/or per day administered) to about 19 mg per day (and/or per day administered), or from about 10 mg per day (and/or per day administered) to about 19 mg per day (and/or per day administered) or from about 15 mg per day (and/or per day administered) to about 19 mg per day (and/or per day administered). Any of the above amounts of a myelin sheath protein and/or a synthetic polypeptide comprising a structure similar to said protein, and/or a fragment thereof can be administered all at once or in separate administrations of 2, 3, 4, 5 or 6 (e.g., equal) doses. In some embodiments, the administration is not of equal doses (e.g., the first administration may comprise a higher dose or a lower dose than the second or other administrations).

In some embodiments, the myelin sheath protein and/or a synthetic polypeptide comprising a structure similar to said protein, and/or a homolog thereof, and/or a fragment thereof, comprises, consists essentially of, or consists of Asp-Glu-Asn-Pro-Val-Val-His-Phe-Phe-Lys-Asn-Ile-Val-Thr-Pro-Arg-Thr (SEQ ID NO: 17). In some embodiments, the myelin sheath protein and/or a synthetic polypeptide comprising a structure similar to said protein, and/or a homolog thereof, and/or a fragment thereof, comprises, consists essentially of, or consists of Lys-Ser-His-Gly-Arg-Thr-Gln-Asp-Glu-Asn-Pro-Val-Val-His-Phe-Phe-Lys-Asn-Ile-Val-Thr (SEQ ID NO: 18). In some embodiments, the myelin sheath protein and/or a synthetic polypeptide comprising a structure similar to said protein, and/or a homolog thereof, and/or a fragment thereof, comprises, consists essentially of, or consists of Ala-Arg-Thr-Ala-His-Tyr-Gly-Ser-Leu-Pro-Gln-Lys-Ser-His-Gly (SEQ ID NO: 19). In some embodiments, the myelin sheath protein and/or a synthetic polypeptide comprising a structure similar to said protein, and/or a homolog thereof, and/or a fragment thereof, comprises, consists essentially of, or consists of His-His-Pro-Ala-Arg-Thr-Ala-His-Tyr-Gly-Ser-Leu-Pro-Gln-Lys (SEQ ID NO: 20). In some embodiments, the myelin sheath protein and/or a synthetic polypeptide comprising a structure similar to said protein, and/or a homolog thereof, and/or a fragment thereof, comprises, consists essentially of, or consists of Tyr-Gly-Ser-Leu-Pro-Gln-Lys-Ser-His-Gly-Arg-Thr-Gln-Asp-Glu (SEQ ID NO: 21). In some embodiments, the myelin sheath protein and/or a synthetic polypeptide comprising a structure similar to said protein, and/or a homolog thereof, and/or a fragment thereof, comprises, consists essentially of, or consists of Thr-Gln-Asp-Glu-Asn-Pro-Val-Val-His-Phe-Phe-Lys-Asn-Ile-Val-Thr-Pro-Arg (SEQID NO: 22). In some embodiments, the myelin sheath protein and/or a synthetic polypeptide comprising a structure similar to said protein, and/or a homolog thereof, and/or a fragment thereof, comprises, consists essentially of, or consists of Lys-Asn-Ile-Val-Thr-Pro-Arg-Thr-Pro-Pro-Pro-Ser-Gln-Gly-Lys-Gly (SEQ ID NO: 23). In some embodiments, the myelin sheath protein and/or a synthetic polypeptide comprising a structure similar to said protein, and/or a homolog thereof, and/or a fragment thereof, comprises, consists essentially of, or consists of Asn-Pro-Val-Val-His-Phe-Phe-Lys-Asn-Ile (SEQ ID NO: 24). In some embodiments, the myelin sheath protein and/or a synthetic polypeptide comprising a structure similar to said protein, and/or a homolog thereof, and/or a fragment thereof, comprises, consists essentially of, or consists of Pro-Val-Val-His-Phe-Phe-Lys-Asn-Ile-Val (SEQ ID NO: 25). In some embodiments, the myelin sheath protein and/or a synthetic polypeptide comprising a structure similar to said protein, and/or a homolog thereof, and/or a fragment thereof, comprises, consists essentially of, or consists of Val-Val-His-Phe-Phe-Lys-Asn-Ile-Val-Thr (SEQ ID NO: 26). In some embodiments, the myelin sheath protein and/or a synthetic polypeptide comprising a structure similar to said protein, and/or a homolog thereof, and/or a fragment thereof, comprises, consists essentially of, or consists of Val-His-Phe-Phe-Lys-Asn-Ile-Val-Thr-Pro (SEQ ID NO: 27). In some embodiments, the myelin sheath protein and/or a synthetic polypeptide comprising a structure similar to said protein, and/or a homolog thereof, and/or a fragment thereof, comprises, consists essentially of, or consists of Glu-Ala-Tyr-Lys-Ala-Ala-Glu-Lys-Ala-Tyr-Ala-Ala-Lys-Glu-Ala-Ala-Lys-Glu-Ala-Ala-Lys-Ala-Lys-Ala-Glu-Lys-Lys-Ala-Ala-Tyr-Ala-Lys-Ala-Lys-Ala-Ala-Lys-Tyr-Glu-Lys-Lys-Ala-Lys-Lys-Ala-Ala-Ala-Glu-Tyr-Lys-Lys-Lys (SEQ ID NO: 28). In some embodiments, a myelin sheath protein and/or a synthetic polypeptide comprising a structure similar to said protein, and/or a homolog thereof, and/or a fragment thereof, comprises an amino acid sequence which comprises at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 17-28.

In some embodiments, the myelin sheath protein and/or the synthetic polypeptide comprising a structure similar to said protein, and/or a fragment thereof comprises or consists of MOG 35-55, MOG 1-25, MOG 94-116, MOG 145-160, MOG 194-208, MBP 85-99, MBP 145-164, PLP 139-151, or PLP 95-116. In some embodiments, the myelin sheath protein and/or the synthetic polypeptide comprising a structure similar to said protein, and/or a fragment thereof, include or consist of MOG 35-55 (MEVGWYRPPFSRVVHLYRNGK; SEQ ID NO: 29 or MEVGWYRSPFSRVVHLYRNGK; SEQ ID NO: 30), MOG 1-25 (GQFRVIGPRHPIRALVGDEV; SEQ ID NO: 31), MOG 94-116 (GGFTCFFRDHSYQEEAAMELKVE; SEQ ID NO: 32), MOG 145-160 (VFLCLQYRLRGKLRAE; SEQ ID NO: 33), MOG 194-208 (LVALIICYNWLHRRL; SEQ ID NO: 34), MBP 85-99 (ENPVVHFFKNIVTPR; SEQ ID NO: 35), MBP 145-164 (VDAQGTLSKIFKLGGRDSRS; SEQ ID NO: 36), PLP 139-151 (CHCLGKWLGHPDKFVG; SEQ ID NO: 37), and PLP 95-116 (GAVRQIFGDYKTTICGKGLSAT; SEQ ID NO: 38). In some embodiments, a myelin sheath protein and/or a synthetic polypeptide comprising a structure similar to said protein, and/or a homolog thereof, and/or a fragment thereof, comprises an amino acid sequence which comprises at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID Nos 17-70, or to any sequence corresponding to the GenBank Accession Nos provided in this disclosure, or to glatiramer acetate, or to a copolymer-1 related peptide, or to any myelin sheath protein provided in this disclosure.

### Myelin Basic Protein (MBP):

A myelin protein which is a major constituent of the myelin sheath of oligodendrocytes and Schwann cells in the central and peripheral nervous system, respectively. Nucleic acid and protein sequences for MBP are publicly available. For example, GenBank Accession Nos. NM_001025081, NM_001025090, NM_001025092, and NM_002385 disclose exemplary human MBP nucleic acid sequences, and GenBank Accession Nos. NP_001020252, NP_001020261, NP_001020263, and NP_002376 disclose exemplary human MBP protein sequences, all of which are incorporated by reference herein. Similarly, GenBank Accession Nos. NM_001025251, NM_001025254, NM_001025255, NM_001025256, NM_001025258, and NM_001025259 disclose exemplary mouse MBP nucleic acid sequences, and GenBank Accession Nos. NP_001020422, NP_001020425, NP_001020426, NP_001020427, NP_001020429, and NP_001020430 disclose exemplary mouse MBP protein sequences, all of which are incorporated by reference herein. One of skill in the art can identify additional MBP sequences from human, mouse, or other species.

### Myelin Oligodendrocyte Glycoprotein (MOG):

A myelin protein which is a membrane protein expressed on the oligodendrocyte cell surface and the outermost surface of myelin sheaths. Nucleic acid and protein sequences for MOG are publicly available. For example, GenBank Accession Nos. NM_001008228, NM_001008229, NM_001170418, NM_002433, NM_206809, NM_206810, NM_206811, NM_206812, and NM_206814 disclose exemplary human MOG nucleic acid sequences, and GenBank Accession Nos. NP_001008229, NP_001008230, NP_001163889, NP_002424, NP_996532, NP_996533, NP_996534, NP_996535, and NP_996537 disclose exemplary human MOG protein sequences, all of which are incorporated by reference herein. Similarly, GenBank Accession No. NM_010814 discloses an exemplary mouse MOG nucleic acid sequence, and GenBank Accession No. NP_034944 discloses an exemplary mouse MOG protein sequence, both of which are incorporated by reference herein. One of skill in the art can identify additional MOG sequences from human, mouse, or other species.

### Proteolipid Protein (PLP):

A myelin protein which is the predominant myelin protein in the central nervous system. PLP is a transmembrane protein. Nucleic acid and protein sequences for PLP are publicly available. For example, GenBank Accession Nos. NM_000533, NM_001128834, and NM_199478 disclose exemplary human PLP nucleic acid sequences, and GenBank Accession Nos. NP_000524, NP_001122306, and NP_955772 disclose exemplary human PLP protein sequences, all of which are incorporated by reference herein. Similarly, GenBank Accession No. NM_011123 discloses an exemplary mouse PLP nucleic acid sequence, and GenBank Accession No. NP_035253 discloses an exemplary mouse PLP protein sequence, both of which are incorporated by reference herein. One of skill in the art can identify additional PLP sequences from human, mouse, or other species.

### Glatiramer Acetate:

As used herein, the terms “Copolymer 1”, “Cop 1”, “Cop-1”, “Copaxone”, “glatiramer acetate” and “GA” are each used interchangeably. Glatiramer acetate (or glatiramer, or GA) is marketed under the tradename Copaxone (Registered trademark). GA is typically administered at a daily dose of about 1 to about 60 mg. Glatiramer acetate is a mixture of polypeptides composed of alanine, glutamic acid, lysine, and tyrosine in a molar ratio of approximately 4.6:1.5:3.6:1.0, respectively, which is synthesized by chemically polymerizing the four amino acids, forming products with average molecular weights ranging from about 4,000 to about 13,000 daltons. The corresponding molar fractions are approximately 0.427 for alanine, 0.141 for glutamic acid, 0.337 for lysine and 0.093 for tyrosine, and may vary by about +/−10%. A method for synthesis of glatiramer acetate has been described for example in U.S. Pat. No. 7,049,399. In some embodiments, glatiramer acetate and/or a copolymer 1 related peptide is administrated at a daily dose of 20 mg injected subcutaneously (SC). In some embodiments, the therapeutically effective dose of glatiramer acetate and/or a copolymer 1 related peptide is 40 mg/ml. In some embodiments, glatiramer acetate and/or a copolymer 1 related peptide is administered at a dose of 40 mg/ml. In some embodiments, 40 mg glatiramer acetate and/or a copolymer 1 related peptide is administered to a subject. In some embodiments, a composition comprising glatiramer acetate and/or a copolymer 1 related peptides comprises mannitol.

The dosage of the active ingredient to be administered may be determined by the physician, according to the age of the patient and stage of the disease. For example, for Cop 1 and/or copolymer 1 related peptide, the dosage may be chosen from a range of 1-80 mg, for example 20 mg, although any other suitable dosage is encompassed by the present disclosure. The treatment can be carried out by administration of repeated doses at suitable time intervals, according to the stage of the disease, the age and condition of the patient. In some embodiments, Cop 1 and/or copolymer 1 related peptide may be administered daily. In some embodiments, the administration may be made according to a regimen suitable for immunization, for example, at least once a week, once a month or at least once every 2 or 3 months, or less frequently, but any other suitable interval between the immunizations is envisaged by the present disclosure according to the condition of the patient.

In some embodiments, a subject is administered three injections of GA and/or a copolymer 1 related peptide for every seven days with at least one day between each injection. In some embodiments, possible injection schedules include, but is not limited to, Day 1, Day 3, Day 5; Day 1, Day 3, Day 6; Day 1, Day 3, Day 7; Day 1, Day 4, Day 6; Day 1, Day 4, Day 7; Day 1, Day 5, Day 7; Day 2, Day 4, Day 6; Day 2, Day 4, Day 7; Day 2, Day 5, Day 7; or Day 3, Day 5, Day 7. In some embodiments, 40 mg GA and/or a copolymer 1 related peptide is administered three times a week. In some embodiments, 40 mg GA and/or a copolymer 1 related peptide is administered every other day. In some embodiments, 20 mg GA and/or a copolymer 1 related peptide is administered every day. In some embodiments, 20 mg GA and/or a copolymer 1 related peptide is administered at least once a day. In some embodiments, 20 mg GA and/or a copolymer 1 related peptide is administered at least once every 24 hours.

In some embodiments, glatiramer acetate and/or a copolymer 1 related peptide is administered at about, at least about, or at most about: 5 ng, 10 ng, 100 ng, 500 ng, 750 ng, 1 microgram, 5 micrograms, 10 micrograms, 50 micrograms, 100 micrograms, 250 micrograms, 500 micrograms, 750 micrograms, 1 mg, 5 mg, 7 mg, 9 mg, 10 mg, 12 mg, 15 mg, 17 mg, 20 mg, 25 mg, 30 mg, 35 mg, 40 mg, 45 mg, 50 mg, 60 mg, 70 mg, 80 mg, 90 mg, and/or 100 mg.

In some embodiments, glatiramer acetate and/or a copolymer 1 related peptide is administered to a subject at a range of about 10 mg to about 50 mg, of about 20 mg to about 40 mg, of about 750 micrograms to about 40 mg, of about 5 ng to about 100 mg, of about 5 mg to about 45 mg, or of about 5 microgram to about 20 mg.

In some embodiments, glatiramer acetate and/or a copolymer 1 related peptide is administered once a day, twice a day, three times a day, every day, every other day, once a week, twice a week, three times a week, four times a week, five times a week, six times a week, seven times a week, once a month, twice a month, three times a month, four times a month, five times a month, six times a month, seven times a month, eight times a month, nine times a month, ten times a month, fifteen times a month, twenty times a month, twenty-five times a month, thirty times a month, more than thirty times a month, once a year, twice a year, three times a year, four times a year, five times a year, six times a year, seven times a year, eight times a year, nine times a year, ten times a year, fifteen times a year, twenty times a year, twenty-five times a year, thirty times a year, forty times a year, fifty times a year, 75 times a year, 80 times a year, 90 times a year, 100 times a year, 120 times a year, and/or more than 120 times a year.

In some embodiments, the pharmaceutical composition comprising glatiramer acetate and/or a copolymer 1 related peptide is a unit dose of a 1.0 ml aqueous solution comprising 40 mg of glatiramer acetate. In some embodiments, the pharmaceutical composition comprising glatiramer acetate and/or a copolymer 1 related peptide is a unit dose of a 1.0 ml aqueous solution comprising 20 mg of glatiramer acetate. In some embodiments, the pharmaceutical composition comprising glatiramer acetate and/or a copolymer 1 related peptide is a unit dose of a 0.5 ml aqueous solution comprising 20 mg of glatiramer acetate. In some embodiments, the pharmaceutical composition comprising glatiramer acetate and/or a copolymer 1 related peptide is in a prefilled syringe for self administration by the human subject. In some embodiments, the human subject is a naive patient. In some embodiments, the human subject has previously been administered glatiramer acetate.

In some embodiments, the Cop 1 or a Copolymer 1 related peptide or polypeptide is represented by a random copolymer consisting of a suitable ratio of a positively charged amino acid such as lysine or arginine, in combination with a negatively charged amino acid such as glutamic acid or aspartic acid, optionally in combination with a non-charged neutral amino acid such as alanine or glycine, serving as a filler, and optionally with an amino acid adapted to confer on the copolymer immunogenic properties, such as an aromatic amino acid like tyrosine or tryptophan. Such copolymers are disclosed, for example, in WO 00/05250, the entire contents of which are herewith incorporated herein by reference.

In some embodiments, the Copolymer 1 or a Copolymer 1 related peptide or polypeptide is a copolymer selected from the group consisting of random copolymers comprising one amino acid selected from each of at least three of the following groups: (a) lysine and arginine; (b) glutamic acid and aspartic acid; (c) alanine and glycine; and (d) tyrosine and tryptophan. The amino acids may be L- or D-amino acids or mixtures thereof The present disclosure contemplates the use of copolymers containing both D- and L-amino acids, as well as copolymers consisting essentially of either L- or D-amino acids. In some embodiments, the copolymer and/or a Copolymer 1 related peptide or polypeptide contains four different amino acids, each from a different one of the groups (a) to (d).

In some embodiments, the copolymer and/or a Copolymer 1 related peptide or polypeptide is composed of a mixture of random polypeptides consisting essentially of the amino acids L-glutamic acid (E), L-alanine (A), L-tyrosine (Y) and L-lysine (K) in an approximate ratio of 1.5:4.8:1:3.6, having a net overall positive electrical charge and of a molecular weight from about 2 KDa to about 40 KDa. In some embodiments, the Cop 1 and/or a Copolymer 1 related peptide or polypeptide has average molecular weight of about 2 KDa to about 20 KDa, or from about 4.7 KDa to about 13 K Da, or from about 4 KDa to about 8.6 KDa, or from about 5 KDa to 9 KDa, or from about 6.25 KDa to 8.4 KDa. In some embodiments, the Cop 1 and/or a Copolymer 1 related peptide or polypeptide has average molecular weight of about 13 KDa to about 20 KDa, or from about 13.5 KDa to about 18 KDa, with an average of about 15 KDa to about 16 KD, or of 16 kDa. Other average molecular weights for Cop 1, and/or for a Copolymer 1 related peptide or polypeptide lower than 40 KDa, are also encompassed by the present disclosure. Copolymer 1 of said molecular weight ranges can be prepared by methods known in the art, for example by the processes described in U.S. Pat. No. 5,800,808. The Copolymer 1 and/or a Copolymer 1 related peptide or polypeptide may be a polypeptide comprising from about 15 to about 100, or from about 40 to about 80 amino acids in length.

In some embodiments, a copolymer 1 related peptide is a random copolymer of 4 amino acids in which one or more of the following substitutions is made: aspartic acid for glutamic acid, glycine for alanine, arginine for lysine, and tryptophan for tyrosine, which is expected to have the same activity of Copolymer. In some embodiments, the Copolymer 1 related peptide or polypeptide is a copolymer of three different amino acids each from a different one of three groups of the groups (a) to (d) (e.g., (a) lysine and arginine; (b) glutamic acid and aspartic acid; (c) alanine and glycine; and (d) tyrosine and tryptophan). These copolymers may be referred to as terpolymers. In some embodiments, the terpolymer contains tyrosine (Y), alanine (A), and lysine (K), hereinafter designated YAK, in which the average molar fraction of the amino acids can vary: Y, A and K can be present in a mole fraction of about 0.05-0.250, 0.3-0.6; and 0.1-0.5, respectively, or the molar ratios of Y, A and K are about 0.10:0.54:0.35, respectively. In some embodiments, it is possible to substitute arginine for lysine, glycine for alanine, and/or tryptophan for tyrosine. In some embodiments, the terpolymer contains tyrosine (Y), glutamic acid (E), and lysine (K), which the average molar fraction of the amino acids can vary: E, Y and K can be present in a mole fraction of about 0.005-0.300, 0.005-0.250, and 0.3-0.7, respectively. In some embodiments, the molar ratios of E, Y and K are about 0.26:0.16:0.58, respectively. It is possible to substitute aspartic acid for glutamic acid, arginine for lysine, and/or tryptophan for tyrosine. In some embodiments, the terpolymer contains lysine (K), glutamic acid (E), and alanine (A), hereinafter designated KEA, in which the average molar fraction of the amino acids can vary: E, A and K can be present in a mole fraction of about 0.005-0.300, 0.005-0.600, and 0.2-0.7, respectively. In some embodiments, the molar ratios of E, A and K are about 0.15:0.48:0.36, respectively. It is possible to substitute aspartic acid for glutamic acid, glycine for alanine, and/or arginine for lysine. In some embodiments, the terpolymer contains tyrosine (Y), glutamic acid (E), and alanine (A), which the average molar fraction of the amino acids can vary: Y, E and A can be present in a mole fraction of about 0.005-0.250, 0.005-0.300, and 0.005-0.800, respectively. In some embodiments, the molar ratios of E, A, and Y are about 0.21:0.65:0.14, respectively. It is possible to substitute tryptophan for tyrosine, aspartic acid for glutamic acid, and/or glycine for alanine. The average molecular weight of the terpolymers YAK, YEK, KEA and YEA can vary between about 2 KDa to 40 KDa, or between about 3 KDa to 35 KDa, or between about 5 KDa to 25 KDa.

In some embodiments, a copolymer 1 related peptide has the sequence of any of SEQ ID NOs 39-70 of Table 2 below. In some embodiments, the copolymer 1 related peptide comprises an amino acid sequence which comprises at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 39-70.

TABLE 2

| SEQ ID NO: | Sequence |
|---|---|
| 39 | AAAYAAAAAAKAAAA |
| 40 | AEKYAAAAAAKAAAA |
| 41 | AKEYAAAAAAKAAAA |
| 42 | AKKYAAAAAAKAAAA |
| 43 | AEAYAAAAAAKAAAA |
| 44 | KEAYAAAAAAKAAAA |
| 45 | AEEYAAAAAAKAAAA |
| 46 | AAEYAAAAAAKAAAA |
| 47 | EKAYAAAAAAKAAAA |
| 48 | AAKYEAAAAAKAAAA |
| 49 | AAKYAEAAAAKAAAA |
| 50 | EAAYAAAAAAKAAAA |
| 51 | EKKYAAAAAAKAAAA |
| 52 | EAKYAAAAAAKAAAA |
| 53 | AEKYAAAAAAAAAAA |
| 54 | AKEYAAAAAAAAAAA |
| 55 | AKKYEAAAAAAAAAA |
| 56 | AKKYAEAAAAAAAAA |
| 57 | AEAYKAAAAAAAAAA |
| 58 | KEAYAAAAAAAAAAA |
| 59 | AEEYKAAAAAAAAAA |
| 60 | AAEYKAAAAAAAAAA |
| 61 | EKAYAAAAAAAAAAA |
| 62 | AAKYEAAAAAAAAAA |
| 63 | AAKYAEAAAAAAAAA |
| 64 | EKKYAAAAAAAAAAA |
| 65 | EAKYAAAAAAAAAAA |
| 66 | AEYAKAAAAAAAAAA |
| 67 | AEKAYAAAAAAAAAA |
| 68 | EKYAAAAAAAAAAAA |
| 69 | AYKAEAAAAAAAAAA |
| 70 | AKYAEAAAAAAAAAA |

### Pharmaceutical Compositions/Administration

Pharmaceutical formulations of the present disclosure may be prepared by mixing an antibody (for example, an antibody that binds tau and/or an antibody that binds beta amyloid) and/or antigen-binding fragments thereof, and/or an antibody fragment, and/or a myelin sheath protein, and/or a synthetic polypeptide comprising a structure similar to said protein, having the desired degree of purity with one or more optional pharmaceutically acceptable carriers (Remington's Pharmaceutical Sciences 16th edition, Osol, A. Ed. (1980)), in the form of lyophilized formulations or aqueous solutions. The pharmaceutically acceptable carriers used according to the methods of the present disclosure are conventional. Remington: The Science and Practice of Pharmacy, The University of the Sciences in Philadelphia, Editor, Lippincott, Williams, & Wilkins, Philadelphia, Pa., 21st Edition (2005), describes compositions and formulations suitable for pharmaceutical delivery.

Methods of administration and dosages will depend on the agent/carrier used and include, but are not limited to, parenteral, e.g., intravenous, intraperitoneal, intramuscular, subcutaneous, mucosal, e.g., oral, intranasal, buccal, vaginal, rectal, or intraocular, intrathecal, topical and intradermal routes, with or without adjuvant. Administration can be systemic or local. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX(R), Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

In some embodiments, the pharmaceutical formulation may be formulated in an arginine buffer. In some embodiments, the arginine buffer may be an arginine succinate buffer. In some embodiments, the concentration of the arginine succinate buffer may be 50 mM or greater. In some embodiments, the concentration of the arginine succinate buffer may be 100 mM or greater. In some embodiments, the concentration of the arginine succinate buffer may be 150 mM or greater, or 200 mM or greater.

In some embodiments, the pharmaceutical formulation may further comprise a surfactant. In some embodiments, the surfactant is a polysorbate. In some embodiments, the polysorbate is polysorbate 20. In some embodiments, the concentration of polysorbate 20 in the formulation is 0.1% or less. In some embodiments, the concentration of polysorbate 20 in the formulation is 0.05% or less. In some embodiments, the pH of the buffer formulation is between 4.5 and 7.0. In some embodiments, the pH of the buffer formulation is between 5.0 and 6.5. In some embodiments, the pH of the buffer formulation is between 5.0 and 6.0. In some embodiments, the pH of the buffer formulation is 5.5.

Exemplary lyophilized antibody formulations are described in, for example, U.S. Pat. No. 6,267,958. In some embodiments, aqueous antibody formulations may include those described in U.S. Pat. No. 6,171,586 and WO2006/044908, the latter formulations including a histidine-acetate buffer.

In some embodiments, the formulation herein may also contain more than one active ingredient as necessary for the particular indication being treated (e.g., those with complementary activities that do not adversely affect each other). For example, it may be desirable to further provide one or more compounds to prevent or treat symptoms of a neurological disease, e.g., Alzheimer's Disease. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano- particles and nanocapsules) or in macroemulsions. Such techniques are disclosed in Remington's Pharmaceutical Sciences 16th edition, Osol, A. Ed. (1980). In some embodiments, sustained-release preparations may be prepared. Suitable examples of sustained-release preparations include semipermeable matrices of solid hydrophobic polymers containing the antibody, which matrices are in the form of shaped articles, e.g. films, or microcapsules.

In some embodiments, the present disclosure provides for pharmaceutical compositions comprising at least one antibody and/or at least one myelin sheath protein and/or a synthetic polypeptide comprising a structure similar to said protein and a pharmaceutically acceptable carrier. For therapeutic use, the antibodies of the present disclosure may be prepared as pharmaceutical compositions containing an effective amount of the antibody as an active ingredient in a pharmaceutically acceptable carrier. “Carrier” refers to a diluent, adjuvant, excipient, or vehicle with which the antibody and/or at least one myelin sheath protein or a synthetic polypeptide comprising a structure similar to said protein is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the antibody of the present disclosure in such pharmaceutical formulations may vary, from less than about 0.5%, usually to at least about 1% to as much as 15 or 20% by weight and may be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 21st Edition, Troy, D. B. ed., Lipincott Williams and Wilkins, Philadelphia, Pa. 2006, Part 5, Pharmaceutical Manufacturing pp 691-1092, See especially pp. 958-989. An exemplary pharmaceutical composition comprises 40 mg/mL antibody, 10 mM histidine, 8.5% (w/v) sucrose and 0.04% (w/v) Polysorbate 80 at pH 5.8.

Administration frequencies for a pharmaceutical composition comprising an antibody and/or a myelin sheath protein and/or a synthetic polypeptide comprising a structure similar to said protein of the present disclosure may vary based on the method being practiced, the physical characteristics of the subject, the severity of the disease, and the formulation and the means used to administer the composition. Non-limiting exemplary administration frequencies include 6, 5, 4, 3, 2 or once daily, every other day, every third day, every fourth day, every fifth day, every sixth day, once weekly, every eight days, every nine days, every ten days, bi-weekly, monthly, bi-monthly, every three months, every four months, every five months, and every six months. In some embodiments, the pharmaceutical composition is administered once daily. In some embodiments, the pharmaceutical composition is administered continuously.

In some embodiments wherein a patient's status does improve, the dose of composition being administered may be temporarily reduced or temporarily suspended for a certain length of time (i.e. a “drug holiday”). In some embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. In some embodiments, the dose reduction during a drug holiday can be a reduction of 10%-100%, including 10%, 15%, 20%, 25%, 30%, 35%, 40%, 45%, 50%, 55%, 60%, 65%, 70%, 75%, 80%, 85%, 90%, 95%, and 100%. In some embodiments, the suspension may be in regards to an antibody (e.g., antibody that binds to tau and/or beta amyloid. In some embodiments, the suspension may be in regards to a myelin sheath protein and/or a synthetic polypeptide comprising a structure similar to said protein.

In some embodiments, the dose of the composition being administered is temporarily reduced or temporarily suspended for a certain length of time. In some embodiments, the length of time is between 2 days and 1 year, including but not limited to, 2 days, 3 days, 4 days, 5 days, 6 days, 7 days, 10 days, 12 days, 15 days, 20 days, 28 days, or more than 28 days. In some embodiments, the dose reduction can be a reduction of 10%-100%, including 10%, 15%, 20%, 25%, 30%, 35%, 40%, 45%, 50%, 55%, 60%, 65%, 70%, 75%, 80%, 85%, 90%, 95%, and 100% reduction. After a suitable length of time, the normal dosing schedule is optionally reinstated.

The mode of administration for therapeutic use of the antibodies of the present disclosure and/or a myelin sheath protein and/or a synthetic polypeptide comprising a structure similar to said protein may be any suitable route that delivers the antibody and/or a myelin sheath protein and/or a synthetic polypeptide comprising a structure similar to said protein to the host. In some embodiments, the antibody and/or the myelin sheath protein and/or the synthetic polypeptide comprising a structure similar to said protein can be administered by parenteral administration, intradermal, intramuscular, intraperitoneal, intravenous or subcutaneous, pulmonary, intrapulmonary, transmucosal (oral, intranasal, intravaginal, rectal), local administration, and/or intralesional administration. In some embodiments, the formulation is in a tablet form, capsule, solution, powder, gel, particle. In some embodiments, the formulation containing the antibody and/or the myelin sheath protein and/or the synthetic polypeptide comprising a structure similar to said protein is contained in a syringe, an implanted device, osmotic pump, cartridge, micropump, or other means appreciated by the skilled artisan, as well known in the art. Site specific administration may be achieved by for example intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery. In some embodiments, the administration of the composition is brief. In some embodiments, the administration of the composition is chronic. In some embodiments, the antibody and/or the myelin sheath protein and/or the synthetic polypeptide comprising a structure similar to said protein is injected subcutaneously, or intravenously. In some embodiments, the antibody and/or the myelin sheath protein and/or the synthetic polypeptide comprising a structure similar to said protein is administered using a syringe (e.g., prefilled or not) or an autoinjector. In some embodiments, the antibody and/or the myelin sheath protein and/or the synthetic polypeptide comprising a structure similar to said protein is inhaled.

The antibodies and/or the myelin sheath protein and/or the synthetic polypeptide comprising a structure similar to said protein of the present disclosure may be administered to a subject by any suitable route, for example parentally by intravenous (i.v.) infusion or bolus injection, intramuscularly or subcutaneously or intraperitoneally. i.v. infusion may be given over for example 15, 30, 60, 90, 120, 180, or 240 minutes, or from 1, 2, 3, 4, 5, 6, 7, 8, 9, 10, 11 or 12 hours.

The dose given to a subject is sufficient to alleviate or at least partially arrest the disease being treated (“therapeutically effective amount”) and may be sometimes 0.005 mg/kg to about 100 mg/kg, e.g. about 0.05 mg/kg to about 30 mg/kg or about 5 mg/kg to about 25 mg/kg, or about 4 mg/kg, about 8 mg/kg, about 16 mg/kg or about 24 mg/kg, or for example about 0.1, 0.2, 0.3, 0.4, 0.5, 0.6, 0.7, 0.8, 0.9, 1, 2, 3, 4, 5, 6, 7, 8, 9 or 10 mg/kg, but may be even higher, for example about 15, 16, 17, 18, 19, 20, 21, 22, 23, 24, 25, 30, 40, 50, 60, 70, 80, 90 or 100 mg/kg. In some embodiments, the dose of the antibodies and/or the myelin sheath protein and/or the synthetic polypeptide comprising a structure similar to said protein of the present disclosure given to a subject may be from about 0.1 mg/kg to about 10 mg/kg via intravenous administration, or via subcutaneous administration.

A fixed unit dose of the antibodies and/or the myelin sheath protein and/or the synthetic polypeptide comprising a structure similar to said protein of the present disclosure may also be given, for example, 50, 100, 200, 500 or 1000 mg, or the dose may be based on the patient's surface area, e.g., 500, 400, 300, 250, 200, or 100 mg/m2. Usually between 1 and 8 doses, (e.g., 1, 2, 3, 4, 5, 6, 7 or 8) may be administered to treat the patient, but 9, 10, 11, 12, 13, 14, 15, 16, 17, 18, 19, 20 or more doses may be given.

The administration of the antibodies and/or the myelin sheath protein and/or the synthetic polypeptide comprising a structure similar to said protein of the present disclosure may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose. For example, the antibodies and/or the myelin sheath protein and/or the synthetic polypeptide comprising a structure similar to said protein of the present disclosure may be administered at 8 mg/kg or at 16 mg/kg at weekly interval for 8 weeks, followed by administration at 8 mg/kg or at 16 mg/kg every two weeks for an additional 16 weeks, followed by administration at 8 mg/kg or at 16 mg/kg every four weeks by intravenous infusion. In some embodiments, the antibodies and/or the myelin sheath protein and/or the synthetic polypeptide comprising a structure similar to said protein of the present disclosure may be administered at between 0.1 mg/kg to about 10 mg/kg at weekly interval for 17 weeks. For example, the antibodies and/or the myelin sheath protein and/or the synthetic polypeptide comprising a structure similar to said protein of the present disclosure may be provided as a daily dosage in an amount of about 0.1-100 mg/kg, such as about 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or about 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, and/or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

In some embodiments, an antibody and/or a myelin sheath protein and/or a synthetic polypeptide comprising a structure similar to said protein of the present disclosure is administered at a dose of about 15 mg/kg, about 30 mg/kg, about 40 mg/kg, about 45 mg/kg, about 50 mg/kg, about 60 mg/kg or a flat dose, e.g., about 300 mg, about 500 mg, about 700 mg, about 800 mg, or higher. In some embodiments, the dose is administered by intravenous injection every 2 weeks or every 4 weeks for a period of time. In some embodiments, the dose is administered by subcutaneous injection every 2 weeks or every 4 weeks for a period of time. In some embodiments, the period of time is 6 months, one year, eighteen months, two years, five years, ten years, 15 years, 20 years, or the lifetime of the patient.

In some embodiments, the appropriate dosage of an antibody and/or a myelin sheath protein and/or a synthetic polypeptide comprising a structure similar to said protein of the present disclosure (when used alone or in combination with one or more other additional therapeutic agents) may depend on the specific type of disease to be treated, the type of antibody, the myelin sheath protein, the synthetic polypeptide comprising a structure similar to said protein, the severity and course of the disease, previous therapy, the patient's clinical history and response to the antibody, and the discretion of the attending physician. The antibody and/or the myelin sheath protein and/or the synthetic polypeptide comprising a structure similar to said protein is suitably administered to the patient at one time or over a series of treatments. Various dosing schedules including, but not limited to, single or multiple administrations over various time-points, bolus administration, and/or pulse infusion.

Depending on the type and severity of the disease, about 0.3 mg/kg to 100 mg/kg (e.g. from about 15 mg/kg to about 100 mg/kg, or any dosage within that range) of antibody and/or the myelin sheath protein and/or the synthetic polypeptide comprising a structure similar to said protein can be an initial candidate dosage for administration to the patient, whether, for example, by one or more separate administrations, or by continuous infusion. One typical daily dosage might range from about 15 mg/kg to about 100 mg/kg or more. The dosage can be administered in a single dose or a divided dose (e.g., two doses of 15 mg/kg for a total dose of 30 mg/kg). For repeated administrations over several weeks or longer, depending on the condition, the treatment would generally be sustained until a desired suppression of disease symptoms occurs. One exemplary dosage of the antibody and/or the myelin sheath protein and/or the synthetic polypeptide comprising a structure similar to said protein would be in the range from about 10 mg/kg to about 50 mg/kg. Thus, one or more doses of about 0.5 mg/kg, 1 mg/kg, 1.5 mg/kg, 2.0 mg/kg, 3 mg/kg, 4.0 mg/kg, 5 mg/kg, 10 mg/kg, 15 mg/kg, 20 mg/kg, 25 mg/kg, 30 mg/kg, 35 mg/kg, 40 mg/kg, 50 mg/kg, 60 mg/kg, 70 mg/kg, 80 mg/kg, 90 mg/kg, or 100 mg/kg (or any combination thereof) may be administered to the patient. In some embodiments, the total dose administered is in the range of 50 mg to 2500 mg. An exemplary dose of about 50 mg, about 100 mg, about 200 mg, about 300 mg, about 400 mg, about 500 mg, about 600 mg, about 700 mg, about 720 mg, about 1000 mg, about 1050 mg, about 1100 mg, about 1200 mg, about 1300 mg, about 1400 mg, about 1500 mg, about 1600 mg, about 1700 mg, about 1800 mg, about 1900 mg, about 2000 mg, about 2050 mg, about 2100 mg, about 2200 mg, about 2300 mg, about 2400 mg, or about 2500 mg (or any combination thereof) may be administered to the patient. Such doses may be administered intermittently, e.g. every week, every two weeks, every three weeks, every four weeks, every month, every two months, every three months, or every six months. In some embodiments, the patient receives from one to thirty five doses (e.g. about eighteen doses of the antibody). However, other dosage regimens may be used. The progress of this therapy can be monitored by conventional techniques and assays.

The antibody and/or the myelin sheath protein and/or the synthetic polypeptide comprising a structure similar to said protein may be administered to a patient at one time or over a series of treatments. Depending on the type and severity of the disease, about 1 μg/kg to about 15 mg/kg (e.g. 0.1 mg/kg-10 mg/kg) of antibody and/or the myelin sheath protein and/or the synthetic polypeptide comprising a structure similar to said protein can be an initial candidate dosage for administration to the patient, whether, for example, by one or more separate administrations, or by continuous infusion. In some embodiments, the daily dosage might range from about 1 μg/kg to about 100 mg/kg or more. For repeated administrations over several days or longer, depending on the condition, the treatment can generally be sustained until a desired suppression of disease symptoms occurs. One exemplary dosage of the antibody and/or the myelin sheath protein and/or the synthetic polypeptide comprising a structure similar to said protein would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

The antibodies and/or the myelin sheath protein and/or the synthetic polypeptide comprising a structure similar to said protein of the present disclosure may also be administered prophylactically in order to reduce the risk of developing an inflammatory disease, and/or a disease characterized in part by beta amyloid expression, and/or a neurological disease, and/or neurological damage, and/or an amyloid disease, and/or a tauopathy, and/or a brain AO disorder, and/or a disease that may lead of cognitive impairment.

In some embodiments, the antibodies and/or the myelin sheath protein and/or the synthetic polypeptide comprising a structure similar to said protein of the present disclosure may be lyophilized for storage and reconstituted in a suitable carrier prior to use. This technique has been shown to be effective with conventional protein preparations and well known lyophilization and reconstitution techniques can be employed.

In some embodiments, the antibodies and/or the myelin sheath protein and/or the synthetic polypeptide comprising a structure similar to said protein of the present disclosure may be supplied as a sterile, frozen liquid in a glass vial with stopper and aluminum seal with flip-off cap. Each vial may contain 3.3 mL of a 50 mg/mL solution of the antibody (including a 10% overfill) in a formulation of 10 mM histidine, 8.5% (w/v) sucrose, and 0.04% (w/v) Polysorbate 80 at pH 5.8. Vials may contain no preservatives and thus may be for single use. Vials may be stored frozen and protected from light. To prepare the antibody for IV administration, the antibody formulation may be filtered with a 0.22 micron filter before being diluted in sterile diluent. Diluted antibody at volumes up to approximately 100 mL may be administered by IV infusion over a period of at least 30 minutes using an in-line 0.22 micron filter. Alternatively, the antibody may be administered as 1 or 2 subcutaneous injections of 50 mg/mL antibody in about 3.3 mL. The subcutaneous injection site may be within the abdominal area.

### Articles of Manufacture

In some embodiments, an article of manufacture containing materials useful for the treatment, prevention and/or diagnosis of the disorders described in the present disclosure is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). In some embodiments, at least one active agent in the composition is an antibody of the present disclosure. In some embodiments, at least one active agent in the composition is a myelin sheath protein and/or a synthetic polypeptide comprising a structure similar to said protein, and/or a fragment thereof. The label or package insert indicates that the composition is used for treating the condition of choice. Moreover, the article of manufacture may comprise (a) a first container with a composition contained therein, wherein the composition comprises at least one antibody of the present disclosure (if two or more antibodies are provided, the antibodies may be provided in separate containers); and (b) a second container with a composition contained therein, wherein the composition comprises at least one myelin sheath protein and/or a synthetic polypeptide comprising a structure similar to said protein. In some embodiments, the article of manufacture may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Monitoring/Assessing Response to Therapeutic Treatment

As used in methods of the present disclosure, the antibody, and/or antigen-binding fragment thereof, and/or antibody fragment, and/or the myelin sheath protein and/or a synthetic polypeptide comprising a structure similar to said protein, and/or a fragment thereof, provides therapeutic effect or benefit to the patient. In some embodiments, the antibody, and/or antigen-binding fragment thereof, and/or antibody fragment binds to beta amyloid (anti-Abeta antibody), and/or amyloid-precursor-protein, and/or amyloid beta-derived diffusible ligand, and/or a fragment thereof, and/or tau, and/or a fragment thereof. In some embodiments, the antibody, and/or antigen-binding fragment thereof, and/or antibody fragment comprises an antibody, and/or antigen-binding fragment thereof, and/or antibody fragment that binds to beta amyloid, and/or amyloid-precursor-protein, and/or amyloid beta-derived diffusible ligand, and/or a fragment thereof, and an antibody, and/or antigen-binding fragment thereof, and/or antibody fragment that binds to tau and/or a fragment thereof. In some embodiments, the antibody, and/or antigen-binding fragment thereof, and/or antibody fragment is bispecific (e.g., binds tau and amyloid beta). In some embodiments, the therapeutic benefit is a delay in, or inihibition of, progression of Alzheimer's disease (AD) or a reduction in clinical, functional, or cognitive decline. In some embodiments, therapeutic effect or benefit is reflected in a “patient response” or “response” (and grammatical variations thereof). In some embodiments, the combination of the active agents (e.g., myelin sheath protein and/or a synthetic polypeptide comprising a structure similar to said protein, and/or a fragment thereof in combination with at least one antibody) provides a synergistic effect. In some embodiments, the effect is unexpected and surprising.

In some embodiments, the antibody, and/or antigen-binding fragment thereof, and/or antibody fragment, and/or the myelin sheath protein and/or the synthetic polypeptide comprising a structure similar to said protein, and/or a fragment thereof is used to treat mild to moderate AD in a patient. The patient can be ApoE4 positive or ApoE4 negative. In some embodiments, the antibody, and/or antigen-binding fragment thereof, or antibody fragment, and/or the myelin sheath protein and/or the synthetic polypeptide comprising a structure similar to said protein, and/or a fragment thereof of the present disclosure is used to treat mild AD. In some embodiments the antibody, and/or antigen-binding fragment thereof, and/or antibody fragment, and/or the myelin sheath protein and/or the synthetic polypeptide comprising a structure similar to said protein, and/or a fragment thereof, of the present disclosure is used to treat an ApoE4 positive patient suffering from mild to moderate AD or mild AD. In some embodiments, the antibody of the present disclosure is used to treat mild AD. In some embodiments the antibody, and/or antigen-binding fragment thereof, and/or antibody fragment, and/or the myelin sheath protein and/or the synthetic polypeptide comprising a structure similar to said protein, or a fragment thereof, of the present disclosure is used to treat a patient suffering from mild AD.

In some embodiments, the patient is ‘amyloid positive,’ e.g., a patient having brain amyloid deposits that are typical of a patient diagnosed with AD or a patient having a positive florbetapir PET scan. In some embodiments, the composition of the present disclosure reduces the accumulation of brain amyloid deposits or neuritic plaques (e.g., reduce an increase in brain amyloid burden or load).

In some embodiments, the composition of the present disclosure is used to treat mild to moderate AD without increasing the incidence of ARIA-E or ARIA-H. In some embodiments, the patients are suffering from mild AD. In some embodiments, the patients are ApoE4 positive. In some embodiments, the patients are ApoE4 positive and suffering from mild AD. The term “Amyloid-Related Imaging Abnormality-Edema” or “ARIA-E” encompasses cerebral vasogenic edema and sulcal effusion. The term “Amyloid-Related Imaging Abnormality-Hemorrhage” or “ARIA-H” encompasses microhemorrhage and superficial siderosis of the central nervous system.

Patient response can be assessed using any endpoint indicating a benefit to the patient, including, without limitation, (1) inhibition, to some extent, of disease progression, including slowing down and complete arrest; (2) reduction in amount of plaque or reduction in brain amyloid accumulation; (3) improvement in one or more assessment metrics, including but not limited to Alzheimer's Disease Assessment Scale-Cognition (ADAS-Cog), Instrumental Activities of Daily Living (iADL), and Clinical Dementia Rating Scale Sum of Boxes (CDR-SOB) scales; (4) improvement in daily functioning of the patient; (5) increase in concentration of one or more biomarkers, e.g., Abeta, in cerebrospinal fluid; and (6) decrease in one or more biomarkers indicative of the presence of AD. An assessment of patient response may also include an assessment of any adverse events that may occur that may be correlated with the treatment.

In some embodiments, the cognitive ability and daily functioning of the patient is assessed prior to, during, and/or after a course of therapy with an antibody of the present disclosure. A number of cognitive and functional assessment tools have been developed for use in assessing, diagnosing, and scoring mental function, cognition, and neurological deficit.

These tools include, but are not limited to, the ADAS-Cog, including the 12 item ADAS-Cog (ADAS-Cog12), the 13-item ADAS-Cog (ADAS-Cog13), the 14-item ADAS-Cog (ADAS- Cog14); the CDR-SOB, including CDR Judgment and Problem solving and CDR Memory components; the Instrumental Activities of Daily Living (iADL); and the Mini-Mental State Examination (MMSE).

“ADAS-Cog” refers to the Alzheimer's Disease Assessment Scale Cognitive Subscale, a multi-part cognitive assessment. See Rosen et al., 1984, Amer. J. Psych. 141 : 1356-1364; Mohs et al, 1997, Alzheimer's Disease Assoc. Disorders 11(2):S13-S21. The higher the numerical score on the ADAS-Cog, the greater the tested patient's deficit or impairment relative to another individual with a lower score. The ADAS-Cog may be used as one measure for assessing whether a treatment for AD is therapeutically effective. An increase in ADAS-Cog score is indicative of worsening in the patient's condition, whereas a decrease in ADAS-Cog score denotes improvement in the patient's condition. As used herein, a ‘decline in ADAS-Cog performance’ or an ‘increase in ADAS-Cog score’ indicates a worsening in the patient's condition and may reflect progression of AD. The ADAS-Cog is an examiner-administered battery that assesses multiple cognitive domains, including memory, comprehension, praxis, orientation, and spontaneous speech (Rosen et al. 1984, Am J Psychiatr 141 : 1356-64; Mohs et al. 1997, Alzheimer Dis Assoc Disord 11(S2):S13-S21). The ADAS-Cog is a standard primary endpoint in AD treatment trials (Mani 2004, Stat Med 23:305-14). The ADAS-Cog12 is the 70-point version of the ADAS-Cog plus a 10-point Delayed Word Recall item assessing recall of a learned word list. Other ADAS-Cog scales include the ADAS-Cog13 and ADAS-Cog14.

In some embodiments, the methods of treatment provided herein provide a reduction in cognitive decline as measured by an ADAS-Cog score that is at least about 30%, at least about 35%, at least about 40%, or at least about 45% lower relative to placebo.

“CDR-SOB” refers to the Clinical Dementia Rating Scale/Sum of Boxes. See Hughes et al, 1982. CDR-assesses 6 components: memory, orientation, judgment/problem solving, community affairs, home and hobbies, and personal care. The test is administered to both the patient and the caregiver and each component (or each “box”), is scored on a scale of 0 to 3. A complete CDR-SOB score is based on the sum of the scores across all 6 boxes. Subscores can be obtained for each of the boxes or components individually as well, e.g., CDR/Memory or CDR/Judgment and Problem solving. As used herein, a “decline in CDR-SOB performance” or an “increase in CDR-SOB score” indicates a worsening in the patient's condition and may reflect progression of AD. In some embodiments, the methods of treatment provided herein provide a reduction in decline in CDR-SOB performance of at least about 30%, at least about 35%, or at least about 40% relative to placebo.

“iADL” refers to the Instrumental Activities of Daily Living scale. See Lawton, M. P., and Brody, E. M., 1969, Gerontologist 9: 179-186. This scale measures the ability to perform typical daily activities such as housekeeping, laundry, operating a telephone, shopping, preparing meals, etc. The lower the score, the more impaired the individual is in conducting activities of daily living. In some embodiments, the methods of treatment provided herein provide a reduction in decline of at least about 10%>, at least about 15%, or at least about 20% on the iADL scale relative to placebo.

Brain amyloid load or burden can be determined using neurological imaging techniques and tools, for example using PET (positron emission tomography) scanning Serial PET scans of a patient taken over time, e.g., before and after administration of a treatment (or at one or more intervals throughout the course of a treatment regimen), can permit detection of increased, decreased, or unchanged amyloid burden in the brain. This technique can further be used to determine whether amyloid accumulation is increasing or decreasing. In some embodiments, detection of amyloid deposits in the brain is performed using fiorbetapir F18 . In some embodiments, a fiorbetapir PET scan is considered positive if, based on a centralized visual read of the scan, it establishes the presence of moderate-to-frequent neuritic plaques.

### Combination Therapy

The therapeutic agents of the methods and compositions of the present disclosure may be administered simultaneously or sequentially and, when administration is sequential, any therapeutic agent (e.g., an anti-tau antibody, an antibody that binds to amyloid beta, a myelin sheath protein, or any other therapeutic agent) may be administered first, second, third, and so forth. When administration is simultaneous, the combination may be administered either in the same or different pharmaceutical composition.

An anti-tau antibody can be administered to an individual in need thereof alone (e.g., as monotherapy); or in combination therapy with one or more additional therapeutic agents. An anti-beta amyloid antibody, and/or an antibody that binds amyloid-precursor-protein, and/or an antibody that binds amyloid beta-derived diffusible ligands can be administered to an individual in need thereof alone (e.g., as monotherapy); or in combination therapy with one or more additional therapeutic agents. A myelin sheath protein and/or a synthetic polypeptide comprising a structure similar to said protein can be administered to an individual in need thereof alone (e.g., as monotherapy); or in combination therapy with one or more additional therapeutic agents. In some embodiments, the administration of an anti-tau antibody is before the administration of one or more additional therapeutic agents. In some embodiments, the administration of an anti-tau antibody is at the same time as the administration of one or more additional therapeutic agents. In some embodiments, the administration of an anti-tau antibody is after the administration of one or more additional therapeutic agents. In some embodiments, the administration of a myelin sheath protein and/or a synthetic polypeptide comprising a structure similar to said protein is before the administration of one or more additional therapeutic agents. In some embodiments, the administration of a myelin sheath protein and/or a synthetic polypeptide comprising a structure similar to said protein is at the same time as the administration of one or more additional therapeutic agents. In some embodiments, the administration of a myelin sheath protein and/or a synthetic polypeptide comprising a structure similar to said protein is after the administration of one or more additional therapeutic agents. In some embodiments, the administration of an antibody that binds amyloid-beta, and/or an antibody that binds amyloid-precursor-protein, and/or an antibody that binds amyloid beta-derived diffusible ligands, is before the administration of one or more additional therapeutic agents. In some embodiments, the administration of an antibody that binds amyloid-beta, and/or an antibody that binds amyloid-precursor-protein, and/or an antibody that binds amyloid beta-derived diffusible ligands is at the same time as the administration of one or more additional therapeutic agents. In some embodiments, the administration of an antibody that binds amyloid-beta, and/or an antibody that binds amyloid-precursor-protein, and/or an antibody that binds amyloid beta-derived diffusible ligands is after the administration of one or more additional therapeutic agents.

In some embodiments, an anti-tau antibody can be administered in combination with a myelin sheath protein and/or a synthetic polypeptide comprising a structure similar to said protein and/or an antibody that binds amyloid-beta, and/or an antibody that binds amyloid-precursor-protein, and/or an antibody that binds amyloid beta-derived diffusible ligands and/or an additional therapeutic agent. In some embodiments, a myelin sheath protein and/or a synthetic polypeptide comprising a structure similar to said protein can be administered in combination with an anti-tau antibody and/or an antibody that binds amyloid-beta and/or an antibody that binds amyloid-precursor-protein, and/or an antibody that binds amyloid beta-derived diffusible ligands, and/or an additional therapeutic agent. In some embodiments, an antibody that binds amyloid-beta, and/or an antibody that binds amyloid-precursor-protein, and/or an antibody that binds amyloid beta-derived diffusible ligands can be administered in combination with a myelin sheath protein and/or a synthetic polypeptide comprising a structure similar to said protein and/or an anti-tau antibody and/or an additional therapeutic agent.

In some embodiments, the combination therapy provides a synergistic effect. In some embodiments, administration of a myelin sheath protein and/or a synthetic polypeptide comprising a structure similar to said protein, and/or a fragment thereof, and an antibody or an antigen-binding fragment threof or antibody fragment results in a synergistic effect. In some embodiments, the antibody or an antigen-binding fragment threof or antibody fragment is an antibody or an antigen-binding fragment threof that binds tau and/or beta amyloid, and/or an antibody that binds amyloid-precursor-protein, and/or an antibody that binds amyloid beta-derived diffusible ligands. In some embodiments, the antibody or an antigen-binding fragment threof is an antibody or an antigen-binding fragment threof or antibody fragment that binds (e.g., specifically binds) to at least one of beta amyloid, and/or amyloid-precursor-protein, and/or amyloid beta-derived diffusible ligands, and an antibody or an antigen-binding fragment thereof or antibody fragment that specifically binds tau, and/or a fragment thereof. In some embodiments, the antibody or an antigen-binding fragment threof or antibody fragment is a bispecific antibody that binds to at least one of beta amyloid, and/or amyloid-precursor-protein, and/or amyloid beta-derived diffusible ligands, and/or a fragment thereof, and binds to tau, and/or a fragment thereof.

In some embodiments, the methods of the present disclosure may further comprise an additional therapeutic agent. For the treatment of AD, other suitable additional therapeutic agents include, but are not limited to, acetylcholinesterase inhibitors, including, but not limited to, Aricept (donepezil), Exelon (rivastigmine), metrifonate, and tacrine (Cognex); an anti-Abeta antibody; non-steroidal anti-inflammatory agents, including, but not limited to, ibuprofen and indomethacin; cyclooxygenase -2 (Cox2) inhibitors such as Celebrex; and monoamine oxidase inhibitors, such as Selegilene (Eldepryl or Deprenyl). Dosages for each of the above agents are known in the art. Other suitable additional therapeutic agent in the treatment of AD can be an agent that inhibits tau aggregation, e.g., a napthoquinone derivative that inhibits tau aggregation, as described in U.S. Pat. No. 7,605,179. Another suitable additional therapeutic agent is an agent that inhibits phosphorylation of tau, e.g., a 3-substituted-4-pyrimidone derivative that inhibits tau protein kinase 1, as described in U.S. Pat. No. 7,572,793.

In some embodiments, the additional therapeutic agent may include one or more agents suitable for treatment for Alzheimer's disease such as Namzaric™, Exelon®, Aricept® (donepezil hydrochloride), Namenda® (memantine hydrochloride), or galantamine hydrobromide. In some embodiments, described methods and/or compositions and/or formulations may be administered in combination with one or more treatments for Parkinson's Disease such as ABT-126 (Abbott Laboratories), pozanicline (Abbott Laboratories), MABT-5102A (AC Immune), Affitope AD-01 (AFFiRiS GmbH), Affitope AD-02 (AFFiRiS GmbH), davunetide (Allon Therapeutics Inc), nilvadipine derivative (Archer Pharmaceuticals), Anapsos (ASAC Pharmaceutical International AIE), ASP-2535 (Astellas Pharma Inc), ASP-2905 (Astellas Pharma Inc), 1 1C-AZD-2184 (AstraZeneca pic), 1 1C-AZD-2995 (AstraZeneca pic), 18F-AZD-4694 (AstraZeneca pic), AV-965 (Avera Pharmaceuticals Inc), AVN-101 (Avineuro Pharmaceuticals Inc), immune globulin intravenous (Baxter International Inc), EVP-6124 (Bayer AG), nimodipine (Bayer AG), BMS-708163 (Bristol-Myers Squibb Co), CERE-110 (Ceregene Inc), CLL-502 (CLL Pharma), CAD-106 (Cytos Biotechnology AG), mimopezil ((Debiopharm SA), DCB-AD1 (Development Centre for Biotechnology), EGb-761 ((Dr Willmar Schwabe GmbH & Co), E-2012 (Eisai Co Ltd), ACC-001 (Elan Corp pic), bapineuzumab (Elan Corp pic), ELND-006 (Elan Pharmaceuticals Inc), atomoxetine (Eli Lilly & Co), LY-2811376 (Eli Lilly & Co), LY-451395 (Eli Lilly & Co), m266 (Eli Lilly & Co), semagacestat (Eli Lilly & Co), solanezumab (Eli Lilly & Co), AZD-103 (Ellipsis Neurotherapeutics Inc), FGLL (ENKAM Pharmaceuticals A/S), EHT-0202 (ExonHit Therapeutics SA), celecoxib (GD Searle & Co), GSK-933776A (GlaxoSmithKline pic), rosiglitazone XR (GlaxoSmithKline pic), SB-742457 (GlaxoSmithKline pic), R-1578 (Hoffmann-La Roche AG), HF-0220 (Hunter-Fleming Ltd), oxiracetam (ISF Societa Per Azioni), KD-501 (Kwang Dong Pharmaceutical Co Ltd), NGX-267 (Life Science Research Israel), huperzine A (Mayo Foundation), Dimebon (Medivation Inc), MEM-1414 (Memory Pharmaceuticals Corp), MEM-3454 (Memory Pharmaceuticals Corp), MEM-63908 (Memory Pharmaceuticals Corp), MK-0249 (Merck & Co Inc), MK-0752 (Merck & Co Inc), simvastatin (Merck & Co Inc), V-950 (Merck & Co Inc), memantine (Merz & Co GmbH), neramexane (Merz & Co GmbH), Epadel (Mochida Pharmaceutical Co Ltd), 123I-MNI-330 (Molecular Neuroimaging Lie), gantenerumab (MorphoSys AG), NICS-15 (Mount Sinai School of Medicine), huperzine A (Neuro-Hitech Inc), OXIGON (New York University), NP-12 (Noscira SA), NP-61 (Noscira SA), rivastigmine (Novartis AG), ECT-AD (NsGene A/S), arundic acid (Ono Pharmaceutical Co Ltd), PF-3084014 (Pfizer Inc), PF-3654746 (Pfizer Inc), RQ-00000009 (Pfizer Inc), PYM-50028 (Phytopharm pic), Gero-46 (PN Gerolymatos SA), PBT-2 (Prana Biotechnology Ltd), PRX-03140 (Predix Pharmaceuticals Inc), Exebryl-1 (ProteoTech Inc), PF-4360365 (Rinat Neuroscience Corp), HuCAL anti-beta amyloid monoclonal antibodies (Roche AG), EVT-302 (Roche Holding AG), nilvadipine (Roskamp Institute), galantamine (Sanochemia Pharmazeutika AG), SAR-110894 (sanofi-aventis), INM-176 (Scigenic & Scigen Harvest), mimopezil (Shanghai Institute of Materia Medica of the Chinese Academy of Sciences), NEBO-178 (Stegram Pharmaceuticals), SUVN-502 (Suven Life Sciences), TAK-065 (Takeda Pharmaceutical), ispronicline (Targacept Inc), rasagiline (Teva Pharmaceutical Industries), T-817MA (Toyama Chemical), PF-4494700 (TransTech Pharma Inc), CX-717 (University of California), 18F-FDDNP (University of California Los Angeles), GTS-21 (University of Florida), 18F-AV-133 (University of Michigan), 18F-AV-45 (University of Michigan), tetrathiomolybdate (University of Michigan), 1231-IMPY (University of Pennsylvania), 18F-AV-1/ZK (University of Pennsylvania), 11C-6-Me-BTA-1 (University of Pittsburgh), 18F-6-OH-BTA-1 (University of Pittsburgh), MCD-386 (University of Toledo), leuprolide acetate implant (Voyager Pharmaceutical Corp), aleplasinin (Wyeth), begacestat (Wyeth), GSI-136 (Wyeth), NSA-789 (Wyeth), SAM-531 (Wyeth), CTS-21166 (Zapaq), and ZSET-1446 (Zenyaku Kogyo).

In some embodiments, the one additional therapeutic agent may include one or more agents useful for the treatment of motor neuronal disorders, such as AEOL-10150 (Aeolus Pharmaceuticals Inc), riluzole (Aventis Pharma AG), ALS-08 (Avicena Group Inc), creatine (Avicena Group Inc), arimoclomol (Biorex Research and Development Co), mecobalamin (Eisai Co Ltd), talampanel (Eli Lilly & Co), R-7010 (F Hoffmann-La Roche Ltd), edaravone (Mitsubishi-Tokyo Pharmaceuticals Inc), arundic acid (Ono Pharmaceutical Co Ltd), PYM-50018 (Phytopharm pic), RPI-MN (ReceptoPharm Inc), SB-509 (Sangamo Biosciences Inc), olesoxime (Trophos SA), sodium phenylbutyrate (Ucyclyd Pharma Inc), and R-pramipexole (University of Virginia).

Antibodies and/or a myelin sheath protein of the present disclosure can be used either alone or in combination with other agents in a therapy. For instance, an antibody may be co-administered with at least one additional therapeutic agent. For example, the composition according to the present disclosure may be administered in combination with other compositions comprising an additional therapeutic agent, such as a biologically active substance or compound such as, for example, a known compound used in the medication of neurodegenerative diseases, and/or tauopathies, and/or of amyloidoses, a group of diseases and disorders associated with amyloid or amyloid-like protein such as the amyloid (3 protein involved in Alzheimer's Disease.

In some embodiments, the other additional therapeutic agent may include neuron-transmission enhancers, psychotherapeutic drugs, acetylcholine esterase inhibitors, calcium-channel blockers, biogenic amines, benzodiazepine tranquillizers, acetylcholine synthesis, storage or release enhancers, acetylcholine postsynaptic receptor agonists, monoamine oxidase-A or -B inhibitors, N-methyl-D-aspartate glutamate receptor antagonists, non-steroidal anti-inflammatory drugs, antioxidants, serotonergic receptor antagonists, or other therapeutic agents. In some embodiments, the additional therapeutic agent may comprise at least one compound selected from compounds against oxidative stress, anti-apoptotic compounds, metal chelators, inhibitors of DNA repair such as pirenzepine and metabolites, 3-amino-1-propanesulfonic acid (3APS), 1,3-propanedisulfonate (1,3PDS), secretase activators, beta- and gamma-secretase inhibitors, tau proteins, anti-tau antibodies (including, but not limited to, antibodies disclosed in WO2012049570, WO2014028777, WO2014165271, WO2014100600, WO2015200806, U.S. Pat. No. 8,980,270, and U.S. Pat. No. 8,980,271), neurotransmitter, beta-sheet breakers, antiinflammatory molecules, “atypical antipsychotics” such as, for example clozapine, ziprasidone, risperidone, aripiprazole or olanzapine or cholinesterase inhibitors (ChEIs) such as tacrine, rivastigmine, donepezil, and/or galantamine and other drugs and nutritive supplements such as, for example, vitamin B 12, cysteine, a precursor of acetylcholine, lecithin, choline, Ginkgo biloba, acyetyl-L-carnitine, idebenone, propentofylline, or a xanthine derivative, together with a binding peptide according to the present disclosure including antibodies, particularly monoclonal antibodies and active fragments thereof, and, optionally, a pharmaceutically acceptable carrier and/or a diluent and/or an excipient and instructions for the treatment of diseases.

In some embodiments, a myelin sheath protein and/or an antibody of the present disclosure may be administered in combination with a neurological drug. Such neurological drugs include, but are not limited to, an antibody or other binding molecule (including, but not limited to a small molecule, a peptide, an aptamer, or other protein binder) that specifically binds to a target selected from: beta secretase, presenilin, amyloid precursor protein or portions thereof, amyloid beta peptide or oligomers or fibrils thereof, death receptor 6 (DR6), receptor for advanced glycation endproducts (RAGE), parkin, and huntingtin; an NMDA receptor antagonist (i.e., memantine), a monoamine depletor (i.e., tetrabenazine); an ergoloid mesylate; an anticholinergic antiparkinsonism agent (i.e., procyclidine, diphenhydramine, trihexylphenidyl, benztropine, biperiden and trihexyphenidyl); a dopaminergic antiparkinsonism agent (i.e., entacapone, selegiline, pramipexole, bromocriptine, rotigotine, selegiline, ropinirole, rasagiline, apomorphine, carbidopa, levodopa, pergolide, tolcapone and amantadine); a tetrabenazine; an anti-inflammatory (including, but not limited to, a nonsteroidal anti-inflammatory drug (i.e., indomethicin and other compounds listed above); a hormone (i.e., estrogen, progesterone and leuprolide); a vitamin (i.e., folate and nicotinamide); a dimebolin; a homotaurine (i.e., 3-aminopropanesulfonic acid; 3APS); a serotonin receptor activity modulator (i.e., xaliproden); an, an interferon, and a glucocorticoid or corticosteroid. The term “corticosteroid” includes, but is not limited to, fluticasone (including fluticasone propionate (FP)), beclometasone, budesonide, ciclesonide, mometasone, flunisolide, betamethasone and triamcinolone. “Inhalable corticosteroid” means a corticosteroid that is suitable for delivery by inhalation. Exemplary inhalable corticosteroids are fluticasone, beclomethasone dipropionate, budenoside, mometasone furoate, ciclesonide, flunisolide, and triamcinolone acetonide.

In some embodiments, one or more anti-amyloid beta (anti-Abeta) antibody, and/or an antibody that binds amyloid-precursor-protein, and/or an antibody that binds amyloid beta-derived diffusible ligands may be administered with an anti-tau antibody and/or a fragment thereof. In some embodiments, one or more anti-amyloid beta (anti-Abeta) antibody, and/or an antibody that binds amyloid-precursor-protein, and/or an antibody that binds amyloid beta-derived diffusible ligands may be administered with a myelin sheath protein and/or a synthetic polypeptide comprising a structure similar to said protein and/or a fragment thereof. Non-limiting examples of an anti-Abeta antibody include, but are not limited to, crenezumab, solanezumab, bapineuzumab, aducanumab, gantenerumab, and BAN-2401 (Biogen, Eisai Co., Ltd.). In some embodiments, one or more beta-amyloid aggregation inhibitors may be administered with an anti-tau antibody and/or with a myelin sheath protein and/or a synthetic polypeptide comprising a structure similar to said protein and/or a fragment thereof. Nonlimiting exemplary beta-amyloid aggregation inhibitors include ELND-005 (also referred to as AZD-103 or scyllo-inositol), tramiprosate, and PTI-80 (Exebryl-1®; ProteoTech).

In some embodiments, combination therapy can include at least one therapeutic agent including, but not limited to, cholinesterase inhibitors such as donepezil, rivastigmine, galantamine, and tacrine; NMDA antagonists such as memantine; A-beta lowering agents such as agents capable of inhibiting one or more enzymes involved in formation of A-beta, such as beta-secretase inhibitors, gamma-secretase inhibitors and gamma-secretase modulators; agents that prevent or reduce A-beta plaque building; A-beta aggregation inhibitors; RAGE antagonists; and other agents for preventing, treating or alleviating neurodegenerative diseases and/or decline in cognitive function. Specific examples of therapeutic agents are: ELND-005, Caprospinol, NRM-8499, PBT-2, Posiphen, EHT-0202, CTS-21166, Semagacest, BMS-708163, BMS-299897, BMS-433796, ELND-006, ELN-475516, ELN-318463, ELN-475513, Begacestat, E-2012, CHF-5074, Dimebolin (Latrepiridin), and PF-4494700. Such pharmaceutical combination may optionally additionally comprise a diluent, excipient, adjuvant and/or stabilizer.

In some embodiments, one or more BACE inhibitors may be administered with an anti-tau antibody, and/or with an anti-beta amyloid antibody, and/or an antibody that binds APP, and/or an antibody that binds amyloid-beta derived diffusible ligands, and/or with a myelin sheath protein and/or a synthetic polypeptide comprising a structure similar to said protein. Non-limiting examples of a BACE inhibitors include E-2609 (Biogen, Eisai Co., Ltd.), AZD3293 (also known as LY3314814; AstraZeneca, Eli Lilly & Co.), MK-8931 (verubecestat), and JNJ-54861911 (Janssen, Shionogi Pharma). In some embodiments, a BACE enzyme inhibitor can be CTS-21166, MK-8931, AZD3293, LY3314814, BI 1181181, LY2886721, E2609, RG7129, JNJ-5486911, TAK-070, or any combination thereof. In some embodiments, the at least one additional therapeutic agent is a RAGE inhibitor. In some embodiments, the RAGE inhibitor is TTP488 (Azeliragon), TTP4000, FPS-ZM1, or any combination thereof. In some embodiments, one or more tau inhibitors may be administered with an anti-tau antibody and/or with an anti-beta amyloid antibody and/or an antibody that binds APP and/or an antibody that binds amyloid-beta derived diffusible ligands and/or with a myelin sheath protein and/or a synthetic polypeptide comprising a structure similar to said protein. Non-limiting examples of tau inhibitors include methylthioninium, LMTX (also known as leuco-methylthioninium or Trx-0237; TauRx Therapeutics Ltd.), Rember™ (methylene blue or methylthioninium chloride [MTC]; Trx-0014; TauRx Therapeutics Ltd), PBT2 (Pram Biotechnology), and PTI-51-CH3 (TauPro™; ProteoTech). In some embodiments, one or more anti-tau antibodies may be administered with an anti-beta amyloid antibody and/or an antibody that binds APP and/or an antibody that binds amyloid-beta derived diffusible ligands and/or with a myelin sheath protein and/or a synthetic polypeptide comprising a structure similar to said protein. Non-limiting examples of anti-tau antibodies include BMS-986168 (Bristol-Myers Squibb) and C2N-8E12 (AbbVie, C2N Diagnostics, LLC). In some embodiments, a general misfolding inhibitor, such as NPT088 (NeuroPhage Pharmaceuticals), may be administered with an anti-tau antibody and/or with an anti-beta amyloid antibody and/or an antibody that binds APP and/or an antibody that binds amyloid-beta derived diffusible ligands and/or with a myelin sheath protein and/or a synthetic polypeptide comprising a structure similar to said protein.

In some embodiments, compositions are provided that comprise “atypical antipsychotics” such as, for example clozapine, ziprasidone, risperidone, aripiprazole or olanzapine for the treatment of positive and negative psychotic symptoms including hallucinations, delusions, thought disorders (manifested by marked incoherence, derailment, tangentiality), and bizarre or disorganized behavior, as well as anhedonia, flattened affect, apathy, and social withdrawal, together with the chimeric antibody or the humanized antibody according to the present disclosure or active fragments thereof, and, optionally, a pharmaceutically acceptable carrier and/or a diluent and/or an excipient.

Combination therapy can encompass combined administration (where two or more therapeutic agents are included in the same or separate formulations), and separate administration, in which case, administration of an antibody and/or a myelin sheath protein and/or a synthetic polypeptide comprising a structure similar to said protein, can occur prior to, simultaneously, and/or following, administration of a second agent and/or the additional therapeutic agent or agents. In some embodiments, administration of the anti-tau antibody and administration of an additional therapeutic agent occur within about one month, or within about one, two, or three weeks, or within about one, two, three, four, five, or six days, of each other. In some embodiments, administration of the anti-beta amyloid antibody and/or an antibody that binds APP and/or an antibody that binds amyloid-beta derived diffusible ligands and administration of an additional therapeutic agent occur within about one month, or within about one, two, or three weeks, or within about one, two, three, four, five, or six days, of each other. In some embodiments, administration of a myelin sheath protein and/or a synthetic polypeptide comprising a structure similar to said protein and administration of an additional therapeutic agent occur within about one month, or within about one, two, or three weeks, or within about one, two, three, four, five, or six days, of each other.

### Abeta Plaque Quantitation

From each brain, 6 pm coronal slices may be collected, and eight sections per mouse, from four different pre-determined depths throughout the region of interest (dentate gyms or cerebral cortex) may be immunostained. Histogram-based segmentation of positively stained pixels may be performed using the Image-Pro Plus software (Media Cybernetics, Bethesda, Md., USA). The segmentation algorithm may be manually applied to each image, in the dentate gyms area or in the cortical layer V, and the percentage of the area occupied by total Abeta immunostaining may be determined. Plaque numbers can be quantified from the same 6μm coronal brain slices, and may be presented as average number of plaques per brain region. Prior to quantification, slices may be coded to mask the identity of the experimental groups, and plaque burden may be quantified by an observer blinded to the identity of the groups.

### Kits

The present disclosure further provides kits for performing and/or monitoring methods described above. Typically, such kits contain an agent that specifically binds to antibodies to Abeta, and/or an agent that specifically binds to antibodies to tau, and/or an agent that specifically binds to myelin sheath protein or fragments thereof, and/or a myelin sheath protein or a fragment thereof. The kit can also include a label. For detection of antibodies to Abeta, the label is typically in the form of labeled anti-idiotypic antibodies. For detection of antibodies, the agent can be supplied prebound to a solid phase, such as to the wells of a microtiter dish. Kits also typically contain labeling providing directions for use of the kit. The labeling may also include a chart or other correspondence regime correlating levels of measured label with levels of antibodies to Abeta, and/or antibodies to tau, and/or myelin sheath protein or fragments thereof. The term labeling refers to any written or recorded material that is attached to, or otherwise accompanies a kit at any time during its manufacture, transport, sale or use. For example, the term labeling encompasses advertising leaflets and brochures, packaging materials, instructions, audio or videocassettes, computer discs, as well as writing imprinted directly on kits.

The present disclosure also provides diagnostic kits, for example, research, detection and/or diagnostic kits (e.g., for performing in vivo imaging). Such kits typically contain an antibody for binding Abeta, and/or an antibody for binding tau, and/or a myelin sheath protein or a synthetic peptide thereof. In some embodiments, the antibody is labeled or a secondary labeling reagent is included in the kit. In some embodiments, the kit is labeled with instructions for performing the intended application, for example, for performing an in vivo imaging assay.

### EXAMPLES

The following specific examples are illustrative and non-limiting. The examples described herein reference and provide non-limiting support to the various embodiments described in the preceding sections.

### Example 1: Effect of a Myelin Sheath Protein and/or Antibodies in a Mouse Model of Alzheimer's-Like Pathology

### Animals and Injections:

Male mice (160 male mice) are split into eight groups (groups of 20). Within each group, ten mice are wild type (WT) animals and ten are tandem transgenic (Tg) for both the Swedish pedigree mutation in the amyloid precursor protein (SwAPP) and the high Alzheimer's risk polymorphism in presenilin-1 (PS-1). Both the wild type controls and the transgenic mice are of the C57BI/6 background. The mice are housed in a temperature-stabilized facility on a 12:12 light:dark cycle (lights on 07:00), with food and water available ad libitum.

Starting at approximately 8 months of age, animals are administered subcutaneous injections of a myelin sheath protein (e.g., MOG, MBP, or glatiramer acetate) (group 1), or an anti-tau antibody (group 2), or an antibody that binds amyloid beta (group 3), or a myelin sheath protein and an anti-tau antibody (group 4), or a myelin sheath protein and an antibody that binds amyloid beta (group 5), or a myelin sheath protein and an anti-tau antibody and an antibody that binds amyloid beta (group 6), or a myelin sheath protein and a bispecific antibody that binds tau and amyloid beta (group 7), or control (e.g., mouse mFc) (group 8). Five Tg animals and five WT animals of each group receives a dose of between about 0.005 mg/kg to about 100 mg/kg, or between about 1 mg/kg to about 50 mg/kg, or between about 5 mg/kg to about 25 mg/kg. The rest of the mice receive mouse Fc (mFc) at the same dose and volume. Mice are weighed weekly in order to establish dosage and evaluate animal health. The injections continue for 5 months.

### Behavioral Testing:

Morris Water Maze: During the fifth month of injections, the Morris Water Maze Test is conducted to evaluate spatial learning and memory. A pool measuring 105 cm in diameter and 35cm in depth is filled with water made opaque with non-toxic white paint. The pool is then divided conceptually into four quadrants, one of which contained an escape platform hidden 2.5 cm beneath the surface. Animals are placed in a different, pseudo-randomly selected quadrant at the start of each trial, and the latency to escape the maze onto the hidden platform is measured. There are two trial blocks per day for five days, each consisting of three one-minute trials. Between trials, animals are held with a towel for approximately 30 seconds before being placed back in the water. After each block, animals are placed in a holding cage lined with towels until dry and then returned to their home cages. The testing is conducted at the same time each day with a 3-hour interval between the first and second daily blocks. Any animal not locating the platform within one minute is assigned a latency of 60 seconds and guided to the platform by hand before being removed from the water. Normal animals are expected to demonstrate a decrease in latency to escape across trials in the water maze, indicating acquisition of the location of the platform over time.

One hour after the final block of water maze acquisition testing, animals are returned to the maze for a 30 second probe trial with the platform removed to test their retention of the platform's location. Retention is assessed via a measure of the amount of time animals spent in the quadrant that formerly housed the platform (i.e., the goal quadrant). The number of times the animals swim over the spot where the platform is located is also counted. Animals with normal retention are expected to spend more time in the goal quadrant than in other quadrants and to make more platform crosses, thus demonstrating retention of the spatial location of the escape platform.

For each block, the animals' median latency to escape the maze across the three trials is recorded and these median latencies are used for statistical analysis. In addition, animals' swim speeds are estimated by dividing latency to escape by the number of maze quadrants crossed per trial to calculate mean quadrant crossing time. The median quadrant crossing time for each trial block is used as a covariate in the statistical analysis to account for potential differences in motor speed.

### Open Field:

During the fifth month, the open field test is conducted in order to explore some of the non-cognitive behavioral symptoms of Alzheimer's-like disease and whether a myelin sheath protein, or an anti-tau antibody, or an antibody that binds amyloid beta, or combinations thereof, have an effect on those symptoms. Specifically, open field can be used to measure general locomotor and exploratory activity (Walsh, R. N. & Cummins, R. A. (1976), Psychological Bulletin, 83(3), 482-504). Animals are placed into a white, box-like apparatus measuring 48×48×24 cm. The inside floor is divided into nine grids, each measuring 16×16 cm. Animals are placed in the center grid and allowed to freely explore the apparatus for a six minute trial. The number of total grid crossings is recorded as a measure of general locomotor and exploratory behavior.

### Tissue Collection and Processing:

After 5 months of treatment, the animals are sacrificed and brains are prepared for histological analysis. All animals are overdosed with a pentobarbital-based euthanasia solution and perfusion fixed. Cold heparinized isotonic (0.9%) saline is run through the body to exsanguinate the animal and animals are then perfused with 4% paraformaldehyde first in acetate, and then borate buffer (100 ml each). Upon completion of the perfusion, brains are removed and placed in 30% buffered sucrose for 3-7 days. The fixed brains are sectioned coronally at 50 μm and stored in cryoprotectant (Watson, R. E., Wiegand, S. J., Clough, R. W., & Hoffman, G. E. (1986), Peptides, 7(1), 155-159) at -20° C. until they are stained.

### Histology:

Brain tissue from the animals is used for histological analysis. Sections are stained in a 1:12 series with Congo Red to detect the presence of amyloid plaques and with cresyl violet for Nissl bodies in separate sections for visualization of all cells. Immunostaining is conducted to visualize the microglial marker Iba-1 (Millipore rabbit polyclonal anti-lba-1, 1:500). For some animals, a double-stain is conducted for both plaques and microglia in order to evaluate parameters of microglia at various distances from plaques.

### Image Analysis:

Amyloid plaque burden is analyzed by contrast analysis using the ImageJ image analysis software program (NIH). A minimum of 2 bilateral sections of hippocampus in a 1:12 series are selected and images are captured in 10x using the PictureFrame program. In each picture, the red color of the stain is isolated and the background is faded using Adobe Photoshop to achieve contrast. Identical processing parameters are used for all sections. The entire image is then converted to black and white and imported to Image J. For each image, a set percentage of the background is removed from all images and the image is converted to binary. The percentage of area stained, number of separate plaques, and average size per plaque are recorded for each animal.

The level of overall inflammation is assessed by subjective ratings. An experienced histologist blind to the experimental conditions examines cresyl violet-stained hippocampal sections at a magnification of 40×. Each animal is given a rating of “none,” “mild,” “moderate,” or “marked,” based on the presence of inflammatory cell profiles (microglia, immune cells), with “none” signifying no inflammatory cells and “severe” being the highest amount of inflammation observed. The presence of microglial-invested deposits is also noted. For each animal showing plaque-like deposits, 5 random deposits are selected from the hippocampus and the surrounding microglia-like cells are counted. The median number of microglial profiles per plaque is recorded for each animal. Finally, double stains for congo red-positive plaques and lba-l-immunoreactive microglia are used to measure the sizes of microglia contacting plaques (contact), within 20 microns of a plaque (adjacent), or greater than 30 microns from a plaque (distant).

### Statistical Analysis:

For water maze acquisition, the median latency to escape to platform is recorded for each animal for each trial block. A three-way mixed Factorial analysis of variance (ANOVA) is conducted using treatment and genotype as the between groups independent variables and block as a repeated measure. For retention, the proportion of time spent in the goal quadrant is recorded for each animal along with the number of platform crosses. Two-way ANOVAs (genotype x treatment) are conducted with each of these as dependent variables.

To evaluate locomotor activity and exploratory behavior, the number of grid crossings in the open field test is counted for each animal. A two-way factorial ANOVA is conducted using treatment and genotype as the between groups independent variables.

Two-way ANOVAs (genotype x treatment) are conducted for each of the following measures: amyloid plaque total area, plaque number, hippocampal volume, microglial size, and average plaque size. For these measures, values for individual hemispheres are averaged to calculate a mean for each animal. Finally, regression analyses are conducted to assess the predictive relationship between memory performance and plaque pathology for each treatment group.

A non-parametric log-linear analysis for qualitative variables is conducted to analyze the overall inflammation ratings. An independent group t-test is conducted to compare number of microglia-like cells surrounding hippocampal deposits in the transgenic mice of the different groups.

Data is presented as mean across all animals within a treatment/genotype group and standard error of the mean (SEM). Statistical significance is set at an alpha level of 0.05.

The following non-limiting embodiments provide illustrative examples of the invention, but do not limit the scope of the invention.

Embodiment 1. A method of treating, or delaying the onset, or the progression of a disease characterized at least in part by beta amyloid expression, activity, or deposition in a subject, or for ameliorating at least one symptom associated with the disease, the method comprising administering a therapeutically effective amount of a first agent and a second agent to the subject, wherein the first agent is a myelin sheath protein or a synthetic polypeptide comprising a structure similar to said protein, or a homolog thereof, or a fragment thereof, and the second agent is an antibody or antigen-binding fragment thereof that specifically binds beta amyloid, or amyloid-precursor-protein, or amyloid beta-derived diffusible ligands, or tau, or a fragment thereof, or any combination thereof, wherein the first agent and the second agent when administered to the subject act synergistically to reduce, inhibit, or ameliorate the symptoms or pathogenesis of the disease.

Embodiment 2. A method of improving cognitive impairment in a subject having beta amyloid deposits in brain tissue, the method comprising administering to the subject a therapeutically effective amount of at least one myelin sheath protein or a synthetic polypeptide comprising a structure similar to said protein, or a homolog thereof, or a fragment thereof, and an antibody or antigen-binding fragment thereof that specifically binds beta amyloid, or amyloid-precursor-protein, or amyloid beta-derived diffusible ligand, or tau, or a fragment thereof, or any combination thereof, wherein the subject demonstrates an improvement in cognitive function without necessarily exhibiting a concurrent change in the beta amyloid plaque burden in the brain.

Embodiment 3. A method of treating or preventing a neurological disorder, including delaying the onset, slowing the progression or ameliorating symptoms of the disorder, comprising administering a therapeutically effective amount of at least one myelin sheath protein or a synthetic polypeptide comprising a structure similar to said protein, or a homolog thereof, or a fragment thereof, before, after, or concurrently with at least one antibody or antigen-binding fragment thereof that specifically binds beta amyloid, or amyloid-precursor-protein, or amyloid beta-derived diffusible ligand, or tau, or a fragment thereof, or any combination thereof, to a subject in need thereof.

Embodiment 4. A method of treating a subject suffering from an amyloid disease or tauopathy comprising administering to the subject a therapeutically effective amount of at least one myelin sheath protein or a synthetic polypeptide comprising a structure similar to said protein, or a homolog thereof, or a fragment thereof, and at least one antibody or antigen-binding fragment thereof that specifically binds tau, or a fragment thereof and/or at least one antibody or antigen-binding fragment thereof that specifically binds beta amyloid, or amyloid-precursor-protein, or amyloid beta-derived diffusible ligand, or a fragment thereof, or any combination thereof.

Embodiment 5. A method of treating a subject having a brain Aβ disorder or predisposition to a brain Aβ disorder, comprising administering to the subject a therapeutically effective amount of at least one myelin sheath protein or a synthetic polypeptide comprising a structure similar to said protein, or a homolog thereof, or a fragment thereof, and at least one antibody or antigen-binding fragment thereof that specifically binds tau, or a fragment thereof, and/or at least one antibody or antigen-binding fragment thereof that specifically binds beta amyloid, or amyloid-precursor-protein, or amyloid beta-derived diffusible ligand, or a fragment thereof, or any combination thereof, thereby modulating production of Aβ in a tissue of the subject.

Embodiment 6. The method of embodiment 5, wherein the Aβ disorder is Alzheimer's disease.

Embodiment 7. A method of treating or preventing the progression of Alzheimer's disease or related tauopathies in a subject, the method comprising administering to the subject a therapeutically effective amount of at least one myelin sheath protein or a synthetic polypeptide comprising a structure similar to said protein, or a homolog thereof, or a fragment thereof, in combination with at least one agent selected from an inhibitor of tau aggregation, an anti-amyloid antibody, an anti-amyloid aggregation inhibitor, an antibody that binds tau, an antibody that binds beta amyloid, an antibody that binds amyloid-precursor-protein, an antibody that binds amyloid beta-derived diffusible ligands, and an antibody that binds a marker of Alzheimer's disease.

Embodiment 8. The method of embodiment 7, wherein the antibody binds at least one marker selected from the group consisting of P-S396-tau, P-T181-tau, Aβ, Aβ1-42, Aβ1-40, amyloid precursor protein (APP), amyloid precursor protein beta (APPβ), and amyloid precursor protein alpha (APPalpha).

Embodiment 9. A method of reducing the decline in functional or cognitive capacity in a subject diagnosed with early or mild to moderate Alzheimer's disease (AD) comprising administering to the subject suffering from early or mild to moderate AD a therapeutically effective amount of at least one myelin sheath protein or a synthetic polypeptide comprising a structure similar to said protein, or a homolog thereof, or a fragment thereof, in combination with at least one agent selected from an anti-amyloid antibody, an anti-tau antibody, an antibody that binds beta amyloid, an antibody that binds amyloid-precursor-protein, an antibody that binds amyloid beta-derived diffusible ligands, and an antibody that binds a marker of Alzheimer's disease, in an amount effective to slow the decline in functional or cognitive capacity in the subject.

Embodiment 10. The method of embodiment 9, wherein decline in cognitive capacity is assessed by determining the patient's score before and after administration of the antibody using a 12-item Alzheimer's disease Assessment Scale-Cognition (ADAS-Cog12), 13-item Alzheimer's disease Assessment Scale-Cognition (ADAS-Cog13), or 14-item Alzheimer's disease Assessment Scale-Cognition (ADAS-Cog14) test, optionally wherein the reduction in cognitive decline as measured by ADAS-Cog is at least 30%, at least 35%, at least 40%, or at least 45% relative to placebo.

Embodiment 11. A method of reducing plaque formation in the brain of a subject, the method comprising administering to the subject a therapeutically effective amount of at least one myelin sheath protein or a synthetic polypeptide comprising a structure similar to said protein, or a homolog thereof, or a fragment thereof, in combination with at least one agent selected from an antibody or antigen-binding fragment thereof that specifically binds beta amyloid, or amyloid-precursor-protein, or amyloid beta-derived diffusible ligands, or tau, or a fragment thereof, or any combination thereof.

Embodiment 12. A method of lessening the severity of Alzheimer's disease in the brain of a subject comprising administering a myelin sheath protein or a synthetic polypeptide comprising a structure similar to said protein, or a homolog thereof, or a fragment thereof, and an antibody or antigen-binding fragment thereof that binds to beta amyloid, or amyloid-precursor-protein, or amyloid beta-derived diffusible ligands and/or an antibody or antigen-binding fragment thereof that binds to tau, to a subject having or at risk of developing the disease, thereby lessening the severity of the disease.

Embodiment 13. The method of embodiment 3, wherein the neurological disorder is Parkinson's Disease, Alzheimer's disease, amyotrophic lateral sclerosis, stroke, a neuromuscular disorder, schizophrenia, cerebral infarction, head trauma, glaucoma, facialis, or Huntington's Disease.

Embodiment 14. The method according to any one of the preceding embodiments, wherein the myelin sheath protein or the synthetic polypeptide comprising a structure similar to said protein, or a homolog thereof, or a fragment thereof is selected from the group consisting of myelin basic protein (MBP) or a fragment thereof, myelin oligodendrocyte glycoprotein (MOG) or a fragment thereof, MBP-85-99, MOG-35-55, glatiramer acetate, or a copolymer 1 related peptide.

Embodiment 15. The method according to any one of the preceding embodiments, wherein the myelin sheath protein or a synthetic polypeptide comprising a structure similar to said protein, or a homolog thereof, or a fragment thereof, comprises, consists essentially of, or consists of an amino acid sequence selected from the group consisting of:

(SEQ ID NO: 17)
Asp-Glu-Asn-Pro-Val-Val-His-Phe-Phe-Lys-Asn-Ile-
Val-Thr-Pro-Arg-Thr;
(SEQ ID NO: 18)
Lys-Ser-His-Gly-Arg-Thr-Gln-Asp-Glu-Asn-Pro-Val-
Val-His-Phe-Phe-Lys-Asn-Ile-Val-Thr;
(SEQ ID NO: 19)
Ala-Arg-Thr-Ala-His-Tyr-Gly-Ser-Leu-Pro-Gln-Lys-
Ser-His-Gly;
(SEQ ID NO: 20)
His-His-Pro-Ala-Arg-Thr-Ala-His-Tyr-Gly-Ser-Leu-
Pro-Gln-Lys;
(SEQ ID NO: 21)
Tyr-Gly-Ser-Leu-Pro-Gln-Lys-Ser-His-Gly-Arg-Thr-
Gln-Asp-Glu;
(SEQ ID NO: 22)
Thr-Gln-Asp-Glu-Asn-Pro-Val-Val-His-Phe-Phe-Lys-
Asn-Ile-Val-Thr-Pro-Arg;
(SEQ ID NO: 23)
Lys-Asn-Ile-Val-Thr-Pro-Arg-Thr-Pro-Pro-Pro-Ser-
Gln-Gly-Lys-Gly;
(SEQ ID NO: 24)
Asn-Pro-Val-Val-His-Phe-Phe-Lys-Asn-Ile;
(SEQ ID NO: 25)
Pro-Val-Val-His-Phe-Phe-Lys-Asn-Ile-Val;
(SEQ ID NO: 26)
Val-Val-His-Phe-Phe-Lys-Asn-Ile-Val-Thr;
(SEQ ID NO: 27)
Val-His-Phe-Phe-Lys-Asn-Ile-Val-Thr-Pro;
and
(SEQ ID NO: 28)
Glu-Ala-Tyr-Lys-Ala-Ala-Glu-Lys-Ala-Tyr-Ala-Ala-
Lys-Glu-Ala-Ala-Lys-Glu-Ala-Ala-Lys-Ala-Lys-Ala-
Glu-Lys-Lys-Ala-Ala-Tyr-Ala-Lys-Ala-Lys-Ala-Ala-
Lys-Tyr-Glu-Lys-Lys-Ala-Lys-Lys-Ala-Ala-Ala-Glu-
Tyr-Lys-Lys-Lys.

Embodiment 16. The method according to any one of the preceding embodiments, wherein the antibody or antigen-binding fragment thereof that specifically binds tau binds to monomeric tau, oligomeric tau, non-phosphorylated tau, extracellular tau, and/or phosphorylated tau.

Embodiment 17. The method according to any one of the preceding embodiments, wherein the antibody or antigen-binding fragment thereof that specifically binds tau binds an epitope within amino acids 2 to 24 of mature human tau.

Embodiment 18. The method according to any one of the preceding embodiments, wherein the subject is a human.

Embodiment 19. The method according to any one of the preceding embodiments, wherein the subject has Alzheimer's disease or a genetic predisposition for developing Alzheimer's disease.

Embodiment 20. The method of embodiment 19, wherein the Alzheimer's disease is sporadic (non-hereditary) Alzheimer's disease.

Embodiment 21. The method of embodiment 19, wherein the Alzheimer's disease is familial (hereditary) Alzheimer's disease.

Embodiment 22. The method of embodiment 19, wherein the Alzheimer's disease is clinical, pre-clinical or prodromal Alzheimer's disease.

Embodiment 23. The method according to any one of the preceding embodiments, wherein the subject has mild cognitive impairment.

Embodiment 24. The method according to any one of the preceding embodiments, wherein the subject has a genomic mutation in the APP gene.

Embodiment 25. The method according to any one of the preceding embodiments, wherein the subject has a genomic mutation in the ApoE gene.

Embodiment 26. The method according to any one of the preceding embodiments, wherein the subject has a genomic mutation in a presenilin gene.

Embodiment 27. The method according to any one of the preceding embodiments, wherein the subject has familial, sporadic, or idiopathic Alzheimer's disease and/or cerebral amyloid angiopathy.

Embodiment 28. The method according to any one of the preceding embodiments, wherein the subject has amyloid deposits.

Embodiment 29. The method according to any one of the preceding embodiments, wherein the subject's brain has amyloid-beta amyloid deposits.

Embodiment 30. The method according to any one of the preceding embodiments, wherein the antibody or antigen-binding fragment thereof that specifically binds beta amyloid is capable of binding oligomeric and/or monomeric forms of beta amyloid.

Embodiment 31. The method according to any one of the preceding embodiments, wherein the subject is at risk of developing Alzheimer's disease and further wherein the subject is selected from the group consisting of subjects with mild cognitive impairment, subjects with genotypes known to be associated with Alzheimer's disease, subjects with Trisomy 21, and subjects with surrogate markers indicating risk for Alzheimer's disease.

Embodiment 32. The method according to embodiment 31, wherein the subject with genotypes known to be associated with Alzheimer's disease comprise subjects with the ApoE4 genotype.

Embodiment 33. A method of monitoring the response of a subject being treated for neurological damage by administering neuroprotective agents, comprising the steps of:
(a) determining the amount of at least one biomarker in a first biological sample taken from the subject prior to an initial treatment with a neuroprotective agent, wherein the neuroprotective agent comprises a combination of a myelin sheath protein or a synthetic polypeptide comprising a structure similar to said protein, or a homolog thereof, or a fragment thereof, and an antibody or antigen-binding fragment thereof that specifically binds tau and/or beta amyloid;
(b) determining the amount of the biomarker in at least a second biological sample taken from the subject subsequent to the initial treatment with the neuroprotective agent; and (c) comparing the amount of the biomarker in the second biological sample with the amount of the biomarker in the first biological sample; such that a detectable reduction in the amount of the biomarker in the second biological sample compared to the amount of biomarker in the first biological sample indicates that the subject is responding positively to the treatment with the neuroprotective agent.

Embodiment 34. The method of embodiment 33, wherein the biomarker is selected from the group comprising S-100b, neuron-specific enolase (NSE), glial fibrillary acidic protein (GFAP), tau protein, haptoglobin, brain creatine kinase, isoprostane, myelin basic protein (MBP), myelin oligodendrocyte glycoprotein (MOG), beta amyloid, amyloid-precursor-protein, amyloid beta-derived diffusible ligands, thrombomodulin, or a fragment thereof, or any combination thereof.

Embodiment 35. The method of embodiment 33, wherein the neurological damage is a condition or disease, or is caused by a condition, disease or event, selected from the group consisting of cerebral ischemia, cerebral infarction, head trauma, contusion, spinal cord injury, subarachnoid hemorrhage, cerebral hemorrhage, aneurysmal hemorrhage, cardiac infarction, hypoxia, anoxia, surgery, Alzheimer's disease, Parkinson's disease, multiple sclerosis, HIV-related neurodegeneration, cerebellar degeneration, seizure, and ataxia.

Embodiment 36. The method according to any one of the preceding embodiments, wherein the administration is by injection, or by infusion.

Embodiment 37. The method according to any one of the preceding embodiments, wherein the administration is subcutaneous, intramuscular, intravenous, or intradermal.

Embodiment 38. The method according to any one of the preceding embodiments, wherein the myelin sheath protein or a synthetic polypeptide comprising a structure similar to said protein, or a homolog thereof, or a fragment thereof is administered before, after, or simultaneously with the antibody or antigen-binding fragment thereof.

Embodiment 39. A method of treating Alzheimer's disease, comprising administering to a subject in need thereof a pharmaceutical composition comprising an effective amount of an antibody or antigen-binding fragment thereof that binds to soluble beta amyloid (Abeta) in combination with a myelin sheath protein or a synthetic polypeptide comprising a structure similar to said protein, or a homolog thereof, or a fragment thereof, wherein the antibody is administered peripherally to the subject to exert its beneficial effects.

Embodiment 40. The method of embodiment 39, further comprising administering an antibody or antigen-binding fragment thereof that binds tau or a fragment thereof.

Embodiment 41. The method according to any one of the preceding embodiments, wherein the antibody comprises an immunoglobulin domain.

Embodiment 42. The method according to any one of the preceding embodiments, wherein the antibody is a monoclonal antibody or a binding fragment thereof.

Embodiment 43. The method according to any one of the preceding embodiments, wherein the antibody is a chimeric antibody or a fragment thereof.

Embodiment 44. The method according to embodiment 42, wherein the monoclonal antibody or a binding fragment thereof is a human antibody, a humanized antibody, or a mouse antibody.

Embodiment 45. The method according to embodiment 42, wherein the monoclonal antibody or a binding fragment thereof inhibits formation of amyloid deposits in the subject.

Embodiment 46. The method according to embodiment 42, wherein the monoclonal antibody or a binding fragment thereof is an IgG class antibody.

Embodiment 47. The method according to embodiment 46, wherein the IgG class antibody is selected from the group consisting of IgGl, IgG2, IgG3, IgG4, and IgGM.

Embodiment 48. The method according to any one of the preceding embodiments, wherein the antibody or antigen-binding fragment thereof that specifically binds tau is selected from the group comprising BMS-986168, C2N-8E12, AADVAC-1, AADVAC-2, ACI-35, RG7345, TRx-237-015 (LMTX), AV-1451, AV-680, Posiphen, or any combination thereof.

Embodiment 49. The method according to any one of the preceding embodiments, wherein the antibody or antigen-binding fragment thereof that specifically binds beta amyloid, or amyloid-precursor-protein, or amyloid beta-derived diffusible ligands, is selected from the group comprising LY2062430, RN-1219, R-1450, humanized m266, or any combination thereof.

Embodiment 50. The method according to any one of the preceding embodiments, wherein the antibody or antigen-binding fragment thereof that specifically binds beta amyloid, or amyloid-precursor-protein, or amyloid beta-derived diffusible ligands, binds Abeta 42.

Embodiment 51. The method according to any one of the preceding embodiments, wherein the antibody or antigen-binding fragment thereof that specifically binds beta amyloid, or amyloid-precursor-protein, or amyloid beta-derived diffusible ligands thereof, binds Abeta 40.

Embodiment 52. The method according to any one of the preceding embodiments, wherein the antibody or antigen-binding fragment thereof that specifically binds beta amyloid, or amyloid-precursor-protein, or amyloid beta-derived diffusible ligands, binds an intracellular Abeta.

Embodiment 53. The method according to any one of the preceding embodiments, wherein the antibody or antigen-binding fragment thereof is a bispecific antibody or an antigen-binding fragment having two antigen-binding regions.

Embodiment 54. The method according to embodiment 53, wherein one of the antigen-binding regions of the bispecific antibody or of the antigen-binding fragment having two antigen-binding regions binds to human tau or a fragment thereof.

Embodiment 55. The method according to embodiment 53, wherein one of the antigen-binding regions of the bispecific antibody or of the antigen-binding fragment having two antigen-binding regions binds to human amyloid protein or a fragment thereof.

Embodiment 56. The method according to embodiment 53, wherein the first antigen-binding region of the bispecific antibody or of the antigen-binding fragment having two antigen-binding regions binds to human beta amyloid, or amyloid-precursor-protein, or amyloid beta-derived diffusible ligands, or a fragment thereof, and the second antigen-binding region of the bispecific antibody or of the antigen-binding fragment having two antigen-binding regions binds to human tau or a fragment thereof.

Embodiment 57. The method according to embodiment 53, wherein the bispecific antibody or the antigen-binding fragment having two antigen-binding regions thereof comprises a first specificity towards: a) an epitope of beta-amyloid peptide that contains residues 28-35 of beta-amyloid; b) an epitope of beta-amyloid peptide that contains residues 28-34 of beta-amyloid; c) an epitope of beta-amyloid peptide that contains residues 28-33 of beta-amyloid; or d) an epitope within the region of residues 28-35 of beta-amyloid; and a second specificity towards a tau protein or a fragment thereof.

Embodiment 58. The method according to any one of the preceding embodiments, wherein the administering of myelin sheath protein or a synthetic polypeptide comprising a structure similar to said protein, or a homolog thereof, or a fragment thereof, and the antibody or the antigen-binding fragment thereof results in a synergistic effect.

Embodiment 59. The method according to any one of the preceding embodiments, wherein the antibody or the antigen-binding fragment thereof specifically binds to beta amyloid, or amyloid-precursor-protein, or amyloid beta-derived diffusible ligands, or a fragment thereof, and to tau, or a fragment thereof.

Embodiment 60. The method according to any one of the preceding embodiments, wherein the antibody or the antigen-binding fragment thereof comprises an antibody or an antigen-binding fragment thereof that specifically binds beta amyloid, or amyloid-precursor-protein, or amyloid beta-derived diffusible ligands, and an antibody or an antigen-binding fragment thereof that specifically binds tau, or a fragment thereof.

Embodiment 61. The method according to any one of the preceding embodiments, wherein the synthetic polypeptide is at least about 80%, at least about 85%, at least about 90%, at least about 95%, at least about 97%, at least about 98%, or at least about 99% identical to a myelin sheath protein or a fragment thereof.

What is claimed is:

1. A method of treating Alzheimer's disease, comprising administering to a subject in need thereof a pharmaceutical composition comprising an effective amount of an antibody or antigen-binding fragment thereof that binds to soluble beta amyloid (Abeta) in combination with a myelin sheath protein or a synthetic polypeptide comprising a structure similar to the protein, or a homolog thereof, or a fragment thereof, wherein the antibody or antigen-binding fragment thereof that binds to soluble beta amyloid (Abeta) is administered peripherally to the subject to exert its beneficial effects.

2. The method of claim 1, wherein the myelin sheath protein or the synthetic polypeptide comprising a structure similar to the protein, or a homolog thereof, or a fragment thereof, comprises, consists essentially of, or consists of an amino acid sequence selected from the group consisting of:
(SEQ ID NO: 17)
Asp-Glu-Asn-Pro-Val-Val-His-Phe-Phe-Lys-Asn-Ile-
Val-Thr-Pro-Arg-Thr;
(SEQ ID NO: 18)
Lys-Ser-His-Gly-Arg-Thr-Gln-Asp-Glu-Asn-Pro-Val-
Val-His-Phe-Phe-Lys-Asn-Ile-Val-Thr;
(SEQ ID NO: 19)
Ala-Arg-Thr-Ala-His-Tyr-Gly-Ser-Leu-Pro-Gln-Lys-
Ser-His-Gly;
(SEQ ID NO: 20)
His-His-Pro-Ala-Arg-Thr-Ala-His-Tyr-Gly-Ser-Leu-
Pro-Gln-Lys;
(SEQ ID NO: 21)
Tyr-Gly-Ser-Leu-Pro-Gln-Lys-Ser-His-Gly-Arg-Thr-
Gln-Asp-Glu;
(SEQ ID NO: 22)
Thr-Gln-Asp-Glu-Asn-Pro-Val-Val-His-Phe-Phe-Lys-
Asn-Ile-Val-Thr-Pro-Arg;
(SEQ ID NO: 23)
Lys-Asn-Ile-Val-Thr-Pro-Arg-Thr-Pro-Pro-Pro-Ser-
Gln-Gly-Lys-Gly;
(SEQ ID NO: 24)
Asn-Pro-Val-Val-His-Phe-Phe-Lys-Asn-Ile;
(SEQ ID NO: 25)
Pro-Val-Val-His-Phe-Phe-Lys-Asn-Ile-Val;
(SEQ ID NO: 26)
Val-Val-His-Phe-Phe-Lys-Asn-Ile-Val-Thr;
(SEQ ID NO: 27)
Val-His-Phe-Phe-Lys-Asn-Ile-Val-Thr-Pro;
and
(SEQ ID NO: 28)
Glu-Ala-Tyr-Lys-Ala-Ala-Glu-Lys-Ala-Tyr-Ala-Ala-
Lys-Glu-Ala-Ala-Lys-Glu-Ala-Ala-Lys-Ala-Lys-Ala-
Glu-Lys-Lys-Ala-Ala-Tyr-Ala-Lys-Ala-Lys-Ala-Ala-
Lys-Tyr-Glu-Lys-Lys-Ala-Lys-Lys-Ala-Ala-Ala-Glu-
Tyr-Lys-Lys-Lys.

3. The method of claim 1, wherein the antibody or antigen-binding fragment thereof that binds to soluble beta amyloid (Abeta) comprises an immunoglobulin domain.

4. The method of claim 1, wherein the antibody is a monoclonal antibody or a binding fragment thereof

5. The method of claim 1, wherein the antibody is a chimeric antibody or a fragment thereof.

6. The method of claim 4, wherein the monoclonal antibody or binding fragment thereof is a human antibody, a humanized antibody, or a mouse antibody.

7. The method of claim 4, wherein the monoclonal antibody or binding fragment thereof inhibits formation of amyloid deposits in the subject.

8. The method of claim 4, wherein the monoclonal antibody or binding fragment thereof is an IgG class antibody.

9. The method of claim 8, wherein the IgG class antibody is selected from the group consisting of IgG1, IgG2, IgG3, IgG4, and IgGM.

10. The method of claim 1, wherein the antibody or antigen-binding fragment thereof that binds to soluble beta amyloid (Abeta) is selected from the group comprising LY2062430, RN-1219, R-1450, humanized m266, or any combination thereof.

11. The method of claim 1, wherein the antibody or antigen-binding fragment thereof that binds to soluble beta amyloid (Abeta) binds Abeta 42.

12. The method of claim 1, wherein the antibody or antigen-binding fragment thereof that binds to soluble beta amyloid (Abeta) binds Abeta 40.

13. The method of claim 1, wherein the antibody or antigen-binding fragment thereof that binds to soluble beta amyloid (Abeta) binds an intracellular Abeta.

14. The method of claim 1, wherein the antibody or antigen-binding fragment thereof is a bispecific antibody having two antigen-binding regions or an antigen-binding fragment having two antigen-binding regions.

15. The method of claim 14, wherein one of the antigen-binding regions of the bispecific antibody or of the antigen-binding fragment having two antigen-binding regions binds to human tau or a fragment thereof.

16. The method of claim 14, wherein one of the antigen-binding regions of the bispecific antibody or of the antigen-binding fragment having two antigen-binding regions binds to a human amyloid protein or a fragment thereof.

17. The method of claim 14, wherein one of the antigen-binding region of the bispecific antibody or of the antigen-binding fragment having two antigen-binding regions binds to human beta amyloid, or amyloid-precursor-protein, or amyloid beta-derived diffusible ligands, or a fragment thereof, and the other antigen-binding region of the bispecific antibody or of the antigen-binding fragment having two antigen-binding regions binds to human tau or a fragment thereof.

18. The method of claim 14, wherein the bispecific antibody or the antigen-binding fragment having two antigen-binding regions thereof comprises a first specificity towards: a) an epitope of beta-amyloid peptide that contains residues 28-35 of beta-amyloid; b) an epitope of beta-amyloid peptide that contains residues 28-34 of beta-amyloid; c) an epitope of beta-amyloid peptide that contains residues 28-33 of beta-amyloid; or d) an epitope within the region of residues 28-35 of beta-amyloid; and a second specificity towards a tau protein or a fragment thereof.

19. The method of claim 1, wherein the administering of myelin sheath protein or a synthetic polypeptide comprising a structure similar to the protein, or a homolog thereof, or a fragment thereof, and the antibody or the antigen-binding fragment thereof that binds to soluble beta amyloid (Abeta) results in a synergistic effect.

20. The method of claim 1, wherein the synthetic polypeptide comprises an amino acid sequence at least about 80%, at least about 85%, at least about 90%, at least about 95%, at least about 97%, at least about 98%, or at least about 99% identical to a myelin sheath protein or a fragment thereof.

21. The method of claim 1, further comprising administering an antibody or antigen-binding fragment thereof that binds tau or a fragment thereof.

22. The method of claim 21, wherein the antibody or antigen-binding fragment thereof that binds tau is selected from the group comprising BMS-986168, C2N-8E12, AADVAC-1, AADVAC-2, ACI-35, RG7345, TRx-237-015 (LMTX), AV-1451, AV-680, Posiphen, or any combination thereof.

23. The method of claim 1, wherein the subject has a genomic mutation in the amyloid precursor protein (APP) gene.

24. The method of claim 1, wherein the subject has a genomic mutation in the apolipoprotein E (ApoE) gene.

25. The method of claim 1, wherein the subject has a genomic mutation in a presenilin gene.
